SEQUENCE LISTING (52 sequences)

SEQ ID NO: 1 - DNA, 20 bp, Streptococcus pyogenes
  source 1..20: mol_type = genomic DNA
ggaacctctagtggtgaagg

SEQ ID NO: 2 - DNA, 20 bp, Streptococcus pyogenes
  source 1..20: mol_type = genomic DNA
catagctgatggtacccccc

SEQ ID NO: 3 - DNA, 20 bp, Streptococcus pyogenes
  source 1..20: mol_type = genomic DNA
ggtgggggcagctgacaacc

SEQ ID NO: 4 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 24 aa, synthetic construct
IYIWAPLAGTCGVLLLSLVITLYC

SEQ ID NO: 6 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 7 - protein, 41 aa, synthetic construct
KRGRKKLLYIFKQPFMRVQTTQEEDGCSCRFPEEEEGGCEL

SEQ ID NO: 8 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - DNA, 23 bp, synthetic construct
  source 1..23: mol_type = other DNA
gccgacgcgaaatcttagcgnrg

SEQ ID NO: 10 - DNA, 20 bp, Streptococcus pyogenes
  source 1..20: mol_type = genomic DNA
tagctcacgacataatttgg

SEQ ID NO: 11 - DNA, 20 bp, synthetic construct
  source 1..20: mol_type = other DNA
gggatcactctcggcatgga

SEQ ID NO: 12 - DNA, 13 bp, synthetic construct
  source 1..13: mol_type = other DNA
ggtgggggcagct

SEQ ID NO: 13 - DNA, 24 bp, synthetic construct
  source 1..24: mol_type = other DNA
tcttctcctggttgtcagctgccc

SEQ ID NO: 14 - DNA, 23 bp, synthetic construct
  source 1..23: mol_type = other DNA
tcttctcctggtgtcagctgccc

SEQ ID NO: 15 - DNA, 23 bp, synthetic construct
  source 1..23: mol_type = other DNA
tcttctcctggttgtcagctgcc

SEQ ID NO: 16 - DNA, 24 bp, synthetic construct
  source 1..24: mol_type = other DNA
tcttctcctggtttgtcagctgcc

SEQ ID NO: 17 - DNA, 24 bp, synthetic construct
  source 1..24: mol_type = other DNA
tcttctcctggttgtcagctgccc

SEQ ID NO: 18 - DNA, 21 bp, synthetic construct
  source 1..21: mol_type = other DNA
tcttctcctggtcagctgccc

SEQ ID NO: 19 - DNA, 24 bp, synthetic construct
  source 1..24: mol_type = other DNA
tcttctcctggttgtcagctgccc

SEQ ID NO: 20 - DNA, 22 bp, synthetic construct
  source 1..22: mol_type = other DNA
tcttctcctgtgtcagctgccc

SEQ ID NO: 21 - DNA, 24 bp, synthetic construct
  source 1..24: mol_type = other DNA
tcttctcctggttgtcagctgccc

SEQ ID NO: 22 - DNA, 20 bp, synthetic construct
  source 1..20: mol_type = other DNA
tcttctcctgtcagctgccc

SEQ ID NO: 23 - DNA, 24 bp, synthetic construct
  source 1..24: mol_type = other DNA
tcttctcctggttgtcagctgccc

SEQ ID NO: 24 - DNA, 22 bp, synthetic construct
  source 1..22: mol_type = other DNA
tcttctcctggttcagctgccc

SEQ ID NO: 25 - DNA, 13 bp, synthetic construct
  source 1..13: mol_type = other DNA
tcttctcctgccc

SEQ ID NO: 26 - DNA, 18 bp, synthetic construct
  source 1..18: mol_type = other DNA
tcttcttgtcagctgccc

SEQ ID NO: 27 - DNA, 19 bp, synthetic construct
  source 1..19: mol_type = other DNA
tcttctctgtcagctgccc

SEQ ID NO: 28 - DNA, 10 bp, synthetic construct
  source 1..10: mol_type = other DNA
tcagctgccc

SEQ ID NO: 29 - DNA, 12 bp, synthetic construct
  source 1..12: mol_type = other DNA
tcttctcctggt

SEQ ID NO: 30 - DNA, 13 bp, synthetic construct
  source 1..13: mol_type = other DNA
ggaacctctagtg

SEQ ID NO: 31 - DNA, 24 bp, synthetic construct
  source 1..24: mol_type = other DNA
ctctagtggtgaaggtggaaggta

SEQ ID NO: 32 - DNA, 18 bp, synthetic construct
  source 1..18: mol_type = other DNA
ctctagtggtggaaggta

SEQ ID NO: 33 - DNA, 15 bp, synthetic construct
  source 1..15: mol_type = other DNA
ctctagtggaaggta

SEQ ID NO: 34 - DNA, 23 bp, synthetic construct
  source 1..23: mol_type = other DNA
ctctagtggtgaaggtggaaggt

SEQ ID NO: 35 - DNA, 24 bp, synthetic construct
  source 1..24: mol_type = other DNA
ctctagtggtgaaaggtggaaggt

SEQ ID NO: 36 - DNA, 23 bp, synthetic construct
  source 1..23: mol_type = other DNA
ctctagtggtgaggtggaaggta

SEQ ID NO: 37 - DNA, 16 bp, synthetic construct
  source 1..16: mol_type = other DNA
ctctaggtggaaggta

SEQ ID NO: 38 - DNA, 18 bp, synthetic construct
  source 1..18: mol_type = other DNA
ctctagtggtgaaaggta

SEQ ID NO: 39 - DNA, 14 bp, synthetic construct
  source 1..14: mol_type = other DNA
ctctagtggtggta

SEQ ID NO: 40 - DNA, 22 bp, synthetic construct
  source 1..22: mol_type = other DNA
ctctagtggtgagtggaaggta

SEQ ID NO: 41 - DNA, 18 bp, synthetic construct
  source 1..18: mol_type = other DNA
ctctagaggtggaaggta

SEQ ID NO: 42 - DNA, 11 bp, synthetic construct
  source 1..11: mol_type = other DNA
ctctagtggta

SEQ ID NO: 43 - DNA, 16 bp, synthetic construct
  source 1..16: mol_type = other DNA
ctctagtggtgaggta

SEQ ID NO: 44 - DNA, 24 bp, synthetic construct
  source 1..24: mol_type = other DNA
ctctagtggtgacaggtggaaggt

SEQ ID NO: 45 - DNA, 10 bp, synthetic construct
  source 1..10: mol_type = other DNA
ttctcctggt

SEQ ID NO: 46 - DNA, 10 bp, synthetic construct
  source 1..10: mol_type = other DNA
tgtcagctgc

SEQ ID NO: 47 - DNA, 10 bp, synthetic construct
  source 1..10: mol_type = other DNA
ctagtggtga

SEQ ID NO: 48 - DNA, 10 bp, synthetic construct
  source 1..10: mol_type = other DNA
aggtggaagg

SEQ ID NO: 49 - DNA, 16 bp, synthetic construct
  source 1..16: mol_type = other DNA
ttctcctggtggaagg

SEQ ID NO: 50 - DNA, 21 bp, synthetic construct
  source 1..21: mol_type = other DNA
gataaatctggtcttatttcc

SEQ ID NO: 51 - DNA, 23 bp, synthetic construct
  source 1..23: mol_type = other DNA
  misc_feature 1..18: note = DNA
  misc_feature 19..23: note = AA
tagctcacgacataatttdgnrg

SEQ ID NO: 52 - DNA, 23 bp, synthetic construct
  source 1..23: mol_type = other DNA
tagctcacgacataatttggtgg

(19) United States
(12) Patent Application Publication
(10) Pub. No.: US 2025/0122534 A1
(43) Pub. Date: Apr. 17, 2025

(54) COMPOSITIONS AND METHODS FOR GENE MODIFICATION

(71) Applicant: VOR BIOPHARMA INC., Cambridge, MA (US)

(72) Inventors: Tirtha Chakraborty, Cambridge, MA (US); Elizabeth Paik, Cambridge, MA (US); Michelle Lin, Cambridge, MA (US); Juliana Ferrucio, Cambridge, MA (US)

(73) Assignee: VOR BIOPHARMA INC., Cambridge, MA (US)

(21) Appl. No.: 18/293,882

(22) PCT Filed: Aug. 2, 2022
(86) PCT No.: PCT/US2022/074423

§ 371 Date: Jan. 31, 2024

Related U.S. Application Data
(60) Provisional application No. 63/346,819, filed on May 27, 2022.
(60) Provisional application No. 63/341,346, filed on May 12, 2022.
(60) Provisional application No. 63/229,484, filed on Aug. 4, 2021.
(60) Provisional application No. 63/228,548, filed on Aug. 2, 2021.

(51) Int. Cl. C12N 15/90; A61K 35/28; A61P 35/00; C12N 9/22; C12N 15/11

(52) U.S. Cl. CPC C12N 15/907; A61K 35/28; A61P 35/00; C12N 9/22; C12N 15/11; C12N 2310/20; C12N 2310/315; C12N 2310/321

(57) ABSTRACT

Provided herein are methods of making genetically engineered cells having a plurality of modifications (e.g., insertions or deletions), cells and cell populations produced by said methods, methods involving administering such genetically engineered cells to a subject, such as a subject having a hematopoietic malignancy.

### RELATED APPLICATIONS

The application claims the benefit under 35 U.S.C. 119(e) of U.S. Provisional Application No. 63/228,548 filed Aug. 2, 2021, U.S. Provisional Application No. 63/229,484 filed Aug. 4, 2021, U.S. Provisional Application No. 63/341,346 filed May 12, 2022, and U.S. Provisional Application No. 63/346,819 filed May 27, 2022, which are incorporated by reference in its entirety.

### REFERENCE TO AN ELECTRONIC SEQUENCE LISTING

The contents of the electronic sequence listing (V029170018W000-SEQ-CEW.xml; Size: 47,752 bytes; and Date of Creation: Aug. 2, 2022) is herein incorporated by reference in its entirety.

### BACKGROUND

Clustered regulatory Interspaced Short Palindromic Repeats (CRISPR)/Cas systems provide a platform for targeted gene editing in cells. Despite the versatility of the systems and associated tools for use, there are a number of potential risks associated with genetic modification using CRISPR/Cas systems, such as off-target effects, risk of translocation events, and potential malignancy. These challenges are safety concerns for use of CRISPR/Cas systems in therapeutic applications.

### SUMMARY

Aspects of the present disclosure provide methods for generating genetically engineered cells comprising multiple genetic modifications, which may be used, for example, for use therapeutic approaches. The methods provided herein aim to reduce detrimental effects of making multiple genetic modifications in the genome of a cells. In one aspect, the disclosure relates to the discovery that the sequential order of genetic modification produces a plurality of modifications in the cell and minimizes risk of generating translocation products. Without wishing to be bound by theory, it is thought that reducing the time in which multiple breaks in the genomic DNA coexist within a cell may decrease production of translocation products. It is thought that breaks in genomic DNA can be recognized and repaired by a variety of cellular DNA repair processes and that certain breaks (e.g., breaks in certain genomic loci, breaks produced by certain genetic editing processes) may be preferentially recognized by particular cellular DNA repair processes and repaired more quickly as compared to breaks recognized and repaired by other DNA repair processes. The duration of a break in the genomic DNA and rate of repair may be influenced by the cellular DNA repair process that recognizes/repairs the break. The disclosure is directed, in part, to methods comprising a first modification step introducing a first break in the genomic DNA and a second modification step introducing a second break in the genomic DNA, wherein the first break is substantially repaired or resolved (e.g., a genetic modification has been produced) prior to introduction of the second break. In some embodiments, the first break is recognized by a DNA repair process that quickly resolves/repairs the break, e.g., relative to other DNA repair processes. In some embodiments, the second break introduced is recognized by a DNA repair process that more slowly resolves/repairs the break, e.g., relative to other DNA repair processes.

Aspects of the present disclosure provide methods comprising a) contacting a plurality of cells with (i) a first gRNA comprising a first targeting domain that binds to a first target sequence and (ii) an RNA guided-nuclease that binds the first gRNA, thus forming a first ribonucleoprotein (RNP) complex under conditions suitable for the first gRNA to form and/or maintain a first RNP complex with the RNA-guided nuclease of (ii) and for the first RNP complex to bind the first target sequence; and b) contacting the plurality of cells with (iii) a second gRNA comprising a second targeting domain that binds a second target sequence and (iv) an RNA guided nuclease that binds the second gRNA to form and/or maintain a second RNP complex with the RNA guided nuclease of (iv) and for the second RNP complex to bind the second target sequence; thereby producing a population of genetically engineered cells comprising a genetic modification of the first target sequence and a genetic modification of the second target sequence; wherein steps (a) and (b) are performed sequentially and in temporal proximity, separated by a time interval, wherein the first targeting domain is not identical to the second targeting domain.

In some embodiments, the genetic modification of the first target sequence consists of an insertion or deletion at or immediately proximal to a site cut by the RNA-guided nuclease when bound to the first gRNA; and/or the genetic modification of the second target sequence consists of an insertion or deletion immediately proximal to a site cut by the RNA-guided nuclease when bound to the second gRNA. In some embodiments, the method produces a population of translocation product cells, wherein each cell of the subpopulation comprises a translocation product comprising a portion of the genome comprising the first target sequence, a portion of the genome comprising the second target sequence, or both.

In some embodiments, the method produces fewer translocation product cells as compared to a method comprising contacting a plurality of cells with the second gRNA of (iii) prior to contacting the plurality of cells with the first gRNA of (i). In some embodiments, the method produces at least 10% fewer translocation product cells as compared to a method comprising contacting the plurality of cells with the second gRNA of (iii) prior to contacting the plurality of cells with the first gRNA of (i). In some embodiments, the method produces fewer translocation product cells as compared to a method comprising contacting the plurality of cells with the first gRNA of (i) and the second gRNA of (iii) at substantially the same time. In some embodiments, the method produces at least 10% fewer translocation product cells as compared to a method comprising contacting the plurality of cells with the first gRNA of (i) and the second gRNA of (iii) at substantially the same time.

In some embodiments, binding of the first RNP complex comprising (i) and (ii) to the first target sequence results in a genetic modification generated by a Non-Homologous End Joining (NHEJ) event. In some embodiments, binding of the RNP complex comprising (i) and (ii) to the first target sequence produces a fast-resolving double strand break. In some embodiments, binding of the second RNP complex comprising (iii) and (iv) to the second target sequence results in a genetic modification generated by a microhomology-mediated end joining (MMEJ) event. In some embodiments, binding of the second RNP complex comprising (iii) and (iv) to the second target sequence produces a slow-resolving double strand break.

In some embodiments, the first target sequence is present in a first gene, a transcriptional control element operably linked thereto, or a portion of the gene and transcriptional control element. In some embodiments, the genomic modification of the first target sequence results in reduced or eliminated expression of the product encoded by the first gene, or expression of a variant of the product expressed by wild-type cells of the same cell type that do not harbor a genomic modification in the first target sequence.

In some embodiments, the first gene encodes a first lineage-specific cell-surface antigen. In some embodiments, the first lineage-specific cell-surface antigen is selected from the group consisting of CD33, CD19, CD123, CLL-1, CD30, CD5, CD6, CD7, CD38, and BCMA. In some embodiments, the second target sequence is present in a second gene, a transcriptional control element operably linked thereto, or a portion of the gene and transcriptional control element. In some embodiments, the genomic modification of the second target sequence results in reduced or eliminated expression of the product encoded by the second gene, or expression of a variant of the product expressed by wild-type cells of the same cell type that do not harbor a genomic modification in the second target sequence. In some embodiments, the second gene encodes a second lineage-specific cell-surface antigen. In some embodiments, the second lineage-specific cell-surface antigen is selected from the group consisting of CD33, CD19, CD123, CLL-1, CD30, CD5, CD6, CD7, CD38, and BCMA. In some embodiments, the first lineage-specific cell-surface antigen is CD33. In some embodiments, the second lineage-specific cell-surface antigen is CD19, CD5, or CLL-1. In some embodiments, the first lineage-specific cell-surface antigen is CD5. In some embodiments, the second lineage-specific cell-surface antigen is CD33.

In some embodiments, the first lineage-specific cell-surface antigen is CD33 and the second lineage-specific cell-surface antigen is CLL-1. In some embodiments, the first lineage-specific cell-surface antigen is CLL-1 and the second lineage-specific cell-surface antigen is CD33.

In some embodiments, the time interval between step (b) and step (c) is at least 4, 6, 8, 10, 12, 14, 16, 18, 20, 22, 24, 26, 28, 30, 32, 34, or 36 hours.

In some embodiments, the RNA-guided nuclease of (ii) and/or the RNA-guided nuclease of (iv) is a CRISPR/Cas nuclease. In some embodiments, the CRISPR/Cas nuclease is a Cas9 nuclease. In some embodiments, the CRISPR/Cas nuclease is an spCas nuclease. In some embodiments, the CRISPR/Cas nuclease is an saCas nuclease. In some embodiments, the CRISPR/Cas nuclease is a Cpf1 nuclease. In some embodiments, the RNA-guided nuclease of (ii) is a Cas9 nuclease and the RNA-guided nuclease of (iv) is a Cpf1 nuclease. In some embodiments, the RNA-guided nuclease of (ii) is a Cpf1 nuclease and the RNA-guided nuclease of (iv) is a Cas9 nuclease. In some embodiments, the RNA-guided nuclease of (ii) and the RNA-guided nuclease of (iv) are Cpf1 nucleases. In some embodiments, the RNA-guided nuclease of (ii) and the RNA-guided nuclease of (iv) are Cas9 nucleases.

In some embodiments, the contacting of (a) comprises introducing (i) and (ii) into the cell in the form of a pre-formed ribonucleoprotein (RNP) complex; and/or wherein the contacting of (b) comprises introducing (iii) and (iv) into the cell in the form of a pre-formed ribonucleoprotein (RNP) complex. In some embodiments, the pre-formed ribonucleoprotein (RNP) complex is introduced into the cell via electroporation. In some embodiments, the contacting of (a) comprises introducing (i) and/or (ii) into the cell in the form of a nucleic acid encoding the gRNA of (i) and/or the RNA-guided nuclease of (ii); and/or wherein the contacting of (b) comprises introducing (iii) and/or (iv) into the cell in the form of a nucleic acid encoding the gRNA of (i) and/or the RNA-guided nuclease of (ii).

In some embodiments, the nucleic acid encoding the first gRNA of (i) and/or the RNA-guided nuclease of (ii) is an RNA, preferably an mRNA or an mRNA analog. In some embodiments, the nucleic acid encoding the second gRNA of (iii) and/or the RNA-guided nuclease of (iv) is an RNA, preferably an mRNA or an mRNA analog. In some embodiments, the first gRNA and/or the second gRNA comprises one or more nucleotide residues that are chemically modified. In some embodiments, the first and/or the second gRNA comprises one or more nucleotide residues that comprise a 2′O-methyl moiety. In some embodiments, the first and/or the second gRNA comprises one or more nucleotide residues that comprise a phosphorothioate. In some embodiments, the first and/or the second gRNA comprises one or more nucleotide residues that comprise a thioPACE moiety.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is selected from the group consisting of an embryonic stem cell (ESC), an induced pluripotent stem cell (iPSC), a mesenchymal stem cell, or a tissue-specific stem cell.

Aspects of the present disclosure provide methods comprising a) contacting a cell with (i) a first gRNA comprising a first targeting domain that binds to a first target sequence, and (ii) an RNA-guided nuclease that binds the first gRNA, thus forming a first ribonucleoprotein (RNP) complex under conditions suitable for the first gRNA of (i) to form and/or maintain the first RNP complex with the RNA-guided nuclease of (ii) and for the RNP complex to bind the first target sequence in the genome of the cell; and b) contacting the cell with (iii) a second gRNA comprising a second targeting domain that binds to a second target sequence; and (iv) an RNA-guided nuclease that binds the second gRNA, thus forming a second ribonucleoprotein (RNP) complex under conditions suitable for the second gRNA of (iii) to form and/or maintain the second RNP complex with the RNA-guided nuclease of (iv) and for the second RNP complex to bind a second target sequence in the genome of the cell, wherein steps (a) and (b) are performed sequentially in temporal proximity, separated by a time interval, wherein the first targeting domain is different from the second targeting domain.

In some embodiments, the genetic modification of the first target sequence consists of an insertion or deletion at or immediately proximal to a site cut by the RNA-guided nuclease when bound to the first gRNA; and/or the genetic modification of the second target sequence consists of an insertion or deletion immediately proximal to a site cut by the RNA-guided nuclease when bound to the second gRNA. In some embodiments, the method produces a subpopulation of translocation product cells, wherein each cell of the subpopulation comprises a translocation product comprising a portion of the genome comprising the first target sequence, a portion of the genome comprising the second target sequence, or both. In some embodiments, the method produces fewer translocation product cells as compared to a method comprising contacting a cell with the second gRNA of (iii) prior to contacting the cell with the first gRNA of (i). In some embodiments, the method produces at least 10% fewer translocation product cells as compared to a method comprising contacting the cell with the second gRNA of (iii) prior to contacting the cell with the first gRNA of (i). In some embodiments, the method produces fewer translocation product cells as compared to a method comprising contacting the cell with the first gRNA of (i) and the second gRNA of (iii) at substantially the same time. In some embodiments, the method produces at least 10% fewer translocation product cells as compared to a method comprising contacting the cell with the first gRNA of (i) and the second gRNA of (iii) at substantially the same time.

In some embodiments, binding of the first RNP complex comprising (i) and (ii) to the first target sequence results in a genetic modification generated by a Non-Homologous End Joining (NHEJ) event. In some embodiments, binding of the RNP complex comprising (i) and (ii) to the first target sequence produces a fast-resolving double strand break. In some embodiments, binding of the second RNP complex comprising (iii) and (iv) to the second target sequence results in a genetic modification generated by a microhomology-mediated end joining (MMEJ) event. In some embodiments, binding of the second RNP complex comprising (iii) and (iv) to the second target sequence produces a slow-resolving double strand break.

In some embodiments, the first target sequence is present in a first gene, a transcriptional control element operably linked thereto, or a portion of the gene and transcriptional control element. In some embodiments, the genomic modification of the first target sequence results in reduced or eliminated expression of the product encoded by the first gene, or expression of a variant of the product expressed by wild-type cells of the same cell type that do not harbor a genomic modification in the first target sequence.

In some embodiments, the first gene encodes a first lineage-specific cell-surface antigen. In some embodiments, the first lineage-specific cell-surface antigen is selected from the group consisting of CD33, CD19, CD123, CLL-1, CD30, CD5, CD6, CD7, CD38, and BCMA. In some embodiments, the second target sequence is present in a second gene, a transcriptional control element operably linked thereto, or a portion of the gene and transcriptional control element. In some embodiments, the genomic modification of the second target sequence results in reduced or eliminated expression of the product encoded by the second gene, or expression of a variant of the product expressed by wild-type cells of the same cell type that do not harbor a genomic modification in the second target sequence. In some embodiments, the second gene encodes a second lineage-specific cell-surface antigen. In some embodiments, the second lineage-specific cell-surface antigen is selected from the group consisting of CD33, CD19, CD123, CLL-1, CD30, CD5, CD6, CD7, CD38, and BCMA. In some embodiments, the first lineage-specific cell-surface antigen is CD33. In some embodiments, the second lineage-specific cell-surface antigen is CD19, CD5, or CLL-1. In some embodiments, the first lineage-specific cell-surface antigen is CD5. In some embodiments, the second lineage-specific cell-surface antigen is CD33.

In some embodiments, the first lineage-specific cell-surface antigen is CD33 and the second lineage-specific cell-surface antigen is CLL-1. In some embodiments, the first lineage-specific cell-surface antigen is CLL-1 and the second lineage-specific cell-surface antigen is CD33.

In some embodiments, the time interval between step (b) and step (c) is at least 4, 6, 8, 10, 12, 14, 16, 18, 20, 22, 24, 26, 28, 30, 32, 34, or 36 hours.

In some embodiments, the RNA-guided nuclease of (ii) and/or the RNA-guided nuclease of (iv) is a CRISPR/Cas nuclease. In some embodiments, the CRISPR/Cas nuclease is a Cas9 nuclease. In some embodiments, the CRISPR/Cas nuclease is an spCas nuclease. In some embodiments, the CRISPR/Cas nuclease is an saCas nuclease. In some embodiments, the CRISPR/Cas nuclease is a Cpf1 nuclease. In some embodiments, the RNA-guided nuclease of (ii) is a Cas9 nuclease and the RNA-guided nuclease of (iv) is a Cpf1 nuclease. In some embodiments, the RNA-guided nuclease of (ii) is a Cpf1 nuclease and the RNA-guided nuclease of (iv) is a Cas9 nuclease. In some embodiments, the RNA-guided nuclease of (ii) and the RNA-guided nuclease of (iv) are Cpf1 nucleases. In some embodiments, the RNA-guided nuclease of (ii) and the RNA-guided nuclease of (iv) are Cas9 nucleases.

In some embodiments, the contacting of (a) comprises introducing (i) and (ii) into the cell in the form of a pre-formed ribonucleoprotein (RNP) complex; and/or wherein the contacting of (b) comprises introducing (iii) and (iv) into the cell in the form of a pre-formed ribonucleoprotein (RNP) complex. In some embodiments, the pre-formed ribonucleoprotein (RNP) complex is introduced into the cell via electroporation. In some embodiments, the contacting of (a) comprises introducing (i) and/or (ii) into the cell in the form of a nucleic acid encoding the gRNA of (i) and/or the RNA-guided nuclease of (ii); and/or wherein the contacting of (b) comprises introducing (iii) and/or (iv) into the cell in the form of a nucleic acid encoding the gRNA of (i) and/or the RNA-guided nuclease of (ii).

In some embodiments, the nucleic acid encoding the first gRNA of (i) and/or the RNA-guided nuclease of (ii) is an RNA, preferably an mRNA or an mRNA analog. In some embodiments, the nucleic acid encoding the second gRNA of (iii) and/or the RNA-guided nuclease of (iv) is an RNA, preferably an mRNA or an mRNA analog. In some embodiments, the first gRNA and/or the second gRNA comprises one or more nucleotide residues that are chemically modified. In some embodiments, the first and/or the second gRNA comprises one or more nucleotide residues that comprise a 2′O-methyl moiety. In some embodiments, the first and/or the second gRNA comprises one or more nucleotide residues that comprise a phosphorothioate. In some embodiments, the first and/or the second gRNA comprises one or more nucleotide residues that comprise a thioPACE moiety.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is selected from the group consisting of an embryonic stem cell (ESC), an induced pluripotent stem cell (iPSC), a mesenchymal stem cell, or a tissue-specific stem cell.

Aspects of the present disclosure provide genetically engineered cells, or descendants thereof, produced by any of the methods described herein. In some aspects, the present disclosure provides cell populations comprising a plurality of cells obtained by or obtainable by any of the methods described herein. In some aspects, the present disclosure provides pharmaceutical compositions comprising any of the cells, or a descendants thereof, or any of the cell populations described herein.

Aspects of the present disclosure provide methods comprising administering to a subject in need thereof any of the cells, or descendants thereof, described herein, or any of the cell populations or pharmaceutical compositions described herein. In some embodiments, the cell or descendant thereof or the cells of the cell population comprise a modification in a first gene relative to a wild-type counterpart cell and a modification to a second gene relative to a wild-type counterpart cell. In some embodiments, the method further comprises administering to the subject a therapeutically effective amount of at least one agent that targets a product encoded by the first gene or a wild-type copy thereof, wherein the agent comprises an antigen binding fragment that binds the product encoded by the first gene or a wild-type copy thereof. In some embodiments, administration of the at least one agent targeting the product encoded by the first gene or a wildtype copy thereof occurs simultaneously or in temporal proximity with administration of any of the cells, or a descendants thereof, or any of the cell populations described herein.

In some embodiments, administration of the at least one agent targeting the product encoded by the first gene or a wildtype copy thereof occurs after administration of any of the cells, or a descendants thereof, or any of the cell populations described herein. In some embodiments, administration of the at least one agent targeting the product encoded by the first gene or a wildtype copy thereof occurs before administration of any of the cells, or a descendants thereof, or any of the cell populations described herein.

In some embodiments, the method further comprises administering to the subject a therapeutically effective amount of at least one agent that targets a product encoded by the second gene or a wildtype copy thereof, wherein the agent comprises an antigen binding fragment that binds the product encoded by the second gene or a wildtype copy thereof.

In some embodiments, administration of the at least one agent targeting the product encoded by the second gene or a wildtype copy thereof occurs simultaneously or in temporal proximity with administration of any of the cells, or a descendants thereof, or any of the cell populations described herein. In some embodiments, administration of the at least one agent targeting the product encoded by the second gene or a wildtype copy thereof occurs after administration of any of the cells, or a descendants thereof, or any of the cell populations described herein. In some embodiments, administration of the at least one agent targeting the product encoded by the second gene or a wildtype copy thereof occurs before administration of any of the cells, or a descendants thereof, or any of the cell populations described herein. In some embodiments, administration of the at least one agent targeting the product encoded by the second gene or a wildtype copy thereof occurs simultaneously or in temporal proximity with administration of the at least one agent targeting the product encoded by the first gene or a wildtype copy thereof.

In some embodiments, administration of the at least one agent targeting the product encoded by the second gene or a wildtype copy thereof occurs after administration of the at least one agent targeting the product encoded by the first gene or a wildtype copy thereof. In some embodiments, administration of the at least one agent targeting the product encoded by the second gene or a wildtype copy thereof occurs before administration of the at least one agent targeting the product encoded by the first gene or a wildtype copy thereof.

In some embodiments, the agent that targets a product encoded by the first gene or a wildtype copy thereof and/or the agent that targets a product encoded by the second gene or a wildtype copy thereof is cytotoxic agent. In some embodiments, the cytotoxic agent is an antibody-drug conjugate or an immune effector cell expressing a chimeric antigen receptor (CAR).

In some embodiments, the subject has a disease associated with cells expressing the modified gene or a wildtype copy thereof. In some embodiments, the subject has a cancer associated with cancer stem cells. In some embodiments, the subject has a hematopoietic malignancy. In some embodiments, the subject has an autoimmune disease.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows a schematic of an exemplary experimental design in which cells (e.g., CD34+ cells) are thawed and incubated for 40 hours, then electroporated with a first gRNA and CRISPR-Cas nuclease, referred to as “EP1.” After 30 hours, the cells are electroporated with a second gRNA and CRISPR-Cas nuclease, referred to as “EP2.” Then, the cells are harvested to assess on-targeting editing as well as the presence of translocation products, for example using both qualitative and quantitative translocation assays.

FIGS. 2A and 2B show viability and editing efficiency measured in CD34+ HSCs based on the experimental design shown in FIG. 1. Groups of cells were electroporated simultaneously with gRNAs and a Cas9 nuclease or sequentially with a first gRNA and Cas9 nuclease followed by a second gRNA and Cas9 nuclease, as shown in FIG. 1, or mock electroporated. FIG. 2A shows percent viability analysis at the indicated time points after the first electroporation (“first zap”). FIG. 2B shows percent on-targeting editing efficiency of CD19 or CD33. “Si CD33+CD19” corresponds to cells that were electroporated simultaneously with a gRNA targeting CD33 and a gRNA targeting CD19 and Cas9 nuclease. “Se CD33>CD19” corresponds to cells that were electroporated sequentially with a first gRNA targeting CD33 and Cas9 nuclease followed by a second gRNA targeting CD19 and Cas9 nuclease. “Se CD19>CD33” corresponds to cells that were electroporated sequentially with a first gRNA targeting CD19 and Cas9 nuclease followed by a second gRNA targeting CD33 and Cas9 nuclease. Alternatively, cells were electroporated with a gRNA targeting CD19 or CD33 and Cas9 nuclease and assessed at 30 hrs or 60 hrs. The percent on-target editing efficiency for each group of cell is shown above each column. In FIG. 2B, for each editing scheme, the left column corresponds to CD19 editing (indicated with an asterisks), and the right column corresponds to CD33 editing.

FIG. 3 shows a schematic of the possible translocation products produced by DNA repair events between the double strand breaks produced by the genetic editing at two genomic loci. On left, the chromosomal region encoding CD19 and a chromosomal region encoding CD33 are shown. The location of the gRNA targeting the respective target is indicated with a triangle. The location of primer pairs spanning the targeted region are shown with arrows and labeled as 3 and 2 for CD19 and 5 and 8 for CD33. On the right, potential translocation products are shown, including acentric, dicentric, and balanced, showing the primer pair used to identify each of the products.

FIG. 4A-4C show translocation analysis and editing efficiency measured in CD34+ HSCs based on the experimental design shown in FIG. 1. FIG. 4A shows results of a qualitative translocation analysis with the target normalized to HPRT. FIG. 4B shows the percentage of translocation species normalized to a reference. For translocation analysis, for each group of cells, the left column corresponds to translocation products detected using primers 5 and 2 (dicentric translocation products), and the right column corresponds to translocation products detected using primers 8 and 2 (balanced translocation products).

FIG. 4C shows the percentage on-targeting editing efficiency of CD19 or CD33. “Si CD33+CD19” corresponds to cells that were electroporated simultaneously with a gRNA targeting CD33 and a gRNA targeting CD19 and Cas9 nuclease. “Se CD33>CD19” corresponds to cells that were electroporated sequentially with a first gRNA targeting CD33 and Cas9 nuclease followed by a second gRNA targeting CD19 and Cas9 nuclease. “Se CD19>CD33” corresponds to cells that were electroporated sequentially with a first gRNA targeting CD19 and Cas9 nuclease followed by a second gRNA targeting CD33 and Cas9 nuclease.

FIGS. 5A and 5B show allele frequencies of CD33 and CD19 genetic editing. FIG. 5A shows alignments of alleles resulting from CD33 genetic editing with the indicated gRNA, which has a high frequency of −1 indels, indicating Non-homologous End Joining (NHEJ) repair. FIG. 5B shows alignments of alleles resulting from CD19 genetic editing with the indicated gRNA, which has a high frequency of −6 and −9 indels, indicating Microhomology-mediated End Joining (MMEJ) repair.

FIGS. 6A-6C show schematics of predicted scenarios in which the temporal sequence of genetic editing and kinetics of the editing reaction may play roles in the production of translocation products. FIG. 6A show predicted scenarios in which the first gRNA and second gRNA are delivered to a cell simultaneously (left) or sequentially (right). On the left, delivery of a first ribonucleoprotein complex (RNP 1) comprising a first gRNA and CRISPR/Cas nuclease and a second RNP complex (RNP 2) comprising a second gRNA and CRISPR/Cas nuclease results in two double strand breaks (DSB), which are repaired with different kinetics, producing indels at the first target (target 1) and second target (target 2), as well as potential trans paring between the two DSB. On the right, delivery of a first ribonucleoprotein complex (RNP 1) comprising a first gRNA and CRISPR/Cas nuclease results in a double strand break (DSB), which is repaired producing indels at the first target (target 1), followed by kinetic distancing of delivery of a second RNP complex (RNP 2) comprising a second gRNA and CRISPR/Cas nuclease that results in a double strand break (DSB) and repair at the second target (target 2). FIG. 6B shows sequential editing using a first gRNA that targets CD33 and a second gRNA that targets CD19. The DSB generated with the CD33 gRNA is thought to be repaired by NHEJ and largely complete by 30 hours post electroporation, the time at which the gRNA targeting CD19 is delivered. FIG. 6C shows sequential editing using a first gRNA that targets CD19 and a second gRNA that targets CD33. The DSB generated with the CD19 gRNA is thought to be repaired by MMEJ and largely not complete by 30 hours post electroporation, the time at which the gRNA targeting CD33 is delivered, allowing for potential microhomology trans paring between the two DSB. The gRNA sequences shown below schematics in FIGS. 6B and 6B show the expected cuts and potential translocation scenarios based on microhomology pairing (e.g., GGT-pairing).

FIGS. 7A and 7B show an exemplary experimental design for assessing persistence of editing and long-term reconstitution of simultaneously or sequentially edited cells. FIG. 7A shows a schematic of an exemplary experimental design in which cells (e.g., CD34+ cells) are thawed and incubated for 40 hours, then electroporated with a first gRNA and CRISPR-Cas nuclease, referred to as “EP1.” After 30 hours, the cells are electroporated with a second gRNA and CRISPR-Cas nuclease, referred to as “EP2.” Then, the cells are administered to immunodeficient mice (e.g., NOD-scid ILRgamma^{null} (“NSG™”) mice) that had been treated with 200 centrigray (cGy) of radiation. FIG. 7B shows experimental groups of cells: group 1 corresponds to a control group that received PBS (“PBS Ctrl”); group 2 corresponds to a control group that was not electroporated (“No EP”); group 3 corresponds to a control group that was simultaneously electroporated with a control gRNA (“Si-gCtrl”); group 4 corresponds to a control group that was sequentially electroporated with a control gRNA (“Se-gCtrl”); group 5 corresponds to cells that were sequentially electroporated with a gRNA targeting CD33 followed by a control gRNA (“gCtrl”); group 6 corresponds to cells that were sequentially electroporated with a gRNA targeting CD5 followed by a control gRNA (“gCtrl”); group 7 corresponds to cells that were simultaneously electroporated with a gRNA targeting CD33 and a gRNA targeting CD5 with a low concentration (15 g) of Cas9 nuclease (“SiLoCas9”); group 8 corresponds to cells that were simultaneously electroporated with a gRNA targeting CD33 and a gRNA targeting CD5 with a high concentration (30 g) of Cas9 nuclease (“SiHiCas9”); group 9 corresponds to cells that were sequentially electroporated with a gRNA targeting CD33 followed by electroporation with a gRNA targeting CD5; and group 10 corresponds to cells that were simultaneously electroporated with a gRNA targeting CD5 followed by electroporation with a gRNA targeting CD33.

FIGS. 8A and 8B show viability and editing efficiency measured in CD34+ HSCs prior to administration to NGG™ mice based on the experimental design shown in FIG. 12A. FIG. 8A shows precent viability analysis of cells at the indicated time (hours) after electroporation 1 (EP1). The experimental groups are shown in the x-axis. FIG. 8B shows percent editing efficiency of cells prior to administration to NGG™ mice. For each experimental group shown in the x-axis, the left column corresponds to editing of CD33, and the right column corresponds to editing of CD5. For each editing scheme, the left column corresponds to CD33 editing (indicated with an asterisks), and the right column corresponds to CD5.

FIG. 9 shows the percentage on-target translocation products (as normalized to chromosome 19 (Ch19)) detected in cells prior to administration to NGG™ mice based on the experimental design shown in FIG. 7A. For each experimental group shown in the x-axis, the stacked columns correspond, from top to bottom, acentric translocation, balanced B, balanced A, and dicentric translocation. The right panel shows a schematic of each of the four distinct translocation species. The location of forward and reverse primers used to assess translocation species are indicated by arrows.

FIG. 10 shows the percentage on-target translocation products (as normalized to chromosome 19 (Ch19)) in input and output samples from mouse bone marrow, based on the experimental design shown in FIG. 12A. For each column, the first number corresponds to the group in FIG. 7B, and the second number corresponds to the individual animal.

FIG. 11 shows the percentage of human bone marrow chimerism 16 weeks after administration of edited CD34+ cells into NSG™ mice based on the experimental design shown in FIG. 7A. The results demonstrate that CD34+ cell fitness was not affected by Cas9 multiplexing electroporation or CD5 editing.

FIGS. 12A-12C show the percentages of specific blood cell types (as a proportion of hCD45+ cells) 16 weeks after administration of edited CD34+ cells into NSG™ mice based on the experimental design shown in FIG. 7A. FIG. 12A shows the percentage of CD19+ cells (B cells) for the indicated groups of cells. FIG. 12B shows the percentage of CD3+ cells (T cells) for the indicated groups of cells. FIG. 12C shows the percentage of CD33+ cells (myeloid cells) for the indicated groups of cells. The results show that B and T cell lineages are not affected by multiplex gene editing of CD33 and CD5 (sequentially or simultaneously), while the percentage of myeloid-lineage cells (hCD33+) is low due to loss of CD33 by targeting CD33 gene editing.

FIGS. 13A-13C show the percentages of specific T cell types in the thymi of mice at the 16 week time point after administration of edited CD34+ cells into NSG™ mice based on the experimental design shown in FIG. 12A. FIG. 13A shows the percentage of CD3+ cells (as a proportion of hCD45+ cells). FIG. 13A shows the percentage of CD4+ cells (as a proportion of CD3+ cells). FIG. 13A shows the percentage of CD8+ cells (as a proportion of CD3+ cells)

FIG. 14 shows percentage viability analysis of CD34+ HSCs at the indicated time points after the first electroporation (“first zap”). “Si Cas9+Cpf1” corresponds to cells that were electroporated simultaneously with a first and second gRNA and Cas9 and Cpf1 nucleases. “Se Cas9>Cpf1” corresponds to cells that were electroporated sequentially with a first gRNA targeting CD33 and Cas9 nuclease followed by a second gRNA targeting CD19 and Cpf1 nuclease. “Se Cpf1>Cas9” corresponds to cells that were electroporated sequentially with a first gRNA targeting CD19 and Cpf1 nuclease followed by a second gRNA targeting CD33 and Cas9 nuclease. Alternatively, cells were electroporated with a gRNA targeting CD33 and Cas9 nuclease or a gRNA targeting CD19 and Cpf1 nuclease. Control cells were either not electroporated (No EP) or mock electroporated (Mock EP).

FIGS. 15A and 15B show editing efficiency measured in CD34+ HSCs at the indicated time (hours) after electroporation 1 (EP1). The experimental groups are shown in the x-axis. For each experimental group shown in the x-axis, the left column corresponds to editing of CD33 (indicated with asterisks), and the right column corresponds to editing of CD19.

FIG. 16 shows results of a translocation analysis with the target normalized to HPRT as a reference. The electroporation conditions of the groups of cells are shown on the x-axis along with the primer pairs used to assess translocation products.

FIG. 17 shows a schematic of an exemplary experimental design for multiplexed editing (bottom panels) as compared to single target-edited cells (i.e., non-multiplex, top panels). For multiplexed editing, cells (e.g., CD34+ cells) are thawed and cultured in Serum-free Expansion Media (SFEM) supplemented with cytokines for 24 hours, then electroporated with a first gRNA targeting CD33 and a CRISPR-Cas nuclease, referred to as “EP1.” The cells are incubated for 30 hours and then electroporated with a second gRNA targeting CLL-1 and a CRISPR-Cas nuclease, referred to as “EP2.” After 63 hours, the cells are harvested, sorted using fluorescence activated cell sorting (FACS), and subjected to sequencing.

FIGS. 18A-18C show the CD33 editing frequency outcomes in various cell types derived from donors. FIG. 18A shows the percentage on-target CD33 editing efficiency in the indicated cell types from each donor. FIGS. 18B and 18C show CD33 indel analysis represented as the percentage editing frequency in cells derived from donors SD01000510 and B01001335, respectively. “Seq CD33:CLL1” corresponds to cells that were sequentially electroporated with a ribonucleoprotein (RNP) complexes comprising a first gRNA targeting CD33 and a CRISPR Cas9 nuclease followed electroporation with RNP complexes comprising a second gRNA targeting CLL-1 and a CRISPR Cas9 nuclease. “LT-HSC” corresponds to long-term hematopoietic stem cells. “CMP” corresponds to common myeloid progenitor stem cells. “MPP” corresponds to multi-potent progenitor cells. “MLP” corresponds to multi-lymphoid progenitor cells. “CD49f” corresponds to hematopoietic stem cells purified using a CD49f antibody. For each donor or indel position, the columns correspond to Seq CD33:CLL1, LT-HSC, CMP, MPP, MLP, and CD49f.

FIGS. 19A-19C show the CLL-1 editing frequency in various cell types derived from donors. FIG. 19A shows the percentage on-target CLL-1 editing efficiency in the indicated cell types from each donor. FIGS. 19B and 19C show CLL-1 indel analysis represented as the percent editing frequency on cells derived from donors SD01000510 and B01001335, respectively. “Seq CD33:CLL-1” corresponds to cells that were sequentially electroporated with a first gRNA targeting CD33 and a CRISPR Cas9 nuclease followed by electroporation with a second gRNA targeting CLL-1 and a CRISPR-Cas nuclease. “LT-HSC” corresponds to long-term hematopoietic stem cells. “CMP” corresponds to common myeloid progenitor cells. “MPP” corresponds to multi-potent progenitor cells. “MLP” corresponds to multi-lymphoid progenitor cells. “CD49f” corresponds to hematopoietic stem cells purified using a CD49f antibody. For each donor or indel position, the columns correspond to Seq CD33:CLL-1, LT-HSC, CMP, MPP, MLP, and CD49f.

FIG. 20 shows viability of the indicated cell types derived from donors following multiplexed editing. “LT-HSC” corresponds to long-term hematopoietic stem cells. “ST-HSC” corresponds to short-term hematopoietic stem cells. “CMP” corresponds to common myeloid progenitor stem cells. “CD49f” corresponds to hematopoietic stem cells purified using a CD49f antibody. “MLP” corresponds to multi-lymphoid progenitor cells. “MPP” corresponds to multi-potent progenitor cells.

FIGS. 21A-21D show CD33 editing and expression analysis in HL60 cells following multiplexed editing. FIG. 21A shows the percentage editing frequency of CD33 as determined by TIDE analysis at the indicated time points. FIG. 21B shows CD33 transcript expression by RT-qPCR in cells electroporated with a gRNA targeting CD33 (g811) or a control gRNA (gCtrl) at the indicated time points following electroporation (days in culture). The CD33 transcript expression is presented as a percent of expression at day 0. FIG. 21C shows CD33 surface expression as assessed by flow cytometric analysis over the indicated time points. FIG. 21D shows a schematic of the CD33 transcript and the location of representative primers used for RT-qPCR analysis. “TIDE” refers to Tracking of Indels by DEcomposition. “RT-qPCR” refers to real-time quantitative polymerase chain reaction. TC1 refers to Time Course 1; and TC2 refers to Time Course 2.

FIGS. 22A-22D show CLL-1 editing and expression analysis in HL60 cells following multiplexed editing. FIG. 22A shows the percentage editing frequency of CLL-1 as determined by TIDE analysis at the indicated time points. FIG. 22B shows CLL-1 transcript expression by RT-qPCR in cells electroporated with a gRNA targeting CLL-1 (g6) or a control gRNA (gCtrl) at the indicated time points following electroporation. FIG. 22C shows CLL-1 surface expression as assessed by flow cytometric analysis at the indicated time points. FIG. 22D shows a schematic of the CLL-1 transcript and the location of representative primers used for RT-qPCR analysis.

FIG. 23 shows a schematic of an exemplary experimental design for sequential multiplexed editing. Cells (e.g., CD34+ cells) are thawed for 40 hours and then sequentially electroporated with ribonucleoprotein complexes (RNPs) comprising gRNAs and CRISPR-Cas nuclease, referred to as “EP1” on day 2 (D2) and “EP2” on day 3 (D3). Twenty-six hours following EP2, the cells are subjected to myeloid differentiation culture conditions for 14 days, including cell counting/splitting on days 8 (D8), 11 (D11), and 18 (D18) prior to phenotypic and functional characterizations (e.g., flow cytometry, phagocytosis, and cytokine release assays).

FIGS. 24A and 24B show growth rate analysis during cell differentiation at the indicated time points. FIG. 24A shows growth rate as the viable cell number during granulocyte differentiation. FIG. 24B shows growth rate as the viable cell number during monocyte differentiation. “Mock>Mock” refers to cells that underwent sequential mock electroporation. “SeqCD33>CLL-1” corresponds to cells that were sequentially electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 25A and 25B show the effects of sequential multiplexed editing on granulocyte differentiation in hematopoietic stem and progenitor cells (HSPCs). FIG. 25A shows the percentage of CD15+ cells at the indicated time points. FIG. 25B shows the percentage of CD11b+ cells at the indicated time points. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 26A and 26B show the effects of sequential multiplexed editing on monocyte differentiation in hematopoietic stem and progenitor cells (HSPCs). FIG. 26A shows the percentage of CD14+ cells at the indicated time points. FIG. 26B shows the percentage of CD11b+ cells at the indicated time points. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 27A and 27B show CLL-1 editing frequency maintained throughout myeloid differentiation at the indicated time pointes. FIG. 27A shows the percentage editing frequency of CLL-1 throughout granulocyte differentiations. FIG. 27B shows the percentage editing frequency of CLL-1 throughout monocyte differentiation. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 28A and 28B show CD33 editing frequency maintained throughout myeloid differentiation at the indicated time points. FIG. 28A shows the percentage editing frequency of CD33 throughout granulocyte differentiation. FIG. 28B shows the percentage editing frequency of CD33 throughout monocyte differentiation. “SeqCD33>CLL-1” corresponds to cells that underwent EP with a first gRNA targeting CD33 and a CRISPR-Cas nuclease followed by EP with a second gRNA targeting CLL-1 and a CRISPR-Cas nuclease. “SeqCLL1>CD33” corresponds to cells that underwent EP with a first gRNA targeting CLL-land a CRISPR-Cas nuclease followed by EP with a second gRNA targeting CD33 and a CRISPR-Cas nuclease.

FIGS. 29A-29D show the effects of multiplexed editing on CLL-1 protein expression as assessed by flow cytometry analyses. FIGS. 29A and 29C show CLL-1 expression in granulocytes at the indicated time points. FIGS. 29B and 29D show CLL-1 expression in monocytes at the indicated time points. Electroporation with gRNAs and CRISPR-Cas nuclease occurred on days 2 and 3, as indicated by arrows. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 30A-30D show the effects of multiplexed editing on CD33 protein expression as assessed by flow cytometry analysis. FIGS. 30A and 30C show CD33 expression in granulocytes at the indicated time points. FIGS. 30B and 30D show CD33 expression in monocytes at the indicated time points. Electroporation with gRNAs and CRISPR-Cas nuclease occurred on days 2 and 3, as indicated by arrows. Mock>Mock” refers to cells that underwent sequential mock electroporation. “SeqCD33>CLL1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 31A and 31B show flow cytometry analysis of CLL-1 and CD33 expression in differentiated myeloid cells on day 18 following multiplexed editing. FIG. 31A shows CLL-1 and CD33 expression analysis in granulocytes. FIG. 31B shows CLL-1 and CD33 expression analysis in monocytes. The segments of each individual bar of the graph correspond, from top to bottom, to CLL-1+CD33+, CLL-1+CD33−, CLL-1-CD33+, and CLL-1−CD33−. The oval over the right-most two bars indicates the percentage of cells that were deficient in CLL-1 and CD33 expression. “SeqCD33:CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1:CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIGS. 32A and 32B show the phagocytic ability of differentiated myeloid cells following multiplexed editing. Phagocytic ability was analyzed as the percent pHrodo+ cells that were subjected to pHrodo+E. coli cells or pHrodo+E. coli and cytochalasin D (“CytoD”). FIG. 32A shows phagocytosis as percent pHrodo+ cells in granulocytes. FIG. 32B shows phagocytosis as percent pHrodo+ cells in monocytes. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease.

FIG. 33 shows a schematic of an exemplary experimental design to assess lineage differentiation. Freshly electroporated cells are mixed in a methylcellulose-based differentiation medium (MethoCult™) and then plated. After incubating for 14 days, cells are imaged and scored. “BFU-E” refers to burst-forming unit-erythroid. “CFU-G/M/GM” refers to colony-forming units—granulocyte/macrophage.” “CFU-GEMM” refers to colony-forming units of multipotential myeloid progenitor cells that generate granulocyte, erythroid, macrophage, and megakaryocytes.

FIGS. 34A and 34B show colony forming unit (CFU) analysis of multiplex edited cells. FIG. 34A shows the number of colonies formed when cells were plated at a dilution of 200 cells/well. FIG. 34B shows the number of colonies formed when cells were plated at a dilution of 300 cells/well. “Mock>Mock” refers to cells that underwent sequential mock electroporation. “SeqCD33>CLL-1” corresponds to cells that were electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease. “GEMM” refers to multipotential myeloid progenitor cells that generate granulocyte, erythroid, macrophage, and megakaryocytes. “G/M/GM” refers to granulocyte, macrophage. “BFU-E” refers to burst-forming unit—erythroid. For each editing condition, the segments of the bar correspond, from top to bottom, GEMM, G/M/GM, and BFU-E.

FIGS. 35A and 35B show colony distribution analysis of CFUs formed by multiplex edited cells. FIG. 35A shows the distribution of CFUs formed by cells plated at a dilution of 200 cells/well. FIG. 35B shows the distribution of CFUs formed by cells plated a dilution of 300 cells/well. “SeqCD33>CLL-1” corresponds to cells that underwent EP with a first gRNA targeting CD33 and a CRISPR-Cas nuclease followed by EP with a second gRNA targeting CLL-1 and a CRISPR-Cas nuclease. “SeqCLL-1>CD33” corresponds to cells that underwent EP with a first gRNA targeting CLL-1 and a CRISPR-Cas nuclease followed by EP with a second gRNA targeting CD33 and a CRISPR-Cas nuclease. “Mock>Mock” refers to cells that underwent sequential mock electroporation. “CFU” refers to colony-forming units. “GE MM” refers to granulocyte, erythroid, macrophage, megakaryocyte. “G/M/GM” refers to granulocyte, macrophage. “BFU-E” refers to burst-forming unit—erythroid.

FIGS. 36A and 36B show an exemplary experimental design to assess for long-term engraftment of multiplex edited hematopoietic cells. FIG. 36A shows a schematic of an exemplary multiplex editing and engraftment approach in which cells (e.g., CD34+ cells) are edited in vitro, left panel, and engrafted into NSG™ mice. At 8 weeks post-transplant, blood samples are harvested and assessed by flow cytometric cell sorting (FACS). At 16 weeks post-transplant, blood samples and bone marrow are harvested and assessed by FACs, sequencing (gDNA), and viability. FIG. 36B shows an exemplary list of experimental conditions. “eHSC” refers to embryonic stem cells. “N” refers to the number of mice in the indicated treatment group. “SeqCD33>CLL-1” corresponds to cells that underwent EP with a first gRNA targeting CD33 and a CRISPR-Cas nuclease followed by EP with a second gRNA targeting CLL-1 and a CRISPR-Cas nuclease. “SeqCLL-1>CD33” corresponds to cells that underwent EP with a first gRNA targeting CLL-1 and a CRISPR-Cas nuclease followed by EP with a second gRNA targeting CD33 and a CRISPR-Cas nuclease. “SiHi CD33+CLL-1” refers to cells that were electroporated simultaneously with a gRNA targeting CD33 and a gRNA targeting CLL-1 and a high concentration of a CRISPR-Cas nuclease (30 μg Cas nuclease per gRNA used, 60 μg Cas nuclease total). “Se Mock>Mock” refers to cells that underwent sequential mock electroporation. “No EP” refers to cells that were not electroporated.

FIG. 37 is a table showing cell number and viability of cells prior to cryopreservation (before cryo) and after thawing (post thaw).

FIG. 38 shows analysis of bone marrow (BM) chimerism 16 weeks-post engraftment of multiplexed edited cells. “No EP” refers to cells that were not electroporated. “SeqCD33>CLL-1” corresponds to cells that were sequentially electroporated with a first gRNA targeting CD33 and a CRISPR-Cas nuclease followed by electroporation with a second gRNA targeting CLL-1 and a CRISPR-Cas nuclease. “SeqCLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease. “SiHi CD33+CLL-1” refers to cells that were electroporated simultaneously with a gRNA targeting CD33 and a gRNA targeting CLL-1 with a high concentration of Cas9 nuclease.

FIGS. 39A-39H show flow cytometry analyses of myeloid and lymphoid lineage cells harvested from mouse models following engraftment. FIG. 39A shows T-lymphocytes (CD3+/hCD45+). FIG. 39B shows monocytes (CD14+/hCD45+). FIG. 39C shows neutrophils (CD15+/hCD45+). FIG. 39D shows mast/basophil cells (CD203+/hCD45+). FIG. 39E shows B-lymphocytes (CD19+/hCD45+). FIG. 39F shows hematopoietic stem and progenitor cells (HSPCs, CD34+/hCD45+). FIG. 39G shows classical dendritic cells (cDCs, cDC/hCD45+). FIG. 39H shows plasmacytoid dendritic cells (pCDs, pDC/hCD45+). “Sc CD33>CLL-1” corresponds to cells that were sequentially electroporated with a first gRNA targeting CD33 and a CRISPR-Cas nuclease followed by electroporation with a second gRNA targeting CLL-1 and a CRISPR-Cas nuclease. “Sc CLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease. “SiHi CD33+CLL-1” refers to cells that were electroporated simultaneously with a gRNA targeting CD33 and a gRNA targeting CLL-1 with a high concentration of Cas9 nuclease.

FIGS. 40A-40F show CD33 and CLL-1 protein expression levels in myeloid and lymphoid lineage cells flow cytometry analyses of cells harvested from mice following engraftment. FIG. 40A shows expression in total human CD45+ cells. FIG. 40B shows expression on monocytes (CD14+ cells). FIG. 40C shows neutrophils (CD15+ cells). FIG. 40D shows mast/basophil cells (CD203+ cells). FIG. 40E shows classic dendritic cells (cDCs). FIG. 40F shows plasmacytoid dendritic cells (pDCs). “No EP” refers to cells that were not electroporated. “Sc CD33>CLL-1” corresponds to cells that were sequentially electroporated with a first gRNA targeting CD33 and a CRISPR-Cas nuclease followed by electroporation with a second gRNA targeting CLL-1 and a CRISPR-Cas nuclease. “Sc CLL-1>CD33” corresponds to cells that were sequentially electroporated with a first gRNA targeting CLL-1 and a CRISPR-Cas nuclease followed by electroporation with a second gRNA targeting CD33 and a CRISPR-Cas nuclease. “SiHi CD33+CLL-1” refers to cells that were electroporated simultaneously with a gRNA targeting CD33 and a gRNA targeting CLL-1 with a high concentration of Cas9 nuclease. For each editing condition, the segments of the bar correspond, from top to bottom, to CD33-CLL-1−, CD33-CLL-1+, CD33+, CLL-1−, and CD33+CLL-1+.

FIGS. 41A and 41B show CD33 and CLL-1 protein expression levels in CD14+ cells harvested from mice 16 weeks post-engraftment as assessed by flow cytometry. FIG. 41A shows no electroporation control cells, with 91.5% of cells being CD33+ and CLL-1+. FIG. 41B shows cells that were sequentially electroporated with a first gRNA targeting CD33 and a CRISPR-Cas nuclease followed by electroporation with a second gRNA targeting CLL-1 and a CRISPR-Cas nuclease (SeqCD33>CLL-1), with 98.3% of cells being CD33- and CLL-1-negative.

FIG. 42 shows on-target editing as assessed by next generation sequencing (NGS) and the percent editing efficiency at CD33 using the CD33 targeting gRNA (CD33g811; left columns) and at CLL-1 using the CLL-1 targeting gRNA (CLL-1g6, right columns) at the 56-hour time point post electroporation. “SiHi” refers to cells that were simultaneously electroporated with a gRNA targeting CD33, a gRNA targeting CLL-1, and a high concentration of a CRISPR-Cas nuclease (30 μg Cas nuclease per gRNA used, 60 μg Cas nuclease total). On-target editing was assessed by target amplicon sequencing (rhAmpSeq™) “CD33g811>CLL-1” corresponds to cells that were sequentially electroporated with a first gRNA targeting CD33 (g811) and a CRISPR-Cas nuclease followed by electroporation with a second gRNA targeting CLL-1 and a CRISPR-Cas nuclease. “CLL-1>CD33g811” corresponds to cells that were sequentially electroporated with a first gRNA targeting CLL-1 and a CRISPR-Cas nuclease followed by electroporation with a second gRNA targeting CD33 (g811) and a CRISPR-Cas nuclease.

FIGS. 43A and 43B show editing at the indicated time points post-electroporation (EP) as assessed by Inference of CRISPR Edits (ICE) analysis. FIG. 43A shows editing efficiency using the CD33 targeting gRNA, CD33g811, and CLL-1 targeting gRNA, CLL-1g6, at the 30 hour, 50 hour, and 56 hour time points. FIG. 43B shows editing efficiency at the 56 hour time point as reflected in FIG. 43A. “SiHi CD33+CLL-1” refers to cells that were electroporated simultaneously with a gRNA targeting CD33, a gRNA targeting CLL-1, and a high concentration of Cas9 nuclease. “Se CD33>CLL-1” refers to cells that were sequentially electroporated with a first gRNA targeting CD33 and a CRISPR-Cas nuclease followed by electroporation with a second gRNA targeting CLL-1 and a CRISPR-Cas nuclease. “Se CLL-1>CD33” refers to cells that were sequentially electroporated with a first gRNA targeting CLL-1 and a CRISPR-Cas nuclease followed by electroporation with a second gRNA targeting CD33 and a CRISPR-Cas nuclease.

FIG. 44 shows a schematic of exemplary translocation events.

FIG. 45 shows analysis of translocation events in multiplex edited hematopoietic cells at 56 hours-post electroporation with the first gRNA and CRISPR-Cas nuclease, referred to as “EP1.” “For each editing condition, the columns correspond, from left to right, to dicentric, balanced 1, balanced 2, and acentric translocation. “SeMock>Mock” refers to mock electroporated cells. “SiHiCD33+CLL-1” refers to cells that were electroporated simultaneously with a gRNA targeting CD33, a gRNA targeting CLL-1, and a high concentration of Cas9 nuclease.” “Se CD33>CLL-1” refers to cells that were sequentially electroporated with a first gRNA targeting CD33 and a CRISPR-Cas nuclease followed by electroporation with a second gRNA targeting CLL-1 and a CRISPR-Cas nuclease. “Se CLL-1>CD33” refers to cells that were sequentially electroporated with a first gRNA targeting CLL-1 and a CRISPR-Cas nuclease followed by electroporation with a second gRNA targeting CD33 and a CRISPR-Cas nuclease.

FIG. 46 shows editing efficiency of bone marrow (BM) cells at 16 weeks following engraftment as assessed by rhAMP-Seq™. For each editing condition, the left data points correspond to editing efficiency at CD33 using the CD33 targeting gRNA g811 (CD33g811), and the right data points correspond to editing efficiency at CLL-1 using the CLL-1 targeting gRNA g6 (CLL-1g6). The square data point indicates the input sample. “CD33>CLL-1” refers to cells that were sequentially electroporated with a first gRNA targeting CD33 and a CRISPR-Cas nuclease followed by electroporation with a second gRNA targeting CLL-1 and a CRISPR-Cas nuclease. “CLL-1>CD33” refers to cells that were sequentially electroporated with a first gRNA targeting CLL-1 and a CRISPR-Cas nuclease followed by electroporation with a second gRNA targeting CD33 and a CRISPR-Cas nuclease. “CD33+CLL-1” refers to cells that were electroporated simultaneously with a gRNA targeting CD33, a gRNA targeting CLL-1, and a high concentration of Cas9 nuclease.”

FIG. 47 shows analysis of off-target editing events following electroporation of cells with a gRNA targeting CLL-1 (CLL1-g6) and a CRISPR-Cas nuclease determined using CasOFFinder.

FIG. 48 shows analysis of on-target editing frequency across three hematopoietic cell donors HC1, HC2, and HC3 using the CLL-1 targeting gRNA, g6. The editing frequency was determined using both ICE (left column) and hybrid capture (right column). “ICE” refers to Inference of CRISPR Edits analysis.

FIG. 49 shows predicted off-target editing events in the CLL-1 gene as a result of electroporation with a CLL-1-targeting gRNA (g6) and a CRISPR-Cas nuclease.

FIG. 50 shows an exemplary insertion deletion (indel) spectrum following editing using the CLL-1 targeting gRNA g6.

FIG. 51 shows survival of target hematopoietic cells following incubation with immune cells expressing chimeric antigen receptor (CARs) targeting CD33 or CLL-1. The target hematopoietic cells are wildtype (WT), deficient in CD33 (CD33^{Del}), deficient in CLL-1 (CLL-1^{Del}) or deficient in both CD33 and CLL-1 (CD33^{Del}CLL-1^{Del})

FIGS. 52A-52D show cytokine production of differentiated myeloid cells following multiplexed editing (“MPX”) of CD33 and CLL-1 as compared to control, unedited cells (“CTR”). FIG. 52A shows IL-6 production. FIG. 52B shows IL-8 production. FIG. 52C shows TNFα production. FIG. 52D shows MIP-10 production. Cytokine production was assessed at basal level and 24 hours after stimulation with lipopolysaccharide (LPS) or resiquimod (R848). N=3. Data is shown as the mean+/−standard deviation.

FIG. 53 shows the total translocation events as percent translocation in input cells (CD34+′ “input CD34+ cells”) and output xenograft bone marrow cells (“output BM”) from NSG mice at 16 weeks after transplantation with MPX-edited or CTR-edited hHSPCs. “CTR” refers to control cells, and “MPX” refers to multiplexed edited cells. N=12 mice per group.

FIGS. 54A-54D show CD33 and CLL-1 expression on AML patient-derived blasts and leukemic stem cells (LSCs). FIG. 54A shows the percentage of CD33-positive and CLL-1 positive cells (% antigen positivity) by flow cytometry in AML blasts (N=33). FIG. 54B shows the percentage of CD33-positive and CLL-1 positive cells (% antigen positivity) by flow cytometry in AML LSCs (N=27). FIG. 54C shows the antigen density of CD33 and CLL-1 on the cell surface of AML blasts quantified by flow cytometry (N=31). FIG. 54D shows the antigen density of CD33 and CLL-1 on the cell surface of AML LSCs quantified by flow cytometry (N=25).

### DETAILED DESCRIPTION

Use of CRISPR/Cas systems to effect genetic modifications presents a versatile and adaptable platform, however, there are a number of potential risks associated with CRISPR/Cas use in therapeutic applications, such as off-target effects, risk of translocation events, and potential malignancy. When introducing genetic modifications, for example using CRISPR/Cas systems, breaks in the DNA (e.g., double stranded breaks (DSB)) are introduced in the genome of the cell, leading to non-homologous end-joining (NHEJ) repair of the break and insertions or deletions (indels) proximal to the target sequence. This process may lead to frameshifts and inactivation of genes, however it can also result in chromosomal translocations when two chromosomes or fragments of chromosomes are improperly joined. Chromosomal translocation is associated with genomic instability and various types of cancers, for example through the expression of new fusion proteins, expression or misregulation of oncogenes. See, e.g., Brunet et al. Adv. Exp. Med. Biol. (2018) 1044: 15-25; Ghezraoui H, et al. (2014) Mol Cell 55(6):829-842; Bothmer et al. CRISPR Journal (2020) 3(3). Multiplexed genetic editing, in which multiple genetic modifications are introduced into a cell, may have increased potential risk of translocation events, in particular if more than one DNA break (e.g., DSB) are present at the same time or substantially the same time. To minimize or reduce potential adverse effects, mechanisms of regulating the introduction of the genetic modifications, for example to reduce the risk of translocation events, are desired.

Aspects of the present disclosure provide methods of genetic editing that are effective in generating multiple genetic modifications in a cell and reducing the risk of translocation events (e.g., production of translocation products). In some aspects, the methods described herein involve contacting a cell or population of cells (plurality of cells) with a first guide RNA (gRNA) and an RNA-guided nuclease to effect a first genetic modification followed by contacting the cell or population of cells (plurality of cells) with a second gRNA and an RNA-guided nuclease to effect a second genetic modification in the cell, wherein the contacting steps are performed sequentially and separated by a time interval. Also provided herein are cells produced by the methods described herein and methods involving administering any of the genetically engineered cells, or descendants thereof, produced by the methods described herein to a subject.

As will be understood by one of skill in the art, generation of double strand breaks (DSB) in the genome of a cell recruits DNA repair machinery to promote DNA repair at the break site. In general, the DNA repair may proceed through any of several repair pathways, the primary pathways for which are homology directed repair (HDR, also referred to as “homologous recombination”), non-homologous end-joining (“NHEJ,” also referred to as classical non-homologous end-joining (“c-NHEJ”)), and microhomology-mediated end-joining (“MMEJ,” also referred to as alternative end-joining (“alt-EJ”)).

HDR involves the presence of a homologous template that is used to correct the break and is typically considered to result in precise (error-free) repair. The NHEJ mechanism is an efficient yet error-prone repair mechanism that produces insertions and deletions (indels) and does not involve a homologous template. NHEJ involves direct ligation of the ends of the double stranded break and involves the Ku protein that recruits additional NHEJ proteins to the site including the DNA ligase IV complex. The MMEJ repair mechanism involves microhomologies within the ends of the double stranded breaks (typically between 1-25 nucleotides). MMEJ proceeds through a series of steps in which the regions of microhomology are annealed, heterologous “flaps” of nucleotides are removed, followed by fill-in synthesis of the gaps and ligation. This mechanism is error-prone and associated with deletion of nucleotides flanking the DSB. See, e.g., Zaboikin et al. PLos One (2017) 12(1): e0169931; Wang et al. Cell & Bioscience (2017) 7:6; Deriano et al. Ann. Rev. Genet. (2013) 47: 433-455.

The term “mutation” is used herein to refer to a genetic change (e.g., insertion, deletion, inversion, or substitution) in a nucleic acid compared to a reference sequence, e.g., the corresponding sequence of a cell not having such a mutation, or the corresponding wild-type nucleic acid sequence. In some embodiments, the cells produced using the methods described herein comprise more than one mutation (e.g., 2, 3, 4, 5, or more) mutations compared to a reference sequence, e.g., the corresponding sequence of a cell not having such a mutation, or the corresponding wild-type nucleic acid sequence. In some embodiments, a mutation to a gene (e.g., a target gene) results in a loss of expression of a protein encoded by the target gene in a cell harboring the mutation. In some embodiments, a mutation in a gene (e.g., a target gene) results in the expression of a variant form of a protein that is encoded by the target gene.

Some aspects of this disclosure provide compositions and methods for generating the genetically engineered cells described herein, e.g., genetically engineered cells comprising more than one modification in their genome, such as a modification that results in a loss of expression or regulation of a protein(s), and/or or expression of a variant form of a protein(s). Such compositions and methods provided herein include, without limitation, suitable strategies and approaches for genetically engineering cells, e.g., by using RNA-guided nucleases, such as CRISPR/Cas nucleases, and suitable guide RNAs able to bind such RNA-guided nucleases and target them to a suitable target site within the genome of a cell to effect a genomic modification.

In some embodiments, a genetically engineered cell described herein is generated via genome editing technology, which includes any technology capable of introducing targeted changes, also referred to as “edits,” into the genome of a cell. In some embodiments, the genetically engineered cells comprise a plurality of edits in the genome of the cells.

### CRISPR/Cas Systems

Some aspects of this disclosure provide compositions and methods for generating the genetically engineered cells described herein. One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. In some embodiments, the RNA-guided nucleases (e.g., CRISPR/Cas nucleases) used in the methods described herein are capable of producing a double-stranded break in DNA. In some embodiments, the RNA-guided nucleases (e.g., CRISPR/Cas nucleases) used in the methods described herein has reduced nuclease activity. For example, in some embodiments, a suitable RNA-guided nuclease for use in the methods of genetically engineering cells provided herein is a Cas9 nuclease, e.g., an spCas9 or an saCas9 nuclease.

For another example, in some embodiments, a suitable RNA-guided nuclease for use in the methods of genetically engineering cells provided herein is a Cas12 nuclease, e.g., a Cas12a nuclease (also referred to as a “Cpf1 nuclease”). As used herein, a Cpf1 nuclease refers to a polypeptide i) derived from a type II class 2 CRISPR/Cas nuclease the cleaves distal to a PAM site, and ii) capable of, in combination with a suitable gRNA, binds to a target nucleic acid sequence (a target sequence). Exemplary suitable Cas12 nucleases include, without limitation, AsCas12a, FnCas12a, LbCas12a, PaCas12a, other Cas12a orthologs, and Cas12a derivatives, such as the MAD7™ system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816; PCT Publication Nos. WO 2016/166340; WO 2017/155407; WO 2018/083128; WO 2016/205711; WO 2017/035388; WO 2017/184768; WO2019/118516; WO2017/184768; WO 2018/098383; WO 2020/146297; and WO 2020/172502.

The methods described herein involve targeting a first RNA-guided nuclease e.g., a CRISPR/Cas nuclease, for example a Cas9 nuclease or a Cas12a nuclease (e.g., Cpf1) to a suitable target site in the genome of the cell, under conditions suitable for the RNA-guided nuclease to bind the target site and cut the genomic DNA of the cell, followed by targeting a second RNA-guided nuclease e.g., a CRISPR/Cas nuclease, for example a Cas9 nuclease or a Cas12a nuclease (e.g., Cpf1) to a second suitable target site in the genome of the cell, under conditions suitable for the second RNA-guided nuclease to bind the target site and cut the genomic DNA of the cell. In some embodiments, the first RNA-guided nuclease and the second RNA-guided nuclease are of the same type of nuclease, e.g., CRISPR/Cas nucleases, for example, both the first RNA-guided nuclease and the second RNA-guided nuclease are Cas9 nucleases or Cpf1 nucleases. In some embodiments, the first RNA-guided nuclease and the second RNA-guided nuclease are of different types of nucleases, e.g., CRISPR/Cas nucleases, for example, the first RNA-guided nuclease is a Cas9 nuclease and the second RNA-guided nuclease is a Cpf1 nuclease. In some embodiments, the first RNA-guided nuclease and the second RNA-guided nuclease are of different types of nucleases, e.g., CRISPR/Cas nucleases, for example, the first RNA-guided nuclease is a Cpf1 nuclease and the second RNA-guided nuclease is a Cas9 nuclease. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs (i.e., gRNAs) are described in more detail elsewhere herein.

In some embodiments, any of the gRNAs described herein may be complexed with a suitable CRISPR/Cas nuclease. Exemplary suitable nucleases include, for example, Cas12a (Cpf1) nucleases and Cas 9 nucleases.

Various Cas9 nucleases are suitable for use with the gRNAs provided herein to effect genome editing according to aspects of this disclosure, e.g., to create a genomic modification in the CD30 gene. Typically, the CRISPR/Cas nuclease and the gRNA are provided in a form and under conditions suitable for the formation of a nuclease/gRNA complex (e.g., a CRISPR system), which may be referred to as a ribonucleoprotein (RNP) complex, that targets a target site on the genome of the cell. In some embodiments, a CRISPR/Cas nuclease is used that exhibits a desired PAM specificity to target the nuclease/gRNA complex to a desired target site sequence in a genetic loci.

In some embodiments, a nuclease/gRNA complex is formed, e.g., in vitro, and a target cell is contacted with the nuclease/gRNA complex, e.g., via electroporation of the Cas/gRNA complex into the cell. In some embodiments, the cell is contacted with a CRISPR/Cas protein and gRNA separately, and the nuclease/gRNA complex is formed within the cell. In some embodiments, the cell is contacted with a nucleic acid, e.g., a DNA or RNA, encoding the CRISPR/Cas protein, and/or with a nucleic acid encoding the gRNA, or both.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71: 1-9.

In some embodiments, genetically engineered cells as provided herein are generated using a suitable genome editing technology, wherein the genome editing technology is characterized by the use of a Cas12a (Cpf1) nuclease.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus Puniceispirillum, Clostridium cellulolyticum, Clostridium perfringens, Corynebacterium accolens, Corynebacterium diphtheria, Corynebacterium 5 matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71: 1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7™ (from Inscripta).

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

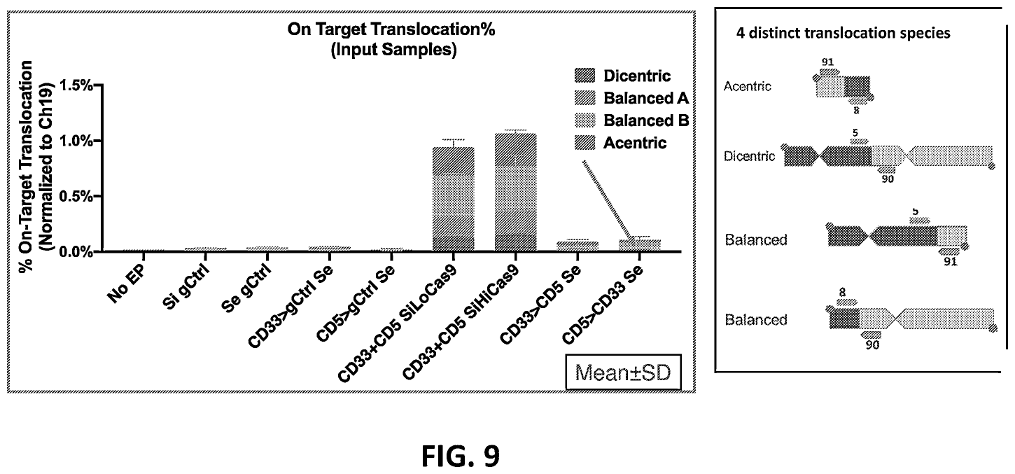

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science (2014) 343(6176): 1247997) and for S. pyogenes Cas9 with a guide RNA (e.g., a synthetic fusion of crRNA and tracrRNA) (Nishimasu et al., Cell (2014) 156:935-949; and Anders et al., Nature (2014) doi: 10.1038/nature13579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, spCas9 and saCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35(8): 789-792.

In some embodiments, a base editor is used to create a genomic modification in a cell. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase.

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a target site in the genome of a cell. In some embodiments, the gRNA effects a modification in the genome of the cell (e.g., insertion, mutation, deletion). Such modifications may result in a loss of expression and/or regulation of a protein encoded by a gene, or expression of a variant form of a gene encoded by a genet that is targeted by the gRNA.

The terms “gRNA” and “guide RNA” are used interchangeably throughout and refer to a nucleic acid that promotes the specific targeting or homing of a gRNA/Cas9 molecule complex to a target nucleic acid. A gRNA can be unimolecular (having a single RNA molecule), sometimes referred to herein as sgRNAs, or modular (comprising more than one, and typically two, separate RNA molecules). A gRNA may bind to a target sequence in the genome of a host cell. The gRNA (e.g., the targeting domain thereof) may be partially or completely complementary to the target sequence. The gRNA may also comprise a “scaffold sequence,” (e.g., a tracrRNA sequence), that recruits a Cas9 molecule to a target sequence bound to a gRNA sequence (e.g., by the targeting domain of the gRNA sequence). The scaffold sequence may comprise at least one stem loop structure and recruits an endonuclease. Exemplary scaffold sequences can be found, for example, in Jinek, et al. Science (2012) 337(6096):816-821, Ran, et al. Nature Protocols (2013) 8:2281-2308, PCT Publication No. WO2014/093694, and PCT Publication No. WO2013/176772.

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” A gRNA suitable for targeting a target site may comprise a number of domains. In some embodiments, e.g., in some embodiments where a Cas9 nuclease is used, a unimolecular sgRNA, may comprise, from 5′ to 3′:
a targeting domain corresponding to a target site sequence in a target locus;
a first complementarity domain;
a linking domain;
a second complementarity domain (which is complementary to the first complementarity domain);
a proximal domain; and
optionally, a tail domain.

Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

Some exemplary suitable Cas12a gRNA scaffold sequences are provided herein, and additional suitable gRNA scaffold sequences will be apparent to the skilled artisan based on the present disclosure. In some embodiments, e.g., in some embodiments where a Cas12a nuclease is used, a gRNA, may comprise, from 5′ to 3′:
a CRISPR RNA (crRNA) sequence for a CRISPR/Cas nuclease, containing:
   a proximal domain;
   a first complementarity domain;
   a linking domain; and
   a second complementarity domain (which is complementary to the first complementarity domain); and
a targeting domain corresponding to a target site sequence.

Each of these domains is now described in more detail.

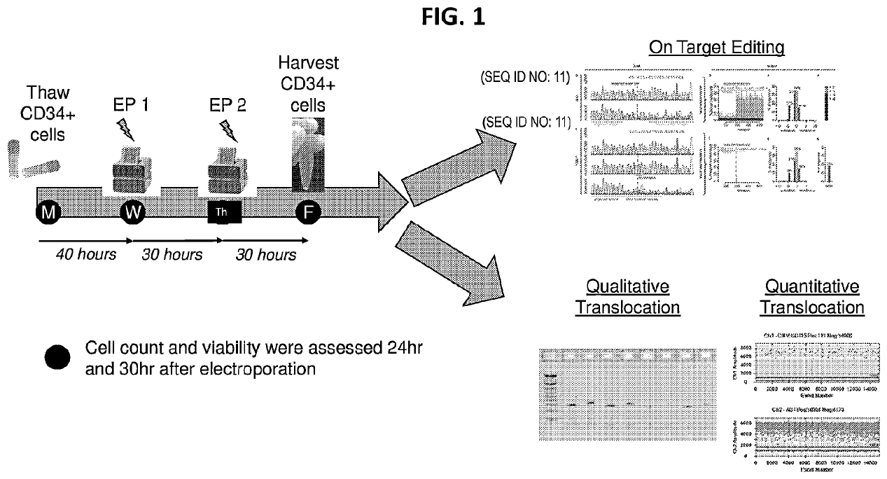

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target sequence. The targeting domain of the gRNA typically comprises an RNA sequence that corresponds to the target sequence in that it resembles the sequence of the targeting domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target sequence, and thus with the strand complementary to the strand that comprises the PAM sequence. It will be understood that the targeting domain of the gRNA typically does not include the PAM sequence. It will further be understood that the location of the PAM may be 5′ or 3′ of the target site sequence, depending on the nuclease employed. For example, the PAM is typically 3′ of the target sequence sequences for Cas9 nucleases, and 5′ of the target sequence for Cas12a nucleases. For an illustration of the location of the PAM and the mechanism of gRNA binding a target site, see, e.g., FIG. 1 of Vanegas et al., Fungal Biol Biotechnol. (2019) 6: 6, which is incorporated by reference herein. For additional illustration and description of the mechanism of gRNA targeting an RNA-guided nuclease to a target site, see Fu Y et al, Nat Biotechnol (2014) (doi: 10.1038/nbt.2808) and Sternberg S H et al., Nature (2014) (doi: 10.1038/nature13011), both incorporated herein by reference.

The targeting domain may comprise a nucleotide sequence that corresponds to the sequence of the target sequence, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

[target domain (DNA)] [PAM]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
| | | | | | | | | | | | | | | | | | | | | |
5′-N-N-N-N-N-N-N-N-N-N-N-N-N-N-N-N-N-N-N-N-N-N-[gRNA scaffold]-3′ (RNA)
[targeting domain (RNA)] [binding domain]

The structure of a typical Cas12a gRNA can be found, for example in FIG. 1 of Zetsche et al. Cell (2015) 163(3): 759-771, which is incorporated by reference herein in its entirety. An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

[PAM] [target domain (DNA)]
5′-T-T-N-N-N-N-N-N-N-N-N-N-N-N-N-N-N-N-N-N-N-N-N-N-N-3′ (DNA)
3′-A-A-N-N-N-N-N-N-N-N-N-N-N-N-N-N-N-N-N-N-N-N-N-N-N-5′ (DNA)
| | | | | | | | | | | | | | | | | | | | | |
5′-[gRNA scaffold]-N-N-N-N-N-N-N-N-N-N-N-N-N-N-N-N-N-N-N-N-N-N-3′ (RNA)
[binding domain] [targeting domain (RNA)]

In some embodiments, the Cas12a PAM sequence is 5′-T-T-T-V-3′. In some embodiments, the Cas12a PAM sequence is 5′-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in the target locus); and
   a first complementarity domain; and
a second strand, comprising, e.g., from 5′ to 3′:
   optionally, a 5′ extension domain;
   a second complementarity domain;
   a proximal domain; and
   optionally, a tail domain.

TABLE 1
Exemplary targeting domain sequences

| Target | Nuclease | Sequence (5′-3′) |
|---|---|---|
| CD19 | SpCas9 | GGAACCTCTAGTGGTGAAGG |
| | | (SEQ ID NO: 1) |
| CD5 | SpCas9 | CATAGCTGATGGTACCCCCC |
| | | (SEQ ID NO: 2) |
| CD33 | SpCas9 | GGTGGGGGCAGCTGACAACC |
| | | (SEQ ID NO: 3) |
| CLL-1 | SpCas9 | TAGCTCACGACATAATTTGG |
| | | (SEQ ID NO: 10) |

In Table 1, “SpCas9” refers to Cas9 nuclease from Streptococcus pyogenes.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a 2′-O-methyl-3′-phosphorothioate nucleotides. In some embodiments, the gRNA may comprise one or more modified nucleotides, e.g., as described in PCT Publication Nos. WO2017/214460, WO2016/089433, and WO2016/164356, which are incorporated by reference their entirety.

The gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNPs into a cell, electroporation of mRNA encoding a CRISPR/Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

In some embodiments, the gRNAs described herein are capable of directing a CRISPR/Cas nuclease to a target site sequence and directing cleavage of one or both strands of DNA at the target site sequence.

### Genetically Engineered Cells and Related Compositions

Aspects of the present disclosure relate to methods for effecting genetic modifications (e.g., mutations) in the genome of a cell in a sequential manner. In some embodiments, the methods described herein produce genetically engineered cells having more than one genetic modification (e.g., 2, 3, 4, 5, or more) resulting from the sequential editing at a first target site, followed by editing at a second target site, and so on. In some embodiments, the methods comprise contacting a cell or population of cells with (i) a first gRNA comprising a targeting domain that binds to a first target sequence and (ii) an RNA-guided nuclease that binds the first gRNA and forms a ribonucleoprotein (RNP) complex that binds to the first target sequence. In some embodiments, binding of the RNP complex to the first target sequence results in a double stranded break of the DNA at or proximal to the first target sequence. In some embodiments, the methods comprise contacting a cell or population of cells with (i) a second gRNA comprising a targeting domain that binds to a second target sequence and (ii) an RNA-guided nuclease that binds the second gRNA and forms a ribonucleoprotein (RNP) complex that binds to the second target sequence, wherein contacting the cell or population of cells with the first gRNA and RNA-guided nuclease and contacting the cell or population of cells with the second gRNA and RNA-guided nuclease are performed sequentially and separated by a time interval. In some embodiments, binding of the RNP complex to the second target sequence results in a double stranded break of the DNA at or proximal to the second target sequence. In some embodiments, the first targeting domain and the second targeting domain are different, e.g., do not have the same nucleotide sequence, do not bind to the same target sequence.

As described herein and as would be evident to one of ordinary skill in the art, generation of a double stranded break (DSB) in the DNA of a cell, for example, by contacting the cell with a gRNA targeting a targeting sequence in the genome of a cell and an RNA-guided nuclease, may be repaired by the cell using any applicable DNA repair mechanism. In general, DSB may be repaired, for example, by non-homologous end-joining (“NHEJ,” also referred to as classical non-homologous end-joining (“c-NHEJ”), microhomology-mediated end-joining (“MMEJ,” also referred to as alternative end-joining (“alt-EJ”)), or homology directed recombination (“HDR”) pathways.

In some embodiments, methods of the disclosure involve introducing a modification to a first target sequence, wherein the modification comprises making a double strand break that is recognized/resolved by a cellular DNA repair mechanism and then introducing a modification to a second target sequence, wherein the modification comprises making a double strand break that is recognized/resolved by a cellular DNA repair mechanism. Without wishing to be bound by theory, the kinetics associated with different cellular DNA repair mechanisms are thought to determine the speed at which a break in genomic DNA is repaired, and thus how long a break persists (e.g., after contacting a cell with a gRNA and RNA-guided nuclease). See, e.g., Chang et al. Nat Rev Mol Cell Biol. (2017) 18(8): 495-506; and Kochan et al. Nucleic Acids Res. (2017) December 15; 45(22): 12625-12637. Without wishing to be bound by theory, it is thought that NHEJ repairs double strand breaks more rapidly than other repair pathways. Furthermore, the DNA repair pathway used to recognize and repair a double stranded break influences the resulting modification (e.g., insertion, deletion, translocation) and size of said modification (e.g., number of nucleotides inserted, deleted). To minimize the time in which a cell of a method disclosed herein comprises multiple breaks in its genomic DNA and therefore reduce/minimize the risk of translocation events, the first target sequence to be modified may be selected such that the DSB is preferentially recognized/repaired by a DNA repair mechanism prior to modification at the second target sequence. In some embodiments, the first gRNA to contact a cell or population of cells may be selected such that the DSB generated with the gRNA and RNA-guided nuclease is preferentially recognized/repaired by a DNA repair mechanism prior to contacting the cell or population of cells with the second gRNA.

In some embodiments, the order of genetic modification is selected based on the predicted rate of DNA repair of a DSB. For example, in some embodiments, a DSB that is predicted to be resolved/repaired at a faster rate is selected as first genetic modification prior to a DSB that is predicated to be resolved/repaired at a slower rate. In some embodiments, contacting the cell or population of cells with the first gRNA and RNA-guided nuclease results in a fast-resolving double strand break. In some embodiments, contacting the cell or population of cells with the second gRNA and RNA-guided nuclease results in a fast-resolving double strand break. In some embodiments, contacting the cell or population of cells with the second gRNA and RNA-guided nuclease results in a slow-resolving double strand break. Without wishing to be bound by theory, it is thought that the nature of a double strand break (e.g., the presence or absence of 3′ and/or 5′ overhangs, overhang length, presence of blunt-ends) influences the speed at which the double strand break is recognized and/or resolved (e.g., generating an insertion or deletion) by cellular DNA repair processes.

As used herein, a fast-resolving double strand break is a double strand break that is detectable for less than 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 36, 37, 38, 39, 40, 41, 42, 43, 44, 45, 46, 47, or 48 hours after contacting a cell or a population of cells with a break generating agent (e.g., a gRNA and RNA-guided nuclease). In some embodiments, a fast-resolving double strand break is detectable in a cell or population of cells for less than 14, 12, 10, 8, 6, 4, 2, or 1 hour after contacting a cell or a population of cells with a break generating agent (e.g., a gRNA and RNA-guided nuclease). In some embodiments, a fast-resolving double strand break is detectable in a cell or population of cells for less than 120, 110, 100, 90, 80, 70, 60, 50, 40, 30, 20, 10, 5, or 1 minute after contacting a cell or a population of cells with a break generating agent (e.g., a gRNA and RNA-guided nuclease).

As used herein a slow-resolving double strand break is a double strand break that is detectable for at least 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours after contacting the cell or population of cells with a break generating agent (e.g., a gRNA and RNA-guided nuclease). In some embodiments, a slow-resolving double strand break is detectable in a cell or population of cells for at least 24, 28, 32, 36, or 40 hours after contacting a cell or a population of cells with a break generating agent (e.g., a gRNA and RNA-guided nuclease). Without wishing to be bound by theory, by selecting a first target sequence and/or first gRNA to make the modification to the first target sequence comprise a fast-resolving double strand break, the overlap time in which the cell comprises two double stranded breaks in the genome (i.e., a first fast-resolving double strand break and a second double strand break) associated with a modification to a second target sequence is minimized or eliminated, thereby decreasing the level of or eliminating translocation products, or risk of translocation products.

In some embodiments, the first target sequence to be modified may be selected such that the DSB is preferentially recognized/repaired by the NHEJ repair mechanism. In some embodiments, the first gRNA to contact a cell or population of cells may be selected such that the DSB generated with the gRNA and RNA-guided nuclease is preferentially recognized/repaired by the NHEJ repair mechanism.

In some embodiments, the second target sequence to be modified may be selected such that the DSB is preferentially recognized/repaired by the NHEJ or a non-NHEJ repair mechanism (e.g., homologous recombination or MMEJ). In some embodiments, the second target sequence to be modified may be selected such that the DSB is preferentially recognized/repaired by the MMEJ repair mechanism. In some embodiments, the second gRNA to contact a cell or population of cells may be selected such that the DSB generated with the gRNA and RNA-guided nuclease is preferentially recognized/repaired by the NHEJ or a non-NHEJ repair mechanism (e.g., homologous recombination or MMEJ). In some embodiments, the second gRNA to contact a cell or population of cells may be selected such that the DSB generated with the gRNA and RNA-guided nuclease is preferentially recognized/repaired by the MMEJ repair mechanism.

In some embodiments, contacting a cell or population of cells with a first gRNA and RNA-guided nuclease and contacting the cell or population of cells with a second gRNA and RNA-guided nuclease are separated by a time interval. The time interval may be selected based on factors such as ensure a break in the DNA associated with the modification to the first target domain is substantially (e.g., completely) repaired (e.g., generating an insertion or deletion) prior to formation of a different break in the DNA associated with the modification to the second target domain. In some embodiments, the time interval is sufficient such that at least 5%, 10%, 15%, 20%, 25%, 30%, 35%, 40%, 45%, 50%, 55%, 60%, 65%, 70%, 75%, 80%, 85%, 90%, 95%, or 100% of the DSB are repaired before contacting the cell or population of cells with the second gRNA and RNA-guided nuclease.

In some embodiments, the time interval between the first double strand break-generating step (e.g., between contacting a cell with a first gRNA and an RNA-guided nuclease) and the second double strand break-generating step (e.g., contacting the cell with a second gRNA and an RNA-guided nuclease) is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 hours (and optionally no more than 100, 90, 80, 70, 60, 50, 40, 30, or 20 hours). In some embodiments, the time interval between first double strand break-generating step (e.g., between contacting a cell with a first gRNA and an RNA-guided nuclease) and the second double strand break-generating step (e.g., contacting the cell with a second gRNA and an RNA-guided nuclease) is 10-100, 12-100, 15-100, 18-100, 20-100, 24-100, 28-100, 30-100, 36-100, 42-100, 48-100, 54-100, 60-100, 70-100, 80-100, 90-100, 10-80, 12-80, 15-80, 18-80, 20-80, 24-80, 28-80, 30-80, 36-80, 42-80, 48-80, 54-80, 60-80, 70-80, 10-60, 12-60, 15-60, 18-60, 20-60, 24-60, 28-60, 30-60, 36-60, 42-60, 48-60, 54-60, 10-54, 12-54, 15-54, 18-54, 20-54, 24-54, 28-54, 30-54, 36-54, 42-54, 48-54, 10-48, 12-48, 15-48, 18-48, 20-48, 24-48, 28-48, 30-48, 36-48, 42-48, 10-42, 12-42, 15-42, 18-42, 20-42, 24-42, 28-42, 30-42, 36-42, 10-36, 12-36, 15-36, 18-36, 20-36, 24-36, 28-36, 30-36, 10-30, 12-30, 15-30, 18-30, 20-30, 24-30, 28-30, 10-28, 12-28, 15-28, 18-28, 20-28, 24-28, 10-24, 12-24, 15-24, 18-24, 20-24, 10-20, 12-20, 15-20, 18-20, 10-18, 12-18, 15-18, 10-15, 12-15, or 10-12 hours. In some embodiments, the time interval between first double strand break-generating step (e.g., between contacting a cell with a first gRNA and an RNA-guided nuclease) and the second double strand break-generating step (e.g., contacting the cell with a second gRNA and an RNA-guided nuclease) is about 30 hours.

As described herein, the present disclosure is based, in part, on the discovery that the order and/or timing of sequential genetic modifications are made may contribute to the level of undesired translocation products produced in the cell or population of cells. Without wishing to be bound by theory, while it is desirable to produce genetically engineered cells comprising multiple genomic DNA modifications, it is thought that the presence of a plurality of breaks (e.g., double strand breaks) in the genomic DNA of a cell at substantially the same time should be decreased or avoided to reduce the likelihood of cellular DNA repair mechanisms repairing the breaks in a manner that produces a translocation product.

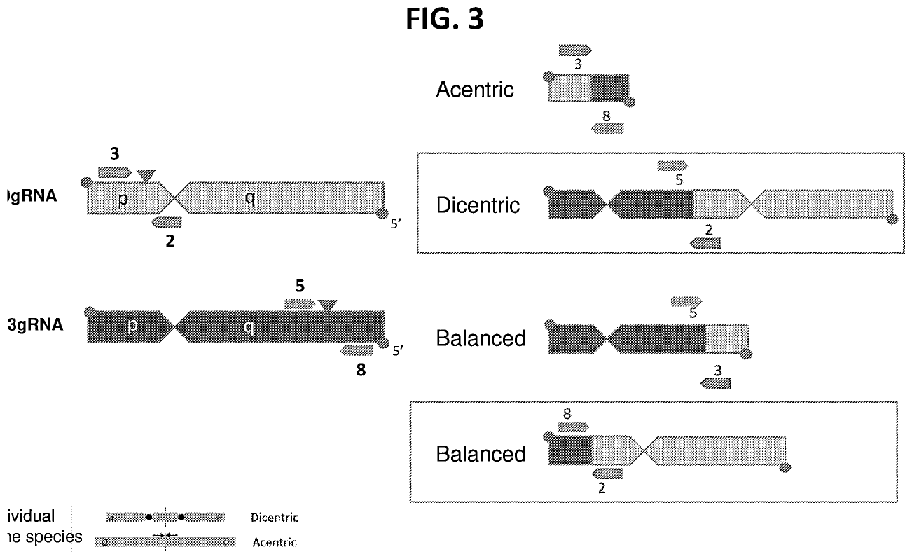

The term “translocation product” is used herein to refer to a nucleic acid comprising at least two portions of genomic DNA that do not naturally occur contiguous to one another. For example, a portion of a first chromosome and a portion of second chromosome may be joined resulting in a fusion of the first and second chromosomes (e.g., chromosomal rearrangements). Alternatively, a first portion of a chromosome and a second portion of the same chromosome may be joined in an orientation that is not naturally occurring, such as an inversion. See, e.g., Modern Genetic Analysis. “Chromosomal Rearrangements” Griffiths A J F, Gelbart W M, Miller J H, et al. New York: W. H. Freeman; 1999. In some embodiments, a translocation product is formed by cellular DNA repair mechanisms repairing a plurality of breaks to the genomic DNA. FIG. 3 shows a number of exemplary translocation products, including acentric, dicentric, and balanced products. In some embodiments, a translocation product comprises most or all of a naturally occurring chromosome or two naturally occurring chromosomes. In some embodiments, a translocation product comprises less than 50, 40, 30, 20, or 10% of a naturally occurring chromosome. In some embodiments, a translocation product comprises a single centromere. In some embodiments, a translocation product comprises more than one centromere, e.g., two centromeres (i.e., the translocation product is dicentric). In some embodiments, a translocation product comprises no centromere (i.e., the translocation product is acentric). In some embodiments, the translocation products are balanced, meaning that no genetic information is removed or duplicated. Examples of balanced translocation products include reciprocal translocations and inversions. In reciprocal translocations, two acentric fragments of two chromosomes trade places (see, FIG. 3, “balanced” schematics). In inversion translocations, more than two fragments of a chromosome have been generated and the fragments are arranged in an inverted orientation. In some embodiments, the translocation products are imbalanced, such as deletions (loss of genetic information) and duplications (duplication of genetic information).

The term “translocation product cell” is used herein to refer to a cell comprising one or more translocation products. The presence of a translocation product, as well as the type of translocation product (e.g., acentric, dicentric, balanced), may be assessed by methods known in the art, for example by DNA sequencing, polymerase chain reaction (PCR) amplification of a product.

The methods of the disclosure may employ one or more means to decrease or eliminate the formation of translocation products and translocation product cells comprising said translocation products. For example, the methods described herein involve introducing a modification to a first target domain in a first step and a modification to a second target domain in a second step, separating the two steps by a time interval (e.g., that is selected to decrease or eliminate the overlapping occurrence of double strand breaks). As a further example, the methods described herein may involve introducing a modification to a first target domain, wherein the modification comprises making a double strand break that is recognized/resolved by NHEJ, and then introduce a modification to a second target domain, wherein the modification comprises making a double strand break that is recognized/resolved by any cellular DNA repair mechanism (e.g., NHEJ or a non-NHEJ pathway, e.g., homologous recombination or MMEJ). As an additional example, a method of the disclosure may introduce a modification to a first target domain, wherein the modification comprises making a fast-resolving double strand break, and then introduce a modification to a second target domain, wherein the modification comprises making a fast- or slow-resolving double strand break (e.g., a slow-resolving double strand break).

In some embodiments, a method of the disclosure comprises introducing a modification to a first target domain comprising a sequence encoding a first lineage-specific cell-surface antigen, and then introducing a modification to second target domain comprising a sequence encoding a lineage-specific cell-surface antigen. In some embodiments, the first lineage-specific cell-surface antigen is CD33. In some embodiments, the second lineage-specific cell-surface antigen is CD19, CLL-1, or CD5.

In some embodiments, the methods described herein produce a subpopulation of translocation product cells. In some embodiments, each translocation product cell comprises at least one translocation product. In some embodiments, the translocation product comprises a nucleic acid (e.g., a portion of the genome) comprising the first target domain or a portion thereof and the second target domain or a portion thereof. The translocation product may be formed by cellular DNA repair of a double strand break in or proximal to the first target domain and a double strand break in or proximal to the second target domain in a manner that connects the first target domain or portion thereof to the second target domain or a portion thereof. See, e.g., FIG. 3.

In some embodiments, the methods described herein produce at least 1, 3, 5, 10, 15, 20, 25, 30, 40, 50, 60, 70, 80, 90, 91, 92, 93, 94, 95, 96, 97, 98, 99 or 100% fewer translocation product cells as compared to the number (or percentage) of translocation product cells produced using methods that introduce a modification to the second target sequence (e.g., contacting the cell with the second gRNA) prior to introducing a modification to the first target sequence (e.g., contacting the cell with the first gRNA). In some embodiments, the method disclosed herein produces 1-10%, 1-20%, 1-30%, 1-40%, 1-50%, 1-60%, 1-70%, 1-80%, 1-90%, 1-100%, 10-20%, 10-30%, 10-40%, 10-50%, 10-60%, 10-70%, 10-80%, 10-90%, 10-100%, 20-30%, 20-40%, 20-50%, 20-60%, 20-70%, 20-80%, 20-90%, 20-100%, 30-40%, 30-50%, 30-60%, 30-70%, 30-80%, 30-90%, 30-100%, 40-50%, 40-60%, 40-70%, 40-80%, 40-90%, 40-100%, 50-60%, 50-70%, 50-80%, 50-90%, 50-100%, 60-70%, 60-80%, 60-90%, 70-100%, 70-80%, 70-90%, 70-100%, 80-90%, 80-100%, or 90-100% fewer translocation product cells as compared to the number (or percentage) of translocation product cells produced using methods that introduce a modification to the second target sequence (e.g., contacting the cell with the second gRNA) prior to introducing a modification to the first target sequence (e.g., contacting the cell with the first gRNA).

In some embodiments, the methods described herein produce at least 1, 3, 5, 10, 15, 20, 25, 30, 40, 50, 60, 70, 80, 90, 91, 92, 93, 94, 95, 96, 97, 98, 99 or 100% fewer translocation product cells as compared to the number (or percentage) of translocation product cells produced using methods involving introducing a modification to the first target sequence and the second target sequence at substantially the same time (e.g., simultaneously), e.g., contacting the cell with the first gRNA and the second gRNA at substantially the same time. In some embodiments, the method disclosed herein produces 1-10%, 1-20%, 1-30%, 1-40%, 1-50%, 1-60%, 1-70%, 1-80%, 1-90%, 1-100%, 10-20%, 10-30%, 10-40%, 10-50%, 10-60%, 10-70%, 10-80%, 10-90%, 10-100%, 20-30%, 20-40%, 20-50%, 20-60%, 20-70%, 20-80%, 20-90%, 20-100%, 30-40%, 30-50%, 30-60%, 30-70%, 30-80%, 30-90%, 30-100%, 40-50%, 40-60%, 40-70%, 40-80%, 40-90%, 40-100%, 50-60%, 50-70%, 50-80%, 50-90%, 50-100%, 60-70%, 60-80%, 60-90%, 70-100%, 70-80%, 70-90%, 70-100%, 80-90%, 80-100%, or 90-100% fewer translocation product cells as compared to the number (or percentage) of translocation product cells produced using methods that introduce a modification to the first target sequence and the second target sequence at substantially the same time (e.g., simultaneously), e.g., contacting the cell with the first gRNA and the second gRNA at substantially the same time.

Genetically Engineering Cells and Compositions Comprising or Associated with Said Cells

In some aspects, the present disclosure provides methods of effectively generating multiple (e.g., at least 2, 3, 4, 5, or more) genetic modifications (e.g., mutations) in the genome of a cell, in a manner that reduces the translocation events (translocation products) or the risk of translocation events. In some aspects, the disclosure is directed to cells and cell populations comprising a genetically engineered cell or plurality of genetically engineered cells, wherein the genetically engineered cell comprises a first genomic modification and a second genomic modification, wherein the first target domain is different from the second target domain, and wherein the first genomic modification was made prior to the second genomic modification. In some embodiments, the first genomic modification consists of an insertion or deletion within or immediately proximal to a first target domain in the genome of the genetically engineered cell. In some embodiments, the first genomic modification is an insertion or deletion generated by NHEJ (e.g., NHEJ repair of a double strand break). In some embodiments, the second genomic modification consists of an insertion or deletion within or immediately proximal to a second target domain in the genome of the genetically engineered cell. In some embodiments, the second genomic modification is an insertion or deletion generated by NHEJ or a non-NHEJ repair process (e.g., Microhomology-Mediated End Joining (MMEJ) or homologous recombination) (e.g., NHEJ or non-NHEJ repair of a double strand break).

In some embodiments, the first genomic modification consists of an insertion or deletion within or immediately proximal to a first target domain in the genome of the genetically engineered cell, wherein the insertion or deletion was produced by a fast-resolving double strand break (e.g., repair of a fast-resolving double strand break). In some embodiments, the second genomic modification consists of an insertion or deletion within or immediately proximal to a second target domain in the genome of the genetically engineered cell, wherein the insertion or deletion was produced by a fast-revolving double strand break or a slow-resolving double strand break (e.g., repair of a fast-resolving double strand break or a slow-resolving double strand break).

In some embodiments, a cell produced using the methods described herein comprises fewer translocation products than an otherwise similar cell in which the first genomic modification was made after the second genomic modification. In some embodiments, a cell population produced using the methods described herein comprises fewer translocation products cells than an otherwise similar cell population in which the first genomic modification was made after the second genomic modification.

In some embodiments, a cell produced using the methods described herein comprises fewer translocation products than an otherwise similar cell in which the first genomic modification and the second genomic modification were made at substantially the same time (e.g., simultaneously). In some embodiments, a cell produced using the methods described herein comprises fewer translocation products than an otherwise similar cell in which the cell was contacted with a first gRNA comprising a first targeting domain at substantially the same time (e.g., simultaneously) at the cell was contacted with a second gRNA comprising a second targeting domain.

In some embodiments, a cell population produced using the methods described herein comprises fewer translocation product cells than an otherwise similar cell population in which the first genomic modification and the second genomic modification were made at substantially the same time (e.g., simultaneously). In some embodiments, a cell population produced using the methods described herein comprises fewer translocation products than an otherwise similar cell population in which the cell population was contacted with a first gRNA comprising a first targeting domain at substantially the same time (e.g., simultaneously) at the cell population was contacted with a second gRNA comprising a second targeting domain. In some embodiments, less than 90, 80, 70, 60, 50, 40, 30, 20, 10, 9, 8, 7, 6, 5, 4, 3, 2, 1, 0.5, 0.25, or 0.1% (e.g., 0%) of the cells of the cell population are translocation product cells.

Accordingly, provided herein are genetically engineered cells, populations thereof, and cells descended therefrom, that are produced using methods described herein (e.g., using oligonucleotides described herein). Also provided are pharmaceutical compositions comprising said cell(s), e.g., and one or more pharmaceutically acceptable carriers and/or excipients.

The methods described herein may be applied to any cell or cell type capable of being genetically engineered using a CRISPR/Cas system as described herein. In some embodiments, the cell is a eukaryotic cell. In some embodiments, the cell is a mammalian cell, yeast cell, fungal cell, or plant cell. In some embodiments, the cell is a human cell or a mouse cell. In some embodiments, the cells may be obtained from a subject, such as a human subject. In some embodiments, the cells are obtained from a human subject, such as a human subject having a disease or disorder, such as a hematopoietic malignancy. In some embodiments, the cells are obtained from a healthy donor. Methods of obtaining mammalian cells, such as hematopoietic stem cells, are described, e.g., in PCT/US2016/057339, which is herein incorporated by reference in its entirety. In some embodiments, the mammalian subject is a non-human primate, a rodent (e.g., mouse or rat), a bovine, a porcine, an equine, or a domestic animal.

In some embodiments, the HSCs are obtained from the subject to whom the immune cells expressing the chimeric receptors will be subsequently administered. Cells that are administered to the same subject from which the cells were obtained are referred to as autologous cells, whereas cells that are obtained from a subject who is not the subject to whom the cells will be administered are referred to as allogeneic cells.

In some embodiments, the cells provided herein are stem cells. In some embodiments, the stem cells are embryonic stem cells, adult stem cells, induced pluripotent stem cells, cord blood stem cells, or amniotic fluid stem cells. In some embodiments, the stem cells are hematopoietic stem cells, mesenchymal stem cells, neural stem cells, epithelial stem cells, or skin stem cells. In some embodiments, the cells provided herein are progenitor cells, which are cells descended from a stem cell and capable to differentiate into a plurality of cell types.

In some embodiments, the cells provided herein are hematopoietic cells, e.g., hematopoietic stem cells (HSC) or hematopoietic progenitor cells (HPC). In some embodiments, the cells provided herein hematopoietic stem or progenitor cells. Hematopoietic stem cells (HSCs) are typically capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage. In some embodiments, the HSCs are peripheral blood HSCs.

In some embodiments, the cells provided herein are immune effector cells. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell.

The skilled artisan will understand, however, that the provision of such examples is for the purpose of illustrating some specific embodiments, and additional suitable cells and cell types will be apparent to the skilled artisan based on the present disclosure, which is not limited in this respect.

In some embodiments, a genetically engineered cell provided herein comprises more than one genomic modification, e.g., more than one genomic modification that results in a reduced or loss of expression of a protein, for example a protein encoded by or regulated by the target site sequence, or expression of a variant form of the proteins. It will be understood that the gene editing methods provided herein may result in genomic modifications in one or both alleles of a target genetic loci. In some embodiments, genetically engineered cells comprising a genomic modification in both alleles of a given genetic locus are preferred.

In some embodiments, genetic modifications effecting both alleles of a target genetic loci are referred to herein as a “biallelic” modification. In some embodiments, gene editing approaches on the present invention result in biallelic deletion of a target genetic loci. Examples of target genetic loci that may undergo editing procedures resulting in biallelic deletion include CD33 and CLL-1 In some embodiments, biallelic deletion is characterized by genetic analyses reflecting what percentage of cells in a given population comprise biallelic deletion of CD33 and/or CLL-1. In some embodiments, the genetic analyses use to detect or characterize biallelic deletion include indel analysis using TIDE analysis of NGS data. In some embodiments, methods and compositions of the present invention result in biallelic deletion of CD33 and/or CLL-1 in more than 80% of the cells in a population that was electroporated with RNPs directed toward CD33 and/or CLL-1 as the target genetic loci. In some embodiments, more than 80% of cells in a population comprising biallelic deletion of CD33 and/or CLL-1 means that the desired editing outcomes are present in about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the cells in the population.

In some embodiments, a genetically engineered cell provided herein comprises two or more genomic modifications. For example, a population of genetically engineered cells can comprise a plurality of different mutations, such as two or more mutations in the same or different genetic loci in a cell.

As will be evident to one of ordinary skill in the art, the compositions and methods described herein may be used to modify any genetic locus in a cell, including for example protein-coding, non-protein coding, chromosomal, and extra-chromosomal sequences. Accordingly, targeting domains of gRNAs may be designed to target any genetic locus (i.e., a target site sequence), such as a target site sequence adjacent to a PAM sequence for a corresponding CRISPR/Cas nuclease.

In some embodiments, the targeting domain of a gRNA (e.g., the first gRNA, the second gRNA) targets a cell surface protein, such as a Type 0, Type 1, or Type 2 cell surface protein. See, e.g., PCT Publication No. WO 2017/066760. In some embodiments, the targeting domain targets BCMA, CD19, CD20, CD30, ROR1, B7H6, B7H3, CD23, CD33, CD38, C-type lectin like molecule-1 (CLL-1, also referred to herein as CLL1), CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and/or CD26.

In some embodiments, the targeting domain of a gRNA (e.g., the first gRNA, the second gRNA) targets a cell surface protein associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

Additional non-limiting examples of cell-surface proteins include CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213al, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

Compositions and methods (e.g., exemplary gRNAs) for genetic editing and/or inhibition of genes encoding cell surface proteins (e.g., lineage specific antigens) are known to those of skill in the art and include, but are not limited to, those taught in PCT publications WO2017/066760, W02020/047164A1, W02020/150478A1, W02020/237217A1, W02021/041971A1, and W02021/041977A1, which are incorporated by reference in their entirety. Additional compositions and methods (e.g., exemplary gRNAs) for genetic editing and/or inhibition of genes are known to those of skill in the art and include, but are not limited to, those taught in PCT publications W02017/186718A1 and W02018/083071A1, and in Mandal et al. Cell Stem Cell. (2014) 15(5): 643-52, which are incorporated by reference in their entirety.

In some embodiments, the first gRNA comprises a targeting domain that binds to a target sequence in CD33. In some embodiments, the first target sequence is within or associated with the gene encoding CD33. In some embodiments, the second gRNA targets a second lineage-specific cell-surface antigen, such as a lineage-specific cell-surface antigen selected from: BCMA, CD19, CD20, CD30, ROR1, B7H6, B7H3, CD23, CD33, CD38, C-type lectin like molecule-1 (CLL-1), CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and/or CD26. In some embodiments, the first gRNA binds to a target sequence in CD33 and the second gRNA binds to a target sequence in CD19. In some embodiments, the first gRNA binds to a target sequence in CD33 and the second gRNA binds to a target sequence in CD5. In some embodiments, the first gRNA binds to a target sequence in CD33 and the second gRNA binds to a target sequence in CLL-1. In some embodiments, the first gRNA binds to a target sequence in CLL-1 and the second gRNA binds to a target sequence in CD33.

In some embodiments, the first gRNA comprises a targeting domain that binds to a target sequence in CD5. In some embodiments, the first target sequence is within or associated with the gene encoding CD5. In some embodiments, the second gRNA targets a second lineage-specific cell-surface antigen, such as a lineage-specific cell-surface antigen selected from: BCMA, CD19, CD20, CD30, ROR1, B7H6, B7H3, CD23, CD33, CD38, C-type lectin like molecule-1 (CLL-1), CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and/or CD26. In some embodiments, the first gRNA binds to a target sequence in CD5 and the second gRNA binds to a target sequence in CD33.

A method of the disclosure may comprise contacting a cell with n different gRNAs, where n is an integer ≥2, wherein each of the n different gRNAs comprise a targeting domain complementary to a target sequence. In some embodiments, n is at least 2, 3, 4, 5, 6, 7, 8, 9, 10, 11, 12, 13, 14, 15, 16, 17, 18, 19, or 20 (and optionally, no more than 30, 25, 20, 15, or 10). In some embodiments, each of the n different gRNAs comprises a targeting domain complementary to a different target sequence, each separated by time interval (e.g., a time interval sufficient such that the prior DNA break is resolved/repaired or substantially resolved/repaired, thereby minimizing the risk of translocation events.

In some embodiments, a mutation effected by the methods provided herein, e.g., a mutation in a target gene results in a loss of function of a gene product encoded by the target gene. In some embodiments, the loss of function is a reduction in the level of expression of the gene product, e.g., reduction to a lower level of expression, or a complete abolishment of expression of the gene product. In some embodiments, the mutation results in the expression of a variant of the gene product, such as a non-functional variant or a variant having a different function as compared to the wild-type counterpart. For example, in the case of the mutation generating a premature stop codon in the encoding sequence, a truncated gene product, or, in the case of the mutation generating a nonsense or missense mutation, a gene product characterized by an altered amino acid sequence, which renders the gene product non-functional. In some embodiments, the function of a gene product is binding or recognition of a binding partner. In some embodiments, the reduction in expression of the first protein, second protein, or both, is to less than or equal to 50%, less than or equal to 40%, less than or equal to 30%, less than or equal to 20%, less than or equal to 10%, less than or equal to 5%, less than or equal to 2%, or less than or equal to 1% of the level in a wild-type or non-engineered counterpart cell.

In some embodiments, the expression of a protein encoded by a first gene comprising the first target sequence, a protein encoded by a second gene comprising the second target sequence, or both on the genetically engineered cell (e.g., a genetically engineered hematopoietic cell) is compared to the expression of the corresponding protein or both on a naturally occurring hematopoietic cell (e.g., a wild-type counterpart).

In some embodiments, the expression of a first protein, a second protein, or both in the genetically engineered cell (e.g., a genetically engineered hematopoietic cell) is compared to the expression of the first protein, the second proteins, or both in a naturally occurring cell (e.g., a wild-type counterpart hematopoietic cell). In some embodiments, the genetic engineering results in a reduction in the expression level of the protein, the second protein, or both by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the first protein, the second protein, or both on a naturally occurring cell (e.g., a wild-type counterpart hematopoietic cell). For example, in some embodiments, the genetically engineered cell (e.g., a genetically engineered hematopoietic cell) expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the first protein, the second protein, or both as compared to a naturally occurring cell (e.g., a wild-type counterpart hematopoietic cell).

In some embodiments, the expression of a first lineage-specific cell-surface antigen, a second lineage-specific cell-surface antigen, or both on the genetically engineered cell (e.g., a genetically engineered hematopoietic cell) is compared to the expression of the first lineage-specific cell-surface antigen, the second lineage-specific cell-surface antigen, or both on a naturally occurring cell (e.g., a wild-type counterpart hematopoietic cell). In some embodiments, the genetic engineering results in a reduction in the expression level of the first lineage-specific cell-surface antigen, the second lineage-specific cell-surface antigen, or both by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the first lineage-specific cell-surface antigen, the second lineage-specific cell-surface antigen, or both on a naturally occurring cell (e.g., a wild-type counterpart hematopoietic cell). For example, in some embodiments, the genetically engineered cell (e.g., a genetically engineered hematopoietic cell) expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the first lineage-specific cell-surface antigen, the second lineage-specific cell-surface antigen, or both as compared to a naturally occurring cell (e.g., a wild-type counterpart hematopoietic cell).

### Methods of Administration to Subjects in Need Thereof

Some aspects of this disclosure provide methods comprising administering to a subject in need thereof a composition described herein, e.g., a cell genetically engineered via the methods described herein, a population of cells or descendants thereof, or a pharmaceutical composition comprising the same. The cell, population of cells, or descendants thereof may comprise one or more modifications (e.g., genetic modifications) relative to a wildtype cell. In some embodiments, the cell, population of cells, or descendants thereof comprise a modification to a first gene relative to a wildtype cell of the same type. In some embodiments, the cell, population of cells, or descendants thereof comprise a modification to a second gene relative to a wildtype cell of the same type. Genes modified may correspond to any genetic locus targetable by the methods described herein, e.g., a gene encoding a cell-surface protein described herein.

In some embodiments, the methods further involve administering to the subject a therapeutically effective amount of at least one agent that targets a product encoded by a wildtype copy of the modified gene. Without wishing to be bound by theory, by administering an agent that targets a product encoded by a wildtype copy of the modified gene in combination with a cell, population of cells, or descendants thereof comprising the modified gene, it is possible to target cells within a subject with the agent (e.g., disease cells, e.g., cancer cells) while not targeting or targeting to a lesser degree the cell, population of cells, or descendants thereof. For example, such a method may be used to selectively ablate or kill a target cell population in a subject while in combination replenishing the subject with new cells not vulnerable to the agent. As a further example, such a method may administer the agent as a part of the cell, population of cells, or descendants thereof (e.g., a CAR-T therapeutic), and would thus avoid or decrease cell fratricide. In some embodiments, administration of the at least one agent targeting the product encoded by the wildtype copy of the modified gene occurs simultaneously or in temporal proximity with administration of the cell, population or descendant thereof, or the pharmaceutical composition. In some embodiments, administration of the at least one agent targeting the product encoded by the wildtype copy of the modified gene occurs after administration of the cell, population or descendant thereof, or the pharmaceutical composition. In some embodiments, administration of the at least one agent targeting the product encoded by the wildtype copy of the modified gene occurs before administration of the cell, population or descendant thereof, or the pharmaceutical composition. In some embodiments, where the cell, population of cells, or descendants thereof comprises a modification to a first gene and a second gene relative to a wildtype cell of the same type, the method may comprise administering one or more (e.g., two agents) targeting the products of the first gene and the second gene (e.g., wildtype copies of the first gene and the second gene).

A subject in need thereof is, in some embodiments, a subject undergoing or about to undergo an immunotherapy targeting a product of the first gene and/or second gene. A subject in need thereof is, in some embodiments, a subject having or having been diagnosed with, a malignancy, such as caner (e.g., cancer associated with the presence of cancer stem cells, a hematopoietic malignancy, a cancer characterized by expression of a product of the first and/or second gene. In some embodiments, a subject having such a malignancy may be a candidate for administration of the agent, such as an immunotherapeutic, targeting a product of the first gene and/or second gene, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by the agent.

In some embodiments, the malignancy is a hematologic malignancy, or a cancer of the blood. In some embodiments, the malignancy is a lymphoid malignancy or a myeloid malignancy.

In some embodiments, the malignancy is an autoimmune disease or disorder. Examples of autoimmune disorders include, without limitation, rheumatoid arthritis, multiple sclerosis, leukemia, graft-versus host disease, lupus, and psoriasis.

In some embodiments, the malignancy is graft-versus host disease.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting a product of the first gene and/or second gene, e.g., CAR-T cell therapy, wherein the immune effector cells express a CAR targeting the product, and wherein at least a subset of the immune effector cells also express the product on their cell surface. As used herein, the term “fratricide” refers to self-killing. For example, cells of a population of cells kill or induce killing of cells of the same population. In some 5 embodiments, cells of the immune effector cell therapy kill or induce killing of other cells of the immune effector cell therapy.

In such embodiments, fratricide ablates a portion of or the entire population of immune effector cells before a desired clinical outcome, e.g., ablation of malignant cells expressing the product within the subject, can be achieved. In some such embodiments, using genetically engineered immune effector cells, as provided herein, e.g., immune effector cells that do not express the product or do not express a variant of the product recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express the product or do not express a variant of the product recognized by the CAR, may be further modified to also express the agent (e.g., a CAR targeting the product). In some embodiments, the immune effector cells may be lymphocytes, e.g., T-lymphocytes, such as, for example alpha/beta T lymphocytes, gamma/delta T-lymphocytes, or natural killer T cells. In some embodiments, the immune effect or cells may be natural killer (NK) cells.

In some embodiments, an effective number of genetically engineered cells as described herein, comprising modifications in their genome is administered to a subject in need thereof, e.g., a subject undergoing or that will undergo a therapy targeting a product of the first gene and/or second gene, wherein the therapy is associated or is at risk of being associated with a detrimental on-target, off-disease effect, e.g., in the form of cytotoxicity towards healthy cells in the subject that express the product. In some embodiments, an effective number of such genetically engineered cells may be administered to the subject in combination with the agent targeting a product encoded by a first gene or a second gene.

It is understood that when genetically modified cells and agents targeting a product encoded by a first gene or a second gene (e.g., an immunotherapeutic agent) are administered in combination, the cells and the agent may be administered at the same time or at different times, e.g., in temporal proximity.

For example, in some embodiments, administration in combination includes administration in the same course of treatment, e.g., in the course of treating a subject with an agent targeting a product (e.g., immunotherapy), the subject may be administered an effective number of genetically engineered cells, simultaneously, concurrently, or sequentially, e.g., before, during, or after the treatment with the agent, and/or in any order with respect to each other and the cells, population of cells, or descendants thereof. Furthermore, the cells and the agent may be admixed or in separate volumes or dosage forms.

In some embodiments, the agent that targets a product encoded by the first gene or a wildtype copy thereof is an immunotherapeutic agent. In some embodiments, the agent that targets a product encoded by the first gene or a wild-type copy thereof comprises an antigen binding fragment that binds the product encoded by the first gene or a wildtype copy thereof. In some embodiments, the agent that targets a product encoded by the first gene or a wild-type copy thereof comprises an antigen binding fragment that binds the product encoded by the second gene or a wildtype copy thereof.

In some embodiments, the agent is an immune cell that expresses a chimeric antigen receptor, which comprises an antigen-binding fragment (e.g., a single-chain antibody) capable of binding to a product produced by the first gene or a wild-type copy thereof. In some embodiments, the agent is an immune cell that expresses a chimeric antigen receptor, which comprises an antigen-binding fragment (e.g., a single-chain antibody) capable of binding to a product produced by the second gene or a wild-type copy thereof. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise an antibody fragment that binds a product encoded by the first gene or a wildtype copy thereof, a product encoded by the second gene or a wildtype copy thereof, or both. The antibody fragment can comprise one or more CDRs, the variable regions (or portions thereof), the constant regions (or portions thereof), or combinations of any of the foregoing.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table 2 below.

TABLE 2
Exemplary components of a chimeric receptor

| Chimeric receptor | |
|---|---|
| component | Amino acid sequence |
| Antigen-binding | Light chain- |
| fragment | Linker-Heavy chain |
| CD28 | IEVMYPPPYLDNEKSNGTIIHVKGKHLCP |
| costimulatory | SPLFPGPSKPFWVLVVVGGVLACYSLLVTV |
| domain | AFIIFWVRSKRSRLLHSDYMNMTPRRPGPT |
| | RKHYQPYAPPRDFAAYRS |
| | (SEQ ID NO: 4) |
| CD8alpha | IYIWAPLAGTCGVLLLSLVITLYC |
| transmembrane | (SEQ ID NO: 5) |
| domain | |
| CD28 | FWVLVVVGGVLACYSLLVTVAFII |
| transmembrane | FWVRSKRSRLLHSDYMNMTPRR |
| domain | PGPTRKHYQPYAPPRDFAAYRS |
| | (SEQ ID NO: 6) |
| 4-1BB | KRGRKKLLYIFKQPFMRVQTTQEEDGCS |
| intracellular | CRFPEEEEGGCEL |
| domain | (SEQ ID NO: 7) |
| CD35ζ | RVKFSRSADAPAYQQGQNQLYNELNLG |
| cytoplasmic | RREEYDVLDKRRGRDPEMGGKPQRRKNP |
| signaling | QEGLYNELQKDKMAEAYSEIGMKGERRR |
| domain | GKGHDGLYQGLSTATKDTYDALHMQALPPR |
| | (SEQ ID NO: 8) |

In some embodiments, the number of genetically engineered cells provided herein, e.g., HSCs, HPCs, or immune effector cells (e.g., CAR-expressing cells) that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells (e.g., CAR-expressing cells) that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells (e.g., CAR-expressing cells) that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the agent that targets a product encoded by the first gene or a wildtype copy thereof is an antibody-drug conjugate (ADC). The ADC may be a molecule comprising an antibody or antigen-binding fragment 5 thereof conjugated to a toxin or drug molecule. Binding of the antibody or fragment thereof to the corresponding antigen allows for delivery of the toxin or drug molecule to a cell that presents the antigen on the cell surface (e.g., target cell), thereby resulting in death of the target cell.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BJIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-10⁷, MEDI4276, DSTA4637S/RG7861. Anti-CD30 antibody drug conjugates are known in the art, for example, Bradley et al. Am. J. Health Syst. Pharm. (2013) 70(7): 589-97; Shen et al. mAbs (2019) 11(6): 1149-1161.

In some embodiments, binding of the antibody-drug conjugate to the epitope of the cell-surface protein (e.g., cell-surface lineage-specific cell-surface protein) induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

Aspects of the disclosure also provide kits, for example kits comprising reagents, e.g., for producing a genetically engineered cell. In some embodiments, the kit comprises a first gRNA and an RNA-guided nuclease that binds the first gRNA. In some embodiments, the first gRNA and RNA-guided nuclease form a ribonucleoprotein (RNP) complex under conditions suitable to bind a first target domain in the genome of a cell or plurality of cells. In some embodiments, the kit comprises a second gRNA and an RNA-guided nuclease that binds the second gRNA. In some embodiments the RNA-guided nuclease that binds the first gRNA is the same as the RNA-guided nuclease that binds the second gRNA. In some embodiments, the RNA-guided nuclease that binds the first gRNA is different from (e.g., distinct from and/or supplied in addition to) the RNA-guided nuclease that binds the second gRNA. In some embodiments, the second gRNA and RNA-guided nuclease form a ribonucleoprotein (RNP) complex under conditions suitable to bind a second target domain in the genome of a cell or plurality of cells.

In some embodiments, the kit comprises instructions for a method of contacting a cell or plurality of cells of with the first gRNA and RNA-guided nuclease and the second gRNA and RNA-guided nuclease, wherein the instructions provide that the cell or plurality of cells is contacted with the first gRNA and RNA-guided nuclease prior to being contacted with the second gRNA and RNA-guided nuclease (e.g., such that a modification to a first target domain is introduced prior to a modification to a second target domain). In some embodiments, the instructions provide for a method that produces a plurality of cells comprising fewer translocation product cells than an otherwise similar method that contacts the cell or plurality of cells with the second gRNA of prior to contacting the plurality of cells with the first gRNA, e.g., as measured by a translocation assay. In some embodiments, the kit comprises a cell or plurality of cells. In some embodiments, the kit does not comprise a cell or plurality of cells (e.g., the cell or plurality of cells recited by the instructions is acquired by other means).

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1: Multiplexed Editing of CD33 and CD19 in CD34+ Hematopoietic Cells

This example demonstrates that the order of treatment of cCD34+ HSCs with multiple genome editing RNPs (one RNP comprising a gRNA targeting a first lineage-specific cell-surface antigen, CD33, and Cas9; and a second RNP comprising a gRNA targeting a second lineage-specific cell-surface antigen, CD19, and Cas9) contributes to the amount of translocation products produced in the process of producing the doubly genetically modified CD34+ HSCs. In particular, this example shows that treatment with CD33 targeted RNPs followed by treatment with CD19 targeted RNPs produces fewer translocation products than either treatment at substantially the same time or the reverse order. The example shows that the order or simultaneousness of treatment had no effect on viability of cells or editing efficiency for either target.

### Methods

Frozen CD34+ HSCs derived from mobilized peripheral blood (mPB) were purchased, for example, from Hemacare or Fred Hutchinson Cancer Center and thawed according to manufacturer's instructions. To edit HSCs, HSCs were thawed and cultured for approximately 40 hours, as shown in FIG. 1, before electroporation with a first RNP and second RNP. The targeting domain sequence of the CD33 and CD19-targeting gRNAs are shown in Table 1.

To electroporate HSCs, 1.5×10⁵ cells were pelleted and resuspended in 20 μL Lonza P3 solution and mixed with 10 μL Cas9 RNP. CD34+ HSCs were electroporated using the Lonza Nucleofector 2 (program DU-100) and the Human P3 Cell Nucleofection Kit (VPA-1002, Lonza). The cells were subjected to a first electroporation with a first RNP and incubated for 30 hours prior to a second electroporation with a second RNP. Cells were harvested 24 and 30 hours following the second electroporation and assessed for viability, on-target editing, and the presence of translocation products. The percentage editing was determined by % INDEL as assessed by TIDE analysis. Editing efficiency was determined by flow cytometric analysis. At varying times post-ex vivo editing, the percentages of viable, edited cells and control cells were quantified using flow cytometry and the 7AAD viability dye.

### Results

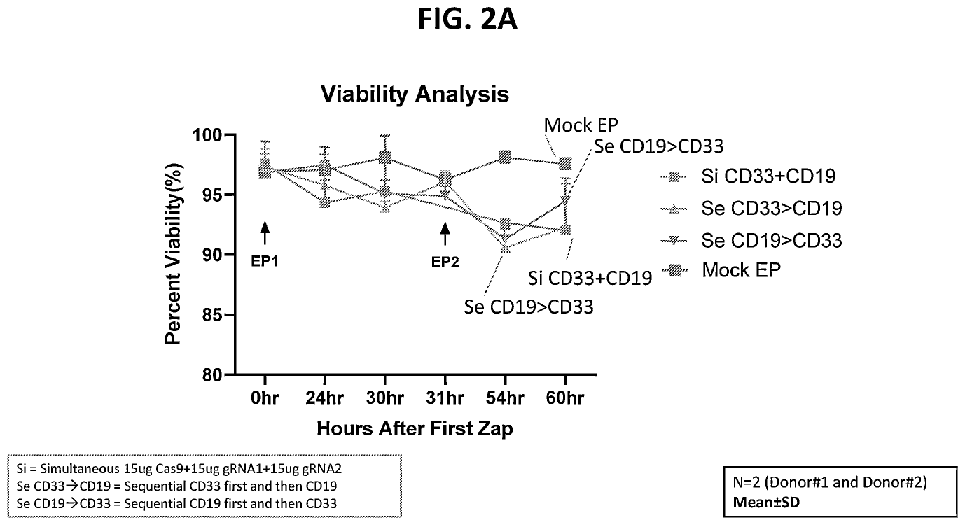

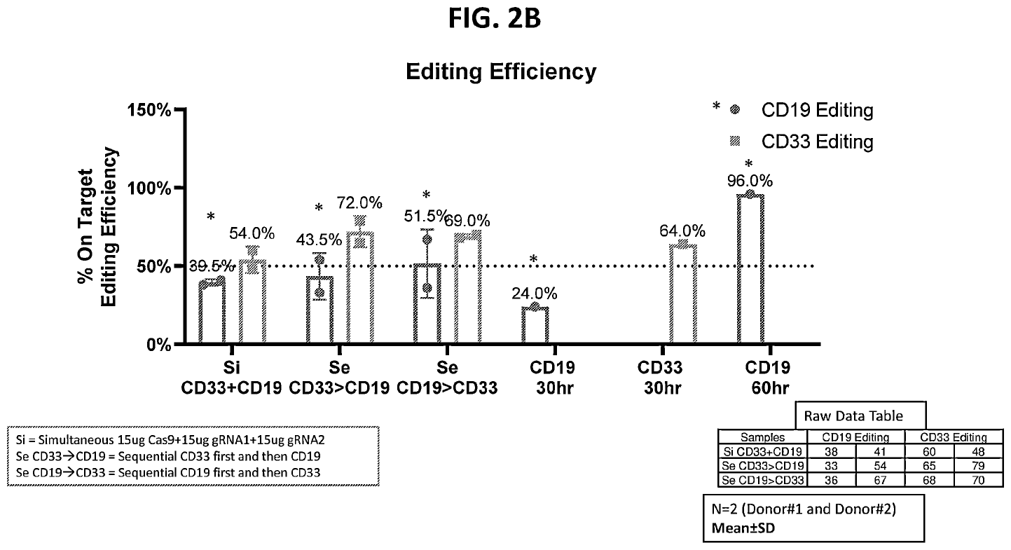

FIG. 2A shows the viability of CD34+ HSCs at the indicated time points following the first electroporation for cells. Groups of cells were electroporated with RNPs targeting CD33 and CD19 at the same time (Si CD33+CD19); cells treated sequentially with CD33 targeted RNPs first followed by CD19 targeted RNPs second (Se CD33>CD19), cells treated sequentially with CD19 targeted RNPs first followed by CD33 targeted RNPs second (Se CD19>CD33), or mock electroporated. No significant difference in viability was observed based on the order or simultaneousness of electroporation.

FIG. 2B shows the editing efficiency for CD33 editing and CD19 editing in each of the groups of cells. The results show that there is no significant difference in editing efficiency based upon order of treatment.

As discussed herein, genetic editing, for example involving the generation of double strand breaks, may result in the production of translocation products. See, FIG. 3. Translocation products produced by DNA repair events between the double strand breaks produced by the CD33 targeted RNP and the CD19 targeted RNP can be predicted to fall into certain categories (see, FIG. 3). Primer pairs were selected to detect particular translocation products using PCR analysis, the approximate location of each shown in FIG. 3. The products of those PCR reactions were analyzed qualitatively via gel electrophoresis and quantitatively using a ddPCR assay.

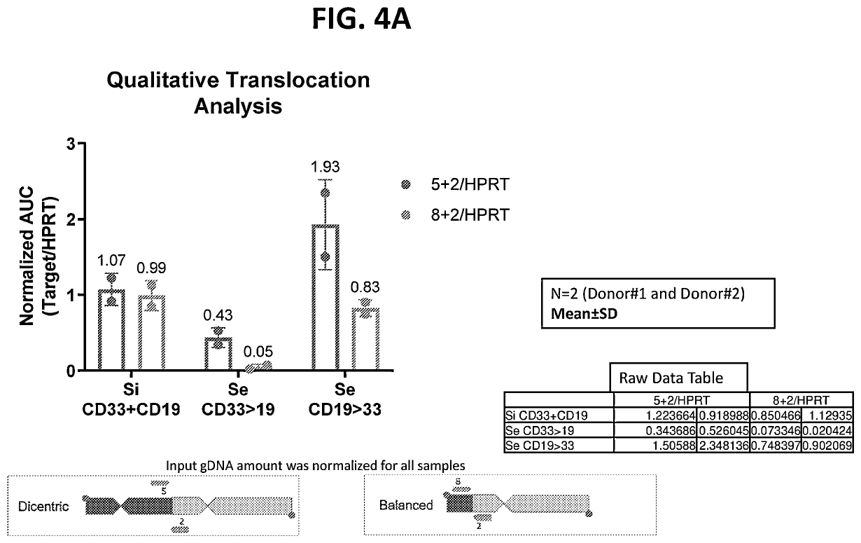

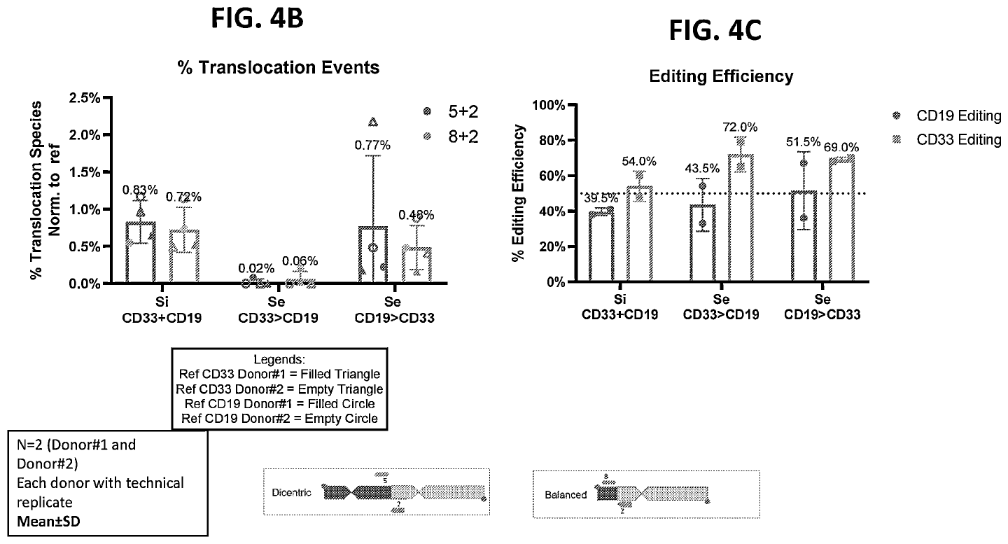

FIG. 4A shows results of a qualitative translocation analysis of translocation products (in this experiment, dicentric and balanced translocation products, using primer pairs 5 and 2 or 8 and 2, respectively). Significantly fewer translocation products were detected when cells were electroporate with CD33 targeted RNPs followed by CD19 targeted RNPs as compared to the reverse order or with treatment at substantially the same time. FIG. 4B shows results of a quantitative translocation analysis (% translocation events) of translocation products by ddPCR and, consistent with the qualitative analysis (FIG. 4A), showed that significantly fewer translocation products were detected when cells were electroporated with CD33 targeted RNPs followed by CD19 targeted RNPs than the reverse order or with treatment at substantially the same time. The analysis also showed that editing efficiency was unaffected by the sequence or simultaneousness of treatment. See, FIG. 4C.

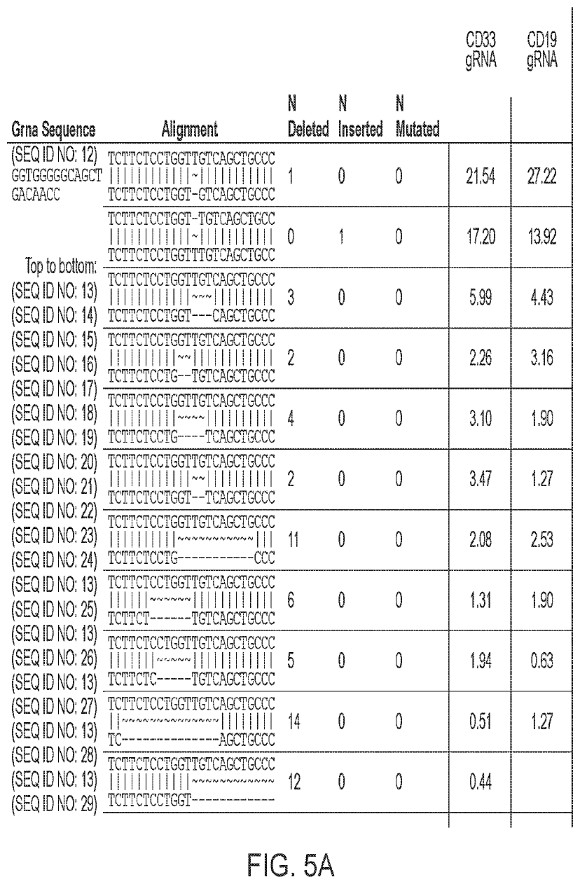

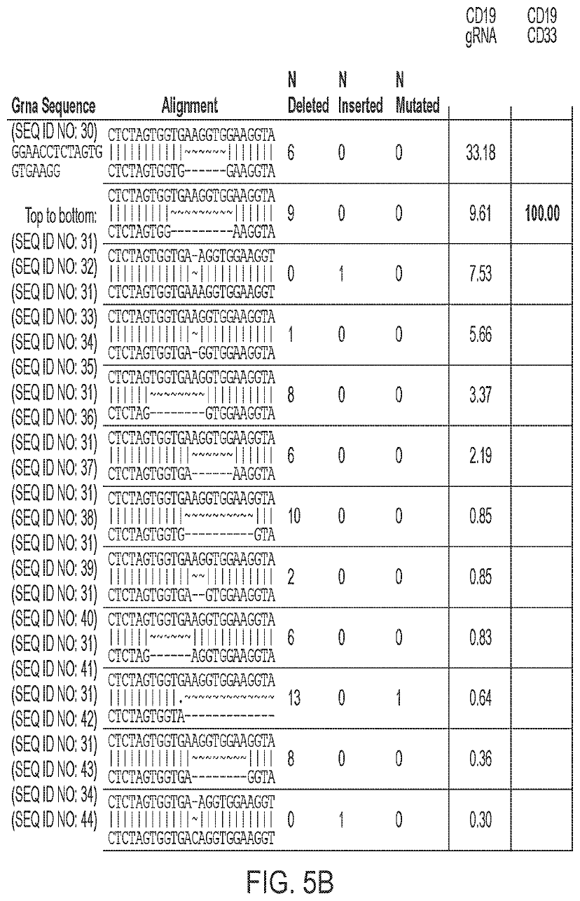

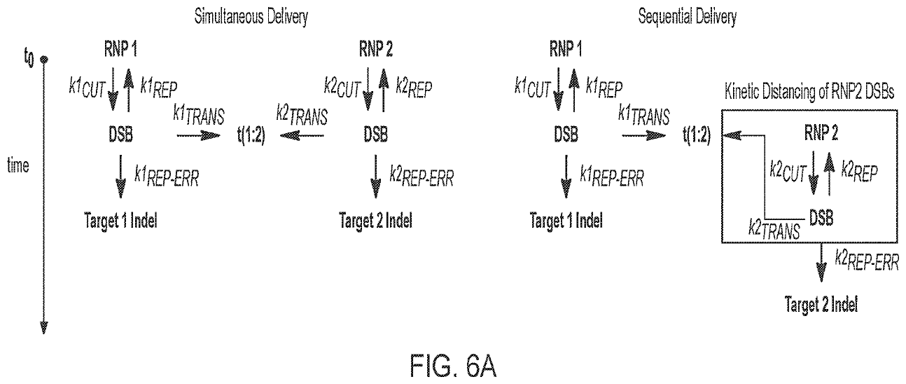

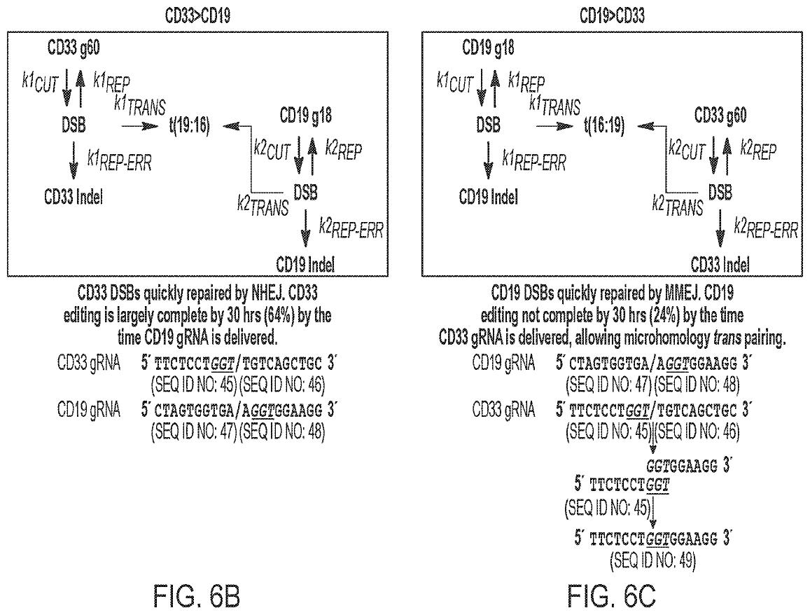

The resulting products from editing with the CD33 targeted RNPs and CD19 targeted RNPs are shown in FIGS. 5A and 5B, respectively, showing the positions of the insertions or deletions (indels) detected after treatment with RNPs. The indel data showed that targeting CD33 with g60 gRNA produced a high number of −1 position indels, which suggests the double strand breaks associated with those indels were recognized and repaired by non-homologous end joining (NHEJ). In contrast, the indel data showed that targeting CD19 with g18 gRNA produced a high number of −6 and −9 position indels, which suggests the double strand breaks associated with those indels were recognized and repaired by microhomology mediated end joining (MMEJ). Without wishing to be bound by theory, a double strand break recognized primarily by NHEJ may be repaired faster than a double strand break recognized primarily by non-NHEJ mechanisms (e.g., MMEJ). It is hypothesized (see FIGS. 6A-6C) that first inducing a double strand break recognized primarily by NHEJ followed by a double strand break recognized primarily by non-NHEJ mechanisms (e.g., MMEJ), the first double strand break may be substantially repaired (e.g., to form an indel) prior to the occurrence of the second double strand break, thus decreasing the amount of or preventing formation of translocation products from the two double strand breaks.

### Example 2: Multiplexed Editing of CD33 and CD5 in CD34+ Hematopoietic Cells

This example demonstrates that the order of treatment of CD34+ HSCs with multiple genome editing RNPs (one RNP comprising a gRNA targeting a first lineage-specific cell-surface antigen, CD33, and Cas9; and a second RNP comprising a gRNA targeting a second lineage-specific cell-surface antigen, CD5, and Cas9) is important to the level of translocation products produced in the process of producing the doubly genetically modified CD34+ HSCs. In particular, this example shows that treatment with CD5 targeted RNPs followed by treatment with CD33 targeted RNPs was particularly favorable with regard to observed translocation frequency. The example shows that the order or simultaneousness of treatment had no effect on viability of cells or editing efficiency for either target. Additionally, this example shows successful engraftment and differentiation of multiplex-edited cells into an immunodeficient mouse model (NOD.Cg-Prkdc^{scid}Il2rg^{tm1Wjl}/SzJ (also known as “NOD scid gamma” or “NSG”) mice).

### Methods

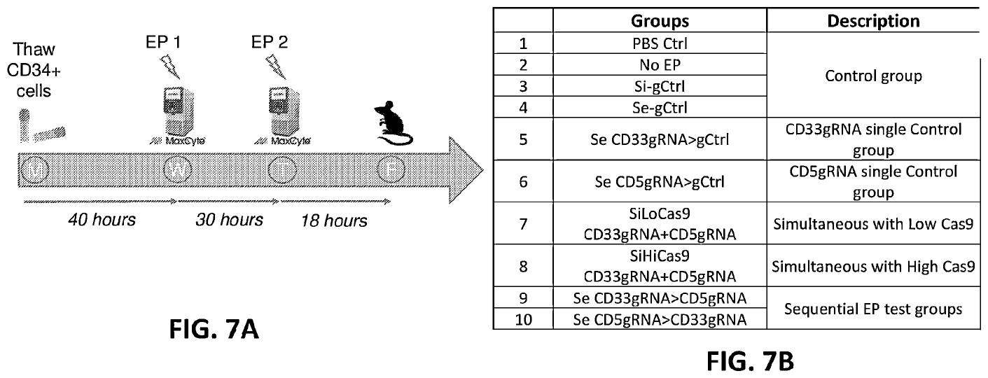

Frozen CD34+ HSCs derived from mobilized peripheral blood (mPB) were purchased, for example, from Hemacare or Fred Hutchinson Cancer Center and thawed according to manufacturer's instructions. HSCs were thawed and cultured for approximately 40 hours, as shown in FIG. 7A, before electroporation with a first RNP and second RNP. The targeting domain sequence of the CD33 and CD5-targeting gRNAs are shown in Table 1. The targeting domain sequence of the control gRNA (gCtrl) for use with the Cas9 nuclease (SpyCas9) is provided below.

gCtrl (SpyCas9)
(SEQ ID NO: 9)
GCCGACGCGAAATCTTAGCGNRG

To electroporate HSCs, cells were pelleted and resuspended in Lonza P3 solution and mixed with Cas9 RNP. CD34+ HSCs were electroporated using the Lonza Nucleofector 2 (program DU-100) and the Human P3 Cell Nucleofection Kit (VPA-1002, Lonza). The cells were subjected to a first electroporation with a first RNP and incubated for 30 hours prior to a second electroporation with a second RNP. Cells were harvested 18 hours following the second electroporation and assessed for viability, on-target editing, the presence of translocation products. The percentage editing was determined by % INDEL as assessed by TIDE analysis. Editing efficiency was determined by flow cytometric analysis. At varying times post-ex vivo editing, the percentages of viable, edited cells and control cells were quantified using flow cytometry and the 7AAD viability dye. The groups of cells and respective treatments are shown in FIG. 7B and experimental parameters shown in Table 3.

After electroporation of the second RNP, cells were injected into 3-week-old NSG mice that had been treated with 200 centigray (cGy) irradiation. Recipient NSG mice were irradiated with full-body gamma radiation before injection with the modified HSCs. 1×10⁵ CD34+ cells per injection were injected into the lateral tail vein of each mouse. Sixteen (16) weeks after injection with HSCs, human chimerism of the recipient mice's bone marrow is assessed by flow cytometry.

TABLE 3

| | | | | # cells (w/ | |
|---|---|---|---|---|---|
| | | | | 50% | |
| Study group | N | Cas9 | gRNA | overage) | Radiation |
| Control (PBS) | 3 | — | — | — | — |
| Culture alone | 5 | — | — | 4.5M | 200 cGy |
| gCtrl EP + | 8 | 15 μg Cas9/ | 15 μg gCtrl/ | 12M | 200 cGy |
| gCtrl EP (30 | | 15 μg Cas9 | 15 μg gCtrl | | |
| hr gap) | | | | | |
| CD33 gRNA | 6 | 15 μg Cas9/ | 15 μg CD33 | 12M | 200 cGy |
| single + | | 15 μg Cas9 | gRNA/15 μg | | |
| mock/gCtrl | | | gCtrl | | |
| EP | | | | | |
| CD5 gRNA | 8 | 15 μg Cas9/ | 15 μg CD5 | 12M | 200 cGy |
| single + | | 15 μg Cas9 | gRNA/ | | |
| mock/gCtrl | | | 15 μg gCtrl | | |
| EP | | | | | |
| RNP 1 + 2 | 7 | 15 μg Cas9 | 15 μg CD33 | 12M | 200 cGy |
| (Si, low Cas9) | | | gRNA/15 μg | | |
| | | | gCD5 gRNA | | |
| RNP 1 + 2 | 7 | 30 μg Cas9 | 15 μg CD33 | 12M | 200 cGy |
| (Si, high ) | | | gRNA/15 μg | | |
| Cas9 | | | gCD5 gRNA | | |
| RNP 1→2 | 8 | 15 μg Cas9 | 15 μg CD33 | 12M | 200 cGy |
| (seq 1) (30 hr | | | gRNA/15 μg | | |
| gap) | | | gCD5 gRNA | | |
| RNP 2→1 | 8 | 15 μg Cas9 | 15 μg CD5 | 12M | 200 cGy |
| (seq 2) (30 hr | | | gRNA/15 μg | | |
| gap) | | | gCD33 gRNA | | |

### Results

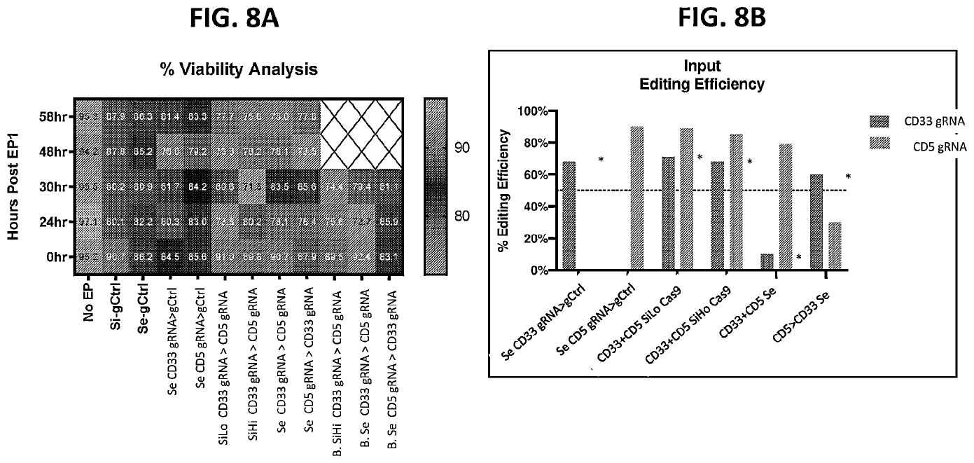

FIG. 8A shows the viability of CD34+ HSCs at the indicated time points following the first electroporation for cells. All groups had more than 70% viability at the time of injection of the genetically modified cells to the mice. No significant difference in viability was observed based on the order or simultaneousness of electroporation.

FIG. 8B shows editing efficiency for the CD33 editing and CD5 editing in each of the groups of cells. The results show that there was a slight increase in on-target editing efficiency in cells that were simultaneously electroporated with the two RNPs as compared to sequential editing.

As discussed above in Example 1, primer pairs were selected to detect particular translocation products using PCR analysis, the approximate location of each shown in the schematic on the right in FIG. 9. The products of those PCR reactions were analyzed qualitatively via gel electrophoresis and quantitatively using a ddPCR assay.

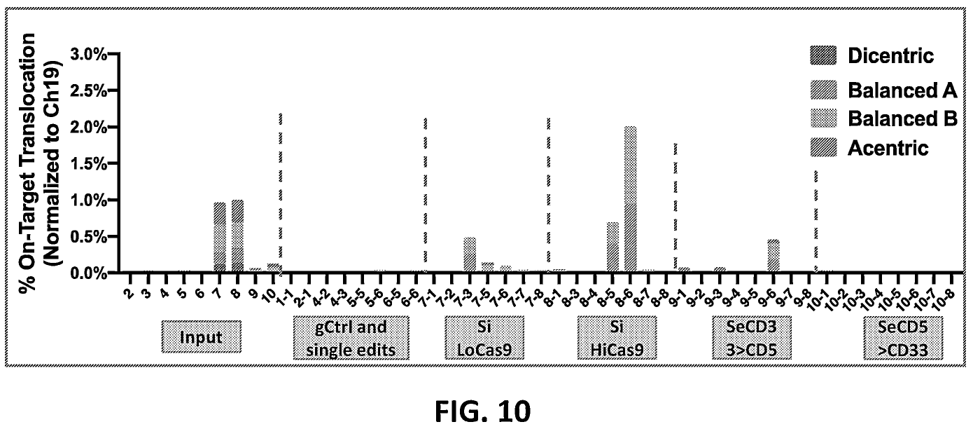

FIG. 9 shows the percent on-target translocation products, indicating the relative amount of each of the types of translocation product observed. Fewer translocation products were detected in the input cells (edited cells to be injected into NSG mice) when cells are treated sequentially with the two RNPs as compared to electroporation with the two RNPs at substantially the same time. FIG. 10 shows the percent on-target translocation products (as normalized to chromosome 19), indicating the relative amount of each of the types of translocation product observed. The “input” samples refers to cells that have been electroporated and are to be injected into mice. The other indicated groupings correspond to cells harvested from the various groups of animals and then analyzed. For example, the input from group 9 (SeCD3>CD5) was found to have lower incidences of translocations (input “9”) as compared to the input for group 10 (SeCD5>CD33). However, cells harvested after transplantation (group 9 SeCD33>CD5) there appeared to be some animals with persistent translocations (e.g., 9-1, 9-3, and 9-6), whereas cells harvested from group 10 (SeCD5>CD33) animals did not appear to have maintained any translocations. Without wishing to be to be bound by any particular theory, these results may suggest that any translocations that occurred (as measured in the input), may have been selected against in the animals. In general, fewer translocation products were detected in the input sample (cells injected into NSG mice) when cells are treated sequentially with the two RNPs as compared to electroporation with the two RNPs at substantially the same time. There was a slight decrease in the amount of translocation products detected in cells that were sequentially electroporated with the CD5 targeting RNPs followed by the CD33 targeting RNPs, as compared to cells that were sequentially electroporated with the CD33 targeting RNPs followed by the CD5 targeting RNPs,

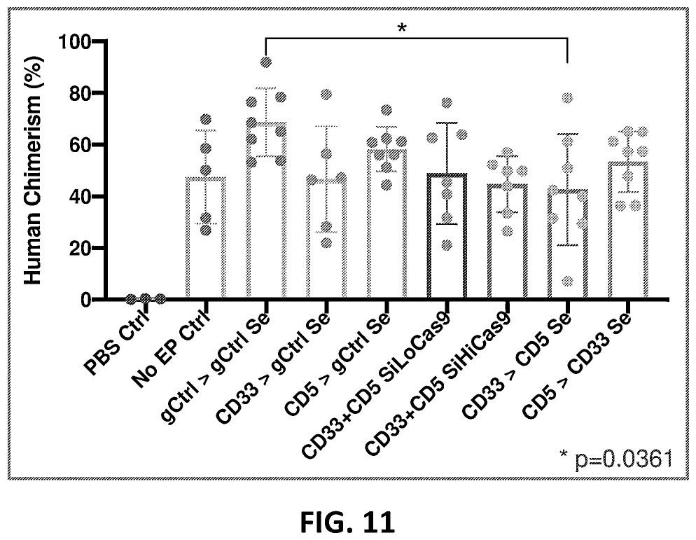

Sixteen weeks following injection of edited CD34+ cells into NSG mice, cells were analyzed for human chimerism, as an indicator of engraftment of the edited cells. The results indicated that CD34+ cell fitness was not affected by Cas9 multiplexing electroporation or CD33 and CD5 editing. See, FIG. 11.

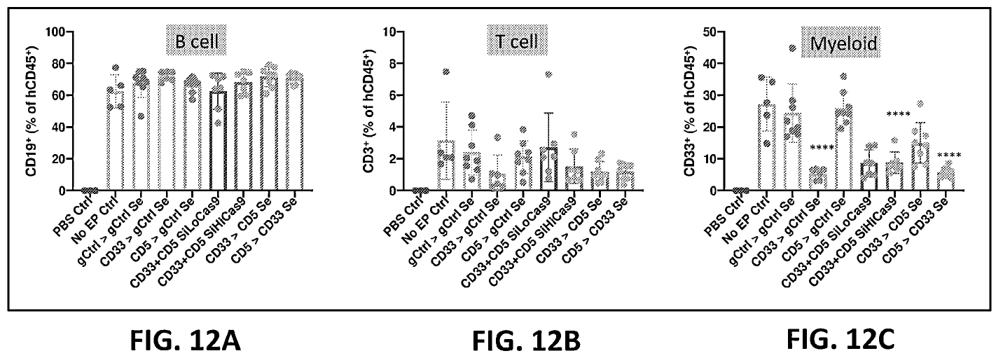

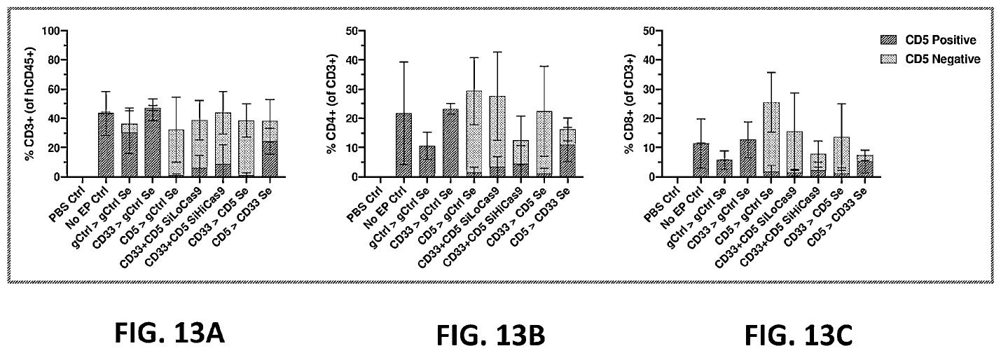

Cells were further assessed to determine whether the multiplexed editing impacted the cells' ability to differentiate into different cellular lineages. FIGS. 12A-12C show B and T cell lineages are not affected by multiplex gene editing (no sequential versus simultaneous editing), while the percentage of myeloid-lineage cells (hCD33+) was low due to the removal of CD33 by gene editing. Finally, subsets of T cell progenitor cells were also assessed. FIGS. 13A-13C show detection of T cell progenitor cells in this experiment despite that the mouse line has under-developed thymi. The CD5-edited groups showed lower levels of CD5 protein expression, as expected, and did have detectable levels of CD4+ and CD8+ cells.

### Example 3: Multiplexed Editing of CD33 and CD19 in CD34+ Hematopoietic Cells Using Cas9 and Cpf1 Nuclease

This example demonstrates that the order of treatment of CD34+ HSCs with multiple genome editing RNPs (one RNP comprising a gRNA targeting a first lineage-specific cell-surface antigen, CD33, and Cas9; and a second RNP comprising a gRNA targeting a second lineage-specific cell-surface antigen, CD19, and Cpf1) contributes to the amount of translocation products produced in the process of producing genetically modified CD34+ HSCs edited using Cas9 and Cpf1. In particular, this example shows that treatment with sequential editing (using Cas9 and Cpf1) produces fewer translocation products than editing at substantially the same time. The example shows that the order or simultaneousness of treatment had no effect on viability of cells or editing efficiency for either target.

### Methods

Frozen CD34+ HSCs derived from mobilized peripheral blood (mPB) were purchased, for example, from Hemacare or Fred Hutchinson Cancer Center and thawed according to manufacturer's instructions. To edit HSCs, HSCs were thawed and cultured for approximately 40 hours, as shown in FIG. 1, before electroporation with a first RNP and second RNP. The targeting domain sequence of the CD33 and CD19-targeting gRNAs are shown in Table 1.

To electroporate HSCs, 1.5×10⁵ cells were pelleted and resuspended in 20 μL Lonza P3 solution and mixed with 10 μL Cas9 RNP. CD34+ HSCs were electroporated using the Lonza Nucleofector 2 (program DU-100) and the Human P3 Cell Nucleofection Kit (VPA-1002, Lonza). The cells were subjected to a first electroporation with a first RNP and incubated for 30 hours prior to a second electroporation with a second RNP. Cells were harvested 24 and 30 hours following the second electroporation and assessed for viability, on-target editing, and the presence of translocation products. The percentage editing was determined by % INDEL as assessed by TIDE analysis. Editing efficiency was determined by flow cytometric analysis. At varying times post-ex vivo editing, the percentages of viable, edited cells and control cells were quantified using flow cytometry and the 7AAD viability dye.

### Results

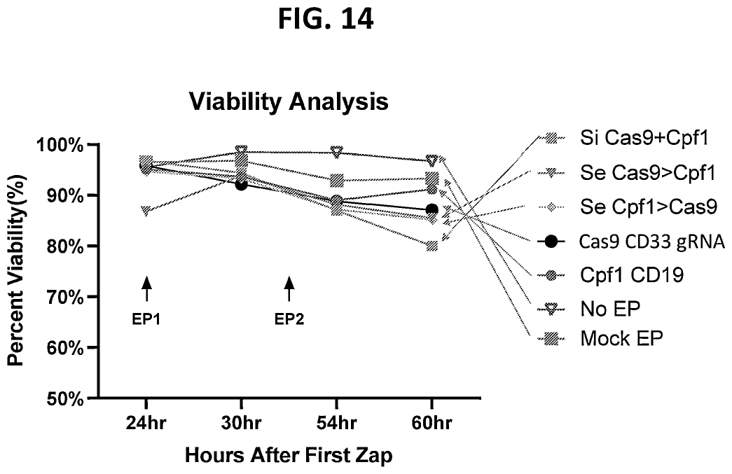

FIG. 14 shows the viability of CD34+ HSCs at the indicated time points following the first electroporation for cells. Groups of cells were electroporated with RNPs targeting CD33 and Cas9 nuclease and RNPs targeting CD19 and Cpf1 at the same time (Si Cas9+Cpf1); cells treated sequentially with RNPs targeting CD33 and Cas9 first followed by RNPs targeting CD19 and Cpf1 second (Se Cas9>Cpf1), cells treated sequentially with RNPs targeting CD19 and Cpf1 first followed by RNPs targeting CD33 and Cas9 second (Se Cpf1>Cas9, either of the single RNPs (Cas9 CD33 or Cpf1 CD19), no electroporation, or mock electroporated. No significant difference in viability was observed based on the order or simultaneousness of electroporation.

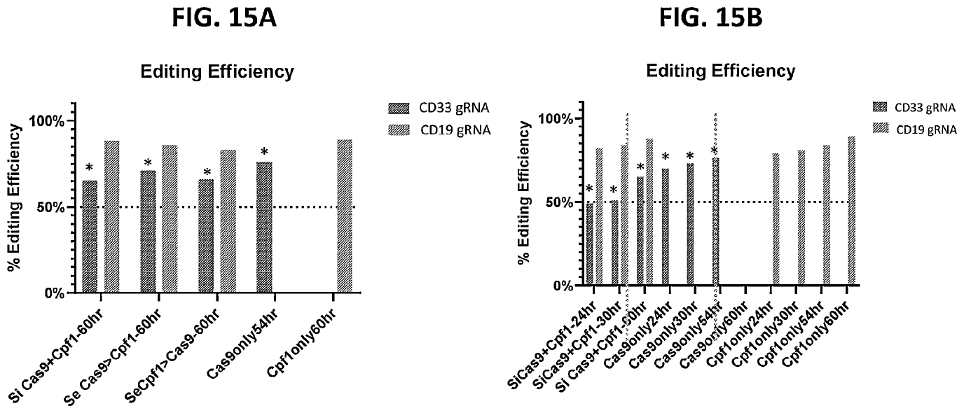

FIGS. 15A and 15B show the editing efficiency for CD33 editing and CD19 editing in each of the groups of cells. The results show that there is no significant difference in editing efficiency based upon order of treatment.

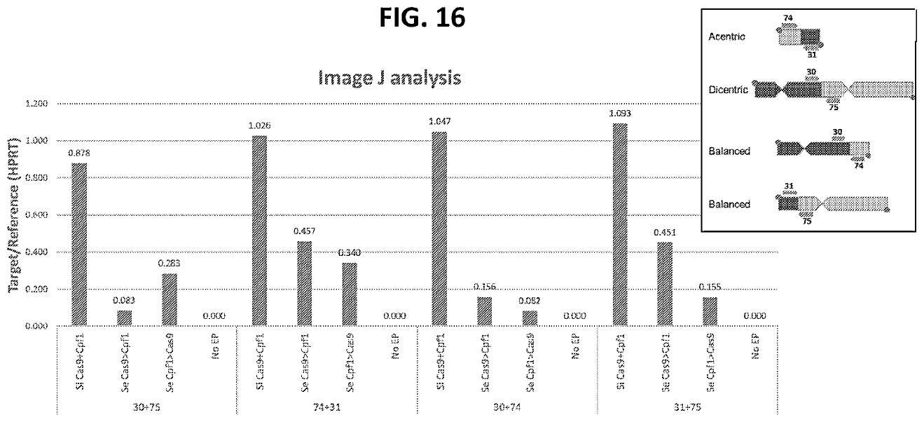

As discussed herein, genetic editing, for example involving the generation of double strand breaks, may result in the production of translocation products. See, FIG. 3. Translocation products produced by DNA repair events between the double strand breaks produced by the CD33 targeted RNP and the CD19 targeted RNP can be predicted to fall into certain categories (see, FIG. 3). Primer pairs were selected to detect particular translocation products using PCR analysis, as shown in the right panel of FIG. 16. The products of those PCR reactions were analyzed qualitatively via gel electrophoresis and quantitatively using a ddPCR assay.

FIG. 16 shows quantification (by ImageJ software) of PCR products from PCR reactions using each of the indicated pairs of primers. For each of the translocation product species assess, fewer translocation products were detected when cells were electroporated sequentially with RNPs as compared to electroporation simultaneously. A slight reduction in most of the translocation products produced in cells electroporated first with RNPs targeting CD19 and Cpf1 followed by RNPs targeting CD33 and Cas9 as compared to the reverse order.

### Example 4: Treatment of Hematologic Disease

An example treatment regimen using the methods, cells, and agents described herein for acute myeloid leukemia is provided below.
1) Identify a patient with AML that is a candidate for receiving a hematopoietic cell transplant (HCT);
2) Identify a HCT donor with matched HLA haplotypes, using standard methods and techniques;
3) Extract the bone marrow from the donor;
4) Genetically manipulate the donor bone marrow cells ex vivo. Briefly, sequentially introduce targeted modifications (deletion, substitution) of a lineage-specific cell-surface antigen using a gRNA and a CRISPR/Cas nuclease (e.g., Cas9, Cpf1), as described herein. Cells may be evaluated for characteristics to determine their ability to differentiate and the ability to engraft the patient and mediate graft-vs-tumor (GVT) effects.

### Optional Steps 5-7:

In some embodiments, Steps 5-7 provided below may be performed (once or multiple times) in an exemplary treatment method as described herein:
5) Pre-condition the AML patient using standard techniques, such as infusion of chemotherapy agents (e.g., etoposide, cyclophosphamide) and/or irradiation;
6) Administer the engineered donor bone marrow to the AML patient, allowing for successful engraftment;
7) Follow up with a cytotoxic agent, such as immune cells expressing a chimeric receptor (e.g., CAR T cell) or antibody-drug conjugate, wherein the epitope to which the cytotoxic agent binds is the same epitope that was modified and is no longer present on the donor engineered bone marrow graft. The targeted therapy should thus specifically target the lineage-specific cell surface antigen, without simultaneously eliminating the bone marrow graft, in which the epitope is not present.

### Optional Steps 8-10:

In some embodiments, Steps 8-10 may be performed (once or multiple times) in an exemplary treatment method as described herein:
8) Administer a cytotoxic agent, such as immune cells expressing a chimeric receptor (e.g., CAR T cell) or antibody-drug conjugate that targets an epitope of a lineage specific cell surface antigen. This targeted therapy would be expected to eliminate both cancerous cells as well as the patient's non-cancerous cells;
9) Pre-condition the AML patient using standard techniques, such as infusion of chemotherapy agents;
10) Administer the engineered donor bone marrow to the AML patient, allowing for successful engraftment.

The steps 8-10 result in the elimination of the patient's cancerous and normal cells expressing the targeted protein, while replenishing the normal cell population with donor cells that are resistant to the targeted therapy.

### Example 5: Multiplexed Editing of CLL-1 and CD33 in Human Hematopoietic Stem Cells

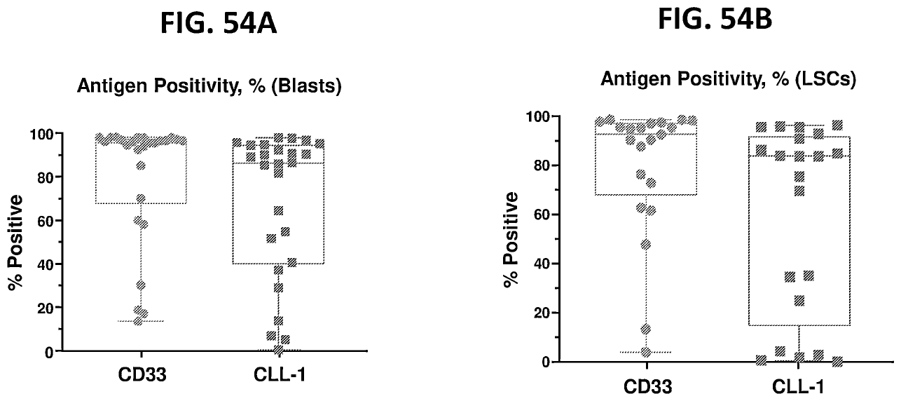

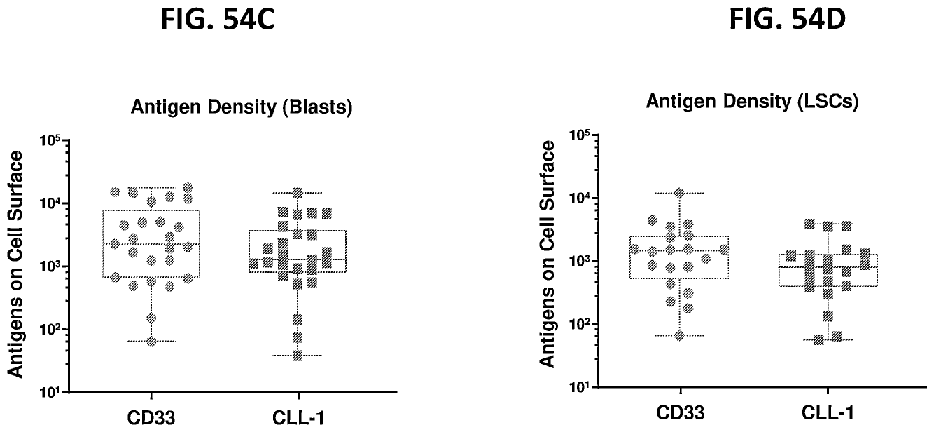

CLL-1 and CD33 are highly expressed in AML patient-derived blasts/leukemic stem cells (LSCs). See, FIGS. 54A-54D. Targeting these antigens, however, can lead to cytopenia due to shared expression on normal hematopoietic cells. This example demonstrates multiplexed editing of human HSCs to generate cells having reduced or eliminated expression of CD33 and CLL-1.

### Methods

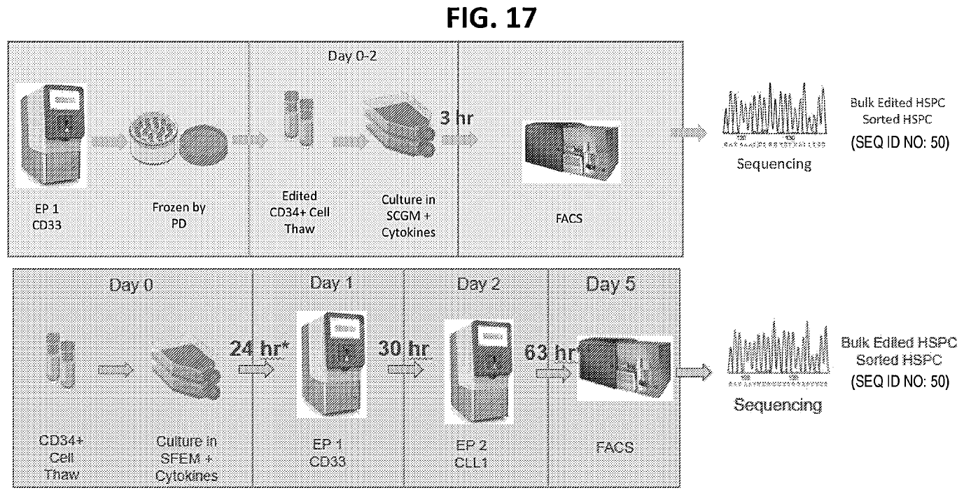

For multiplexed editing, human HSCs (e.g., CD34+ cells) were thawed and cultured in SFEM supplemented with cytokines for 24 hours. Then, cells were electroporated with a ribonucleoprotein complex comprising a first gRNA and a CRISPR-Cas nuclease (referred to as “EP1”). Cells were incubated for 30 hours prior to a second electroporation step with a second ribonucleoprotein complex comprising a second gRNA and a CRISPR-Cas nuclease (referred to as “EP2”). After culturing for 63 hours, cells were harvested, sorted using flow cytometry, and subjected to sequencing analyses. See, FIG. 17.

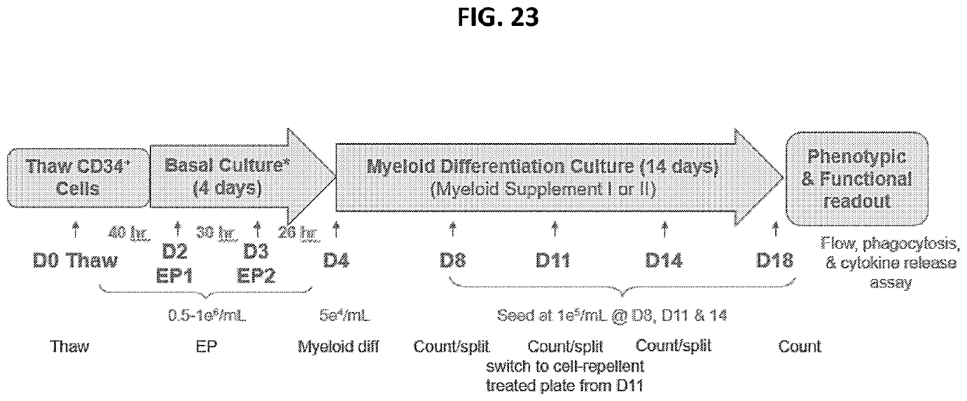

For myeloid differentiation studies, human HSCs (e.g., CD34+ cells) were thawed and cultured for 40 hours. Flow cytometry analysis was used to confirm expression of CLL-1 and CD33 on days 0, 1, and 2. Then, cells were prepared at a concentration of 0.5×10⁶ cells/mL to 1×10⁶ cells/mL for sequential electroporation procedures as discussed above. Here, cells were incubated for 30 hours between EP1 and EP2. After 26 hours post-EP2, cells were prepared at a concentration 5×10⁴ cells/mL and incubated for 4 days in supplemented media to promote myeloid differentiation. Cells were cultured in myeloid differentiation media (e.g., Myeloid Supplement I or Myeloid Supplement II from STEMCELL Technologies) for 14 days, during which on days 8, 11, and 14, cells were counted, split, and seeded at a concentration of 1×10⁵ cells/mL. On day 11, cells were switched to cell-repellent treated plates and maintained until day 18. On day 18, cells were harvested and subjected to phenotypic and functional assays, such as phagocytosis and cytokine release assays, to characterize differentiation. Throughout the experiment on days 3, 4, 5, 6, 8, 11, 14, and 18, cell samples were used for flow cytometry analysis of CD33 and CLL-1 expression levels and cell pellets were harvested for downstream gDNA and transcript analyses. See FIG. 23.

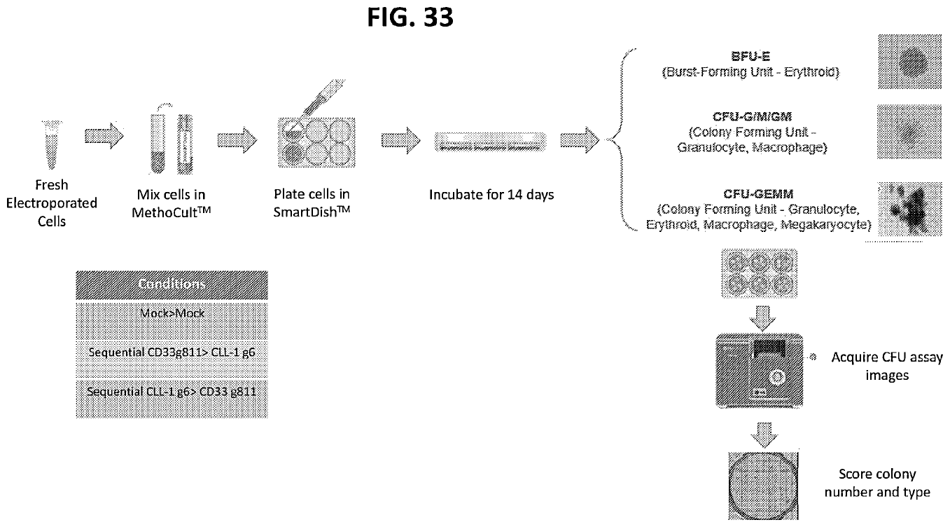

To assess lineage differentiation, electroporated cells were mixed with MethoCult™ and plated in a SmartDish™ in duplicates at densities of 200 cells/well and 300 cells/well across a total of 3 plates. Cells were incubated for 14 days prior to colony-forming unit (CFU) imaging and scoring. See FIG. 33.

### Results

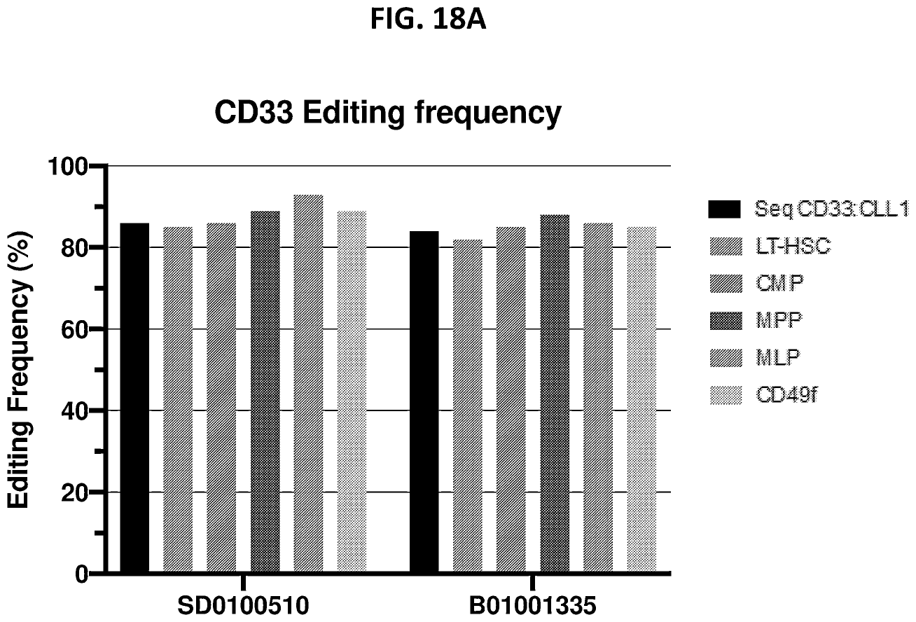

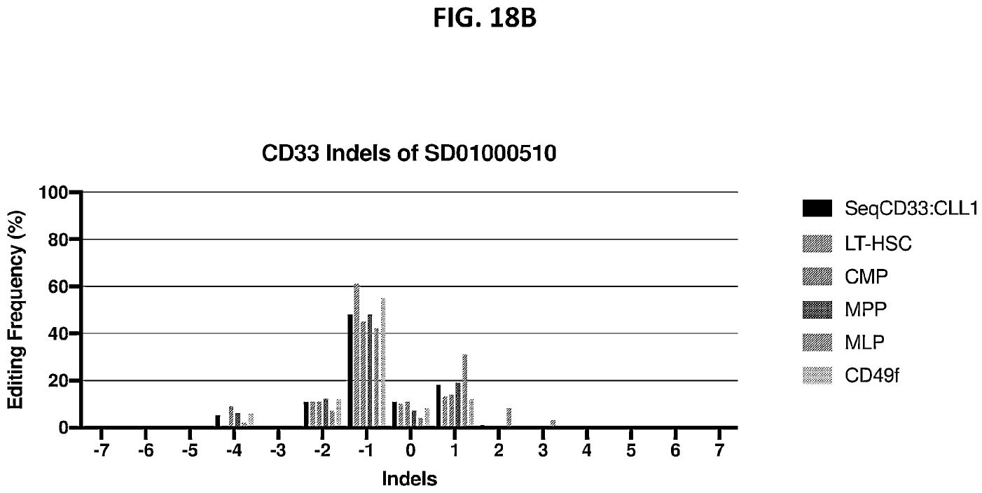

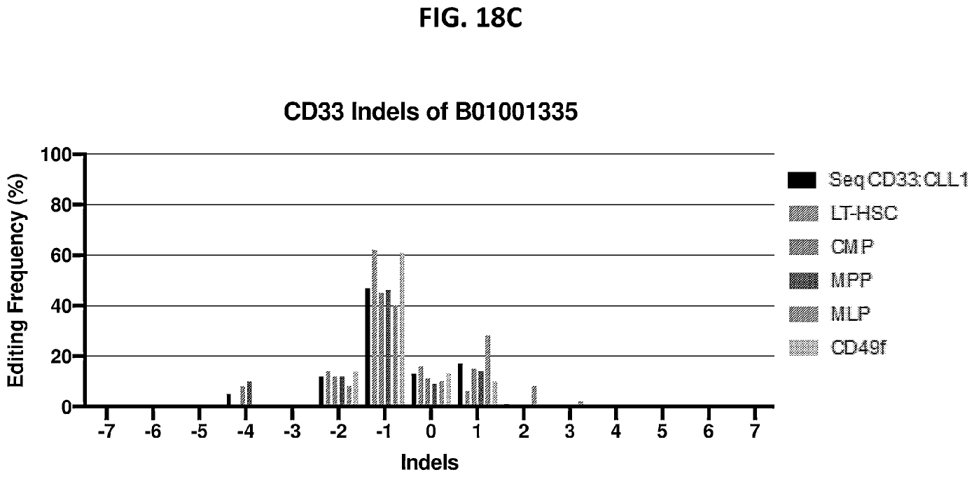

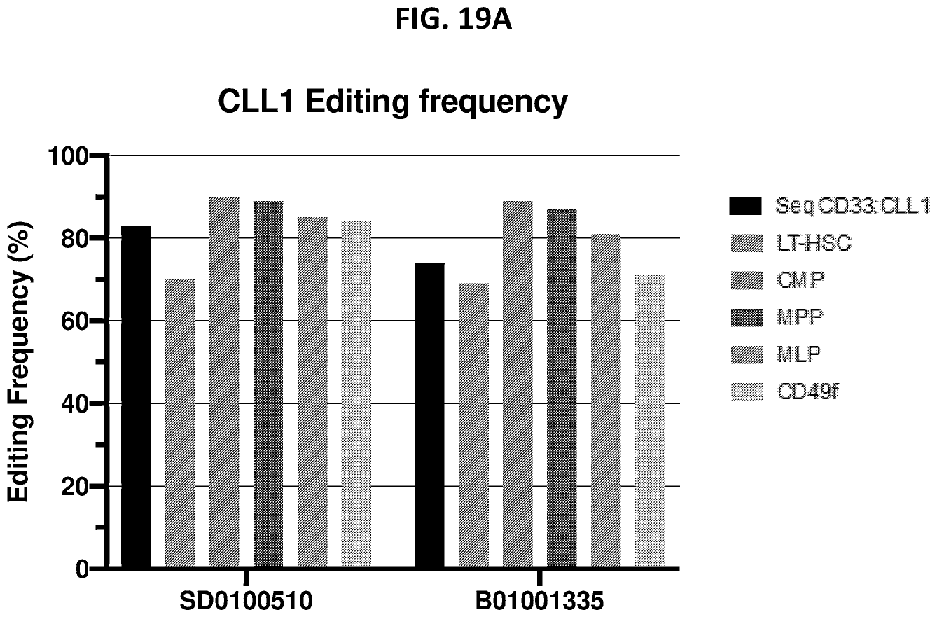

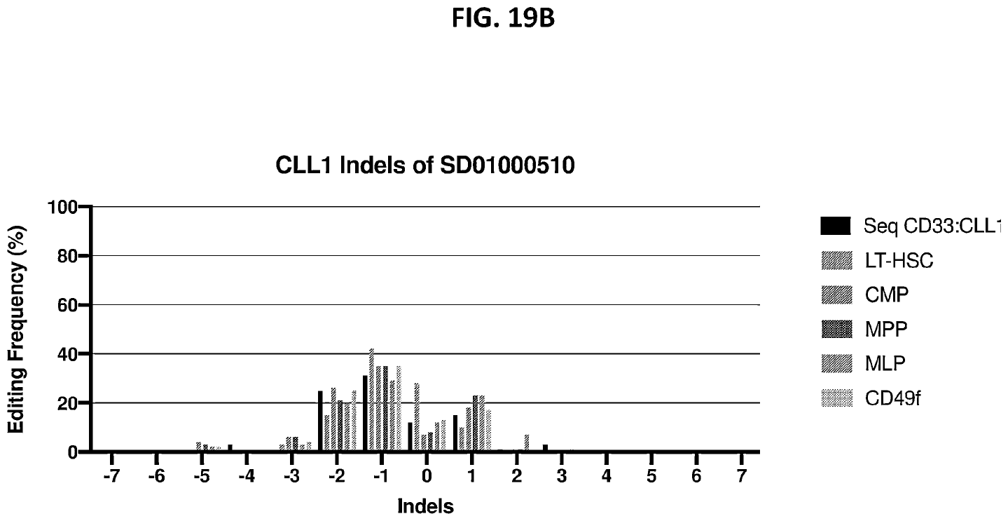

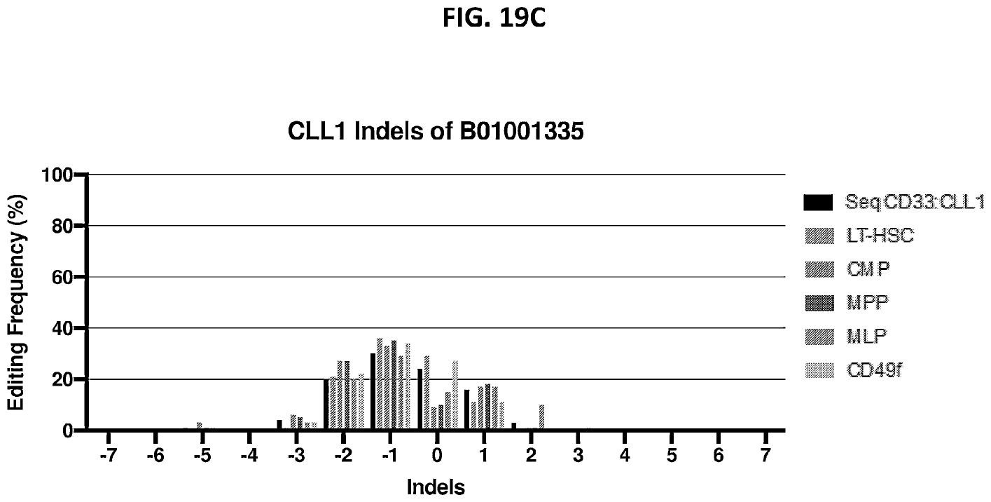

Multiplex edited cells were sorted into subpopulations to evaluate editing frequencies in the various cell types: LT-HSC, CMP, MPP, MLP, and CD49f. FIGS. 18A-18C and FIGS. 19A-19C show CD33 and CLL-1 editing frequency in human HSCs derived from two independent bone marrow donors, respectively. The editing frequency of both CD33 and CLL-1 was found to be comparable between the cell subpopulations and comparable to editing frequency observed in the bulk edited cell population.

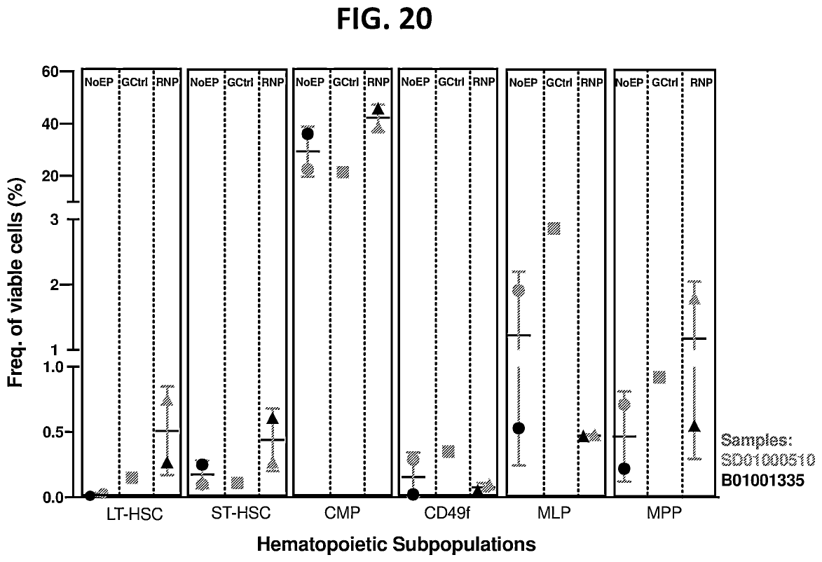

FIG. 20 shows viability analysis of multiplex edited bone marrow cells, which indicated that the multiplex edited human HSCs exhibited similar viability levels as control cells.

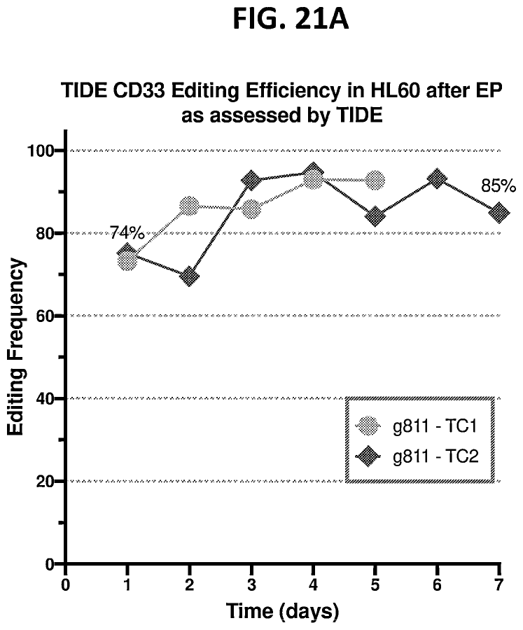

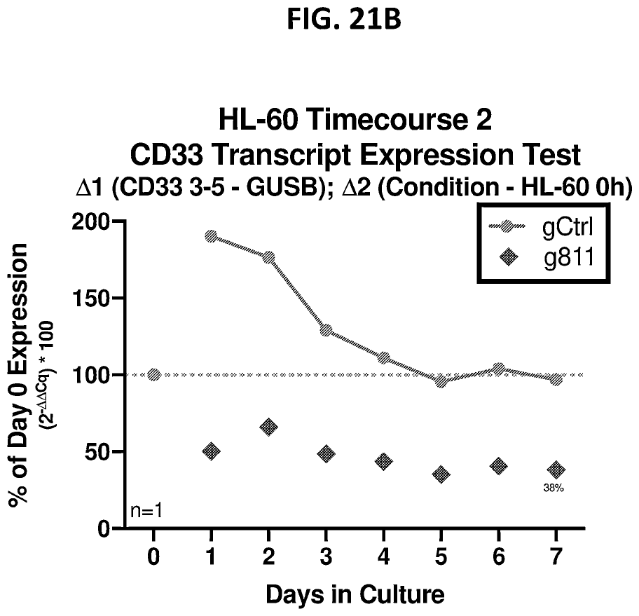

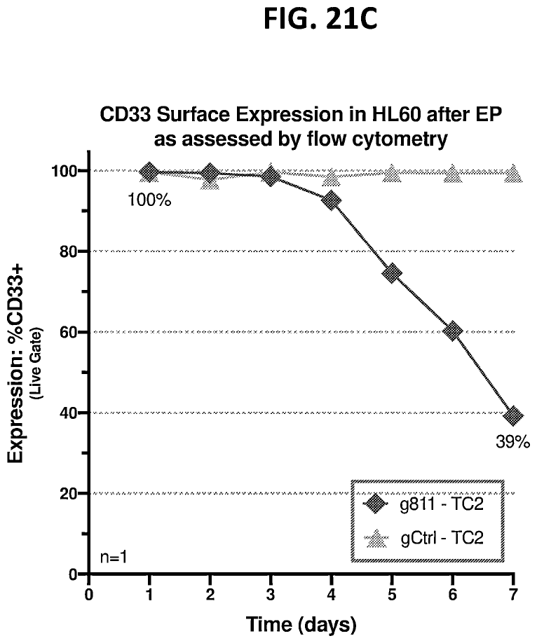

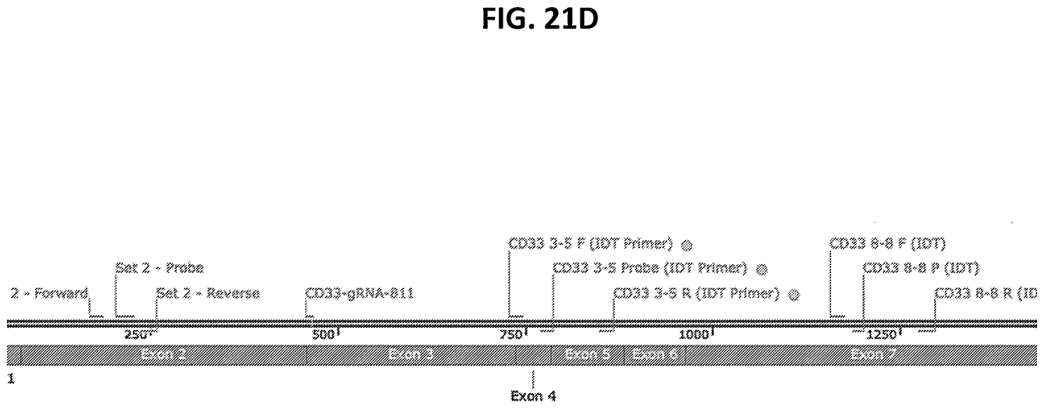

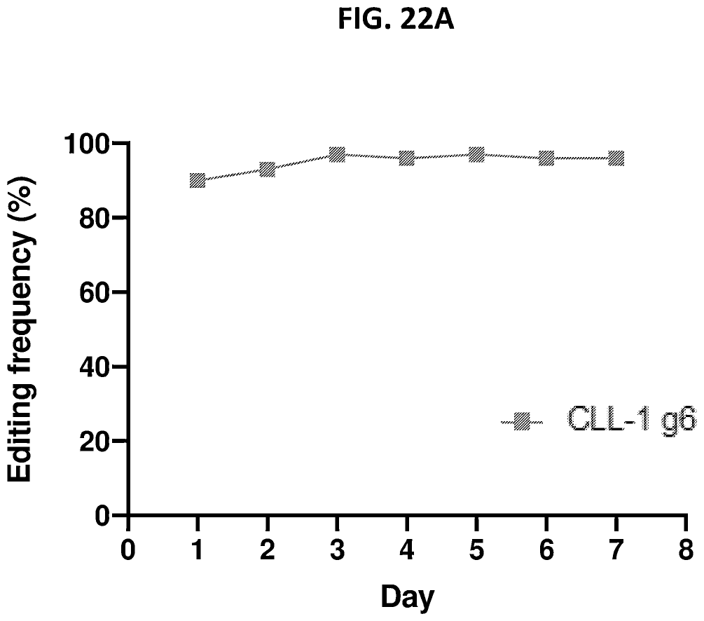

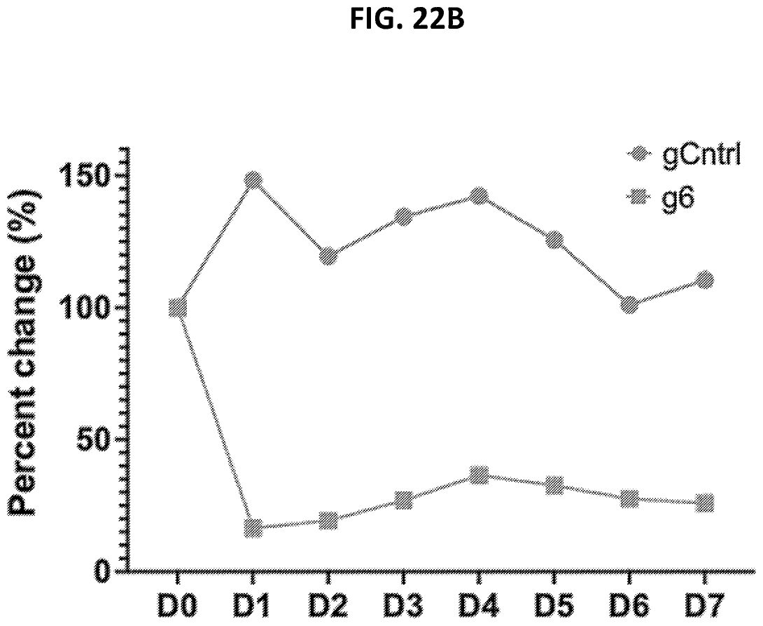

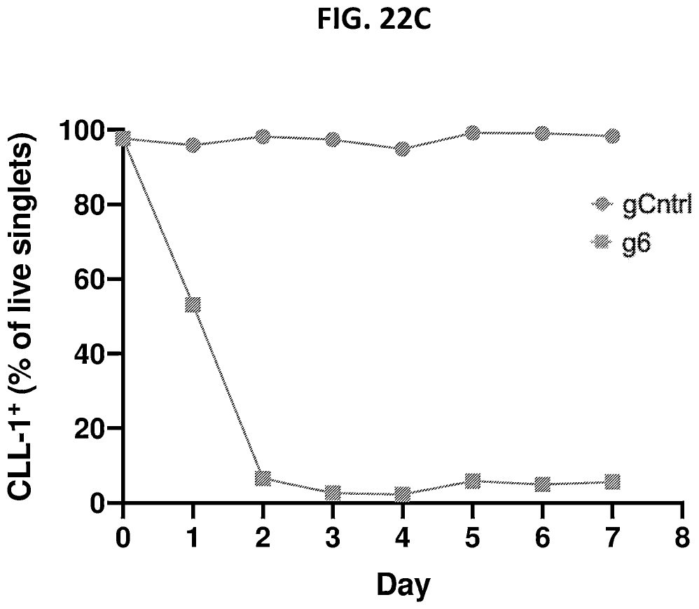

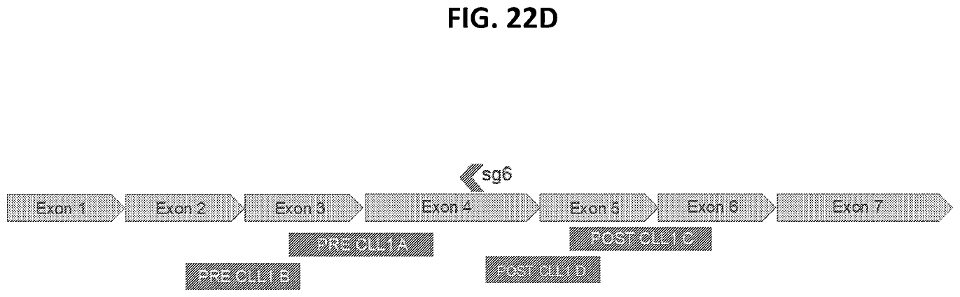

Following multiplexed editing, cells were analyzed for expression kinetics and protein stability of CD33 and CLL-1. FIG. 21A and FIG. 22A show that highly efficient gene editing of CD33 and CLL-1 editing frequencies were maintained for at least 5 days following multiplex editing. This also resulted in a rapid reduction in CD33 and CLL-1 mRNA levels post-electroporation relative to control cells as early as 1 day post-editing, as shown in FIGS. 21B and 22B, respectively. In addition to reduced transcript levels, edited cells exhibited a significant loss in CD33 and CLL-1 surface protein expression within 2 days post-electroporation relative to cells edited with a control gRNA. FIGS. 21C and 22C. Importantly, the reductions in transcript and protein levels were retained over time.

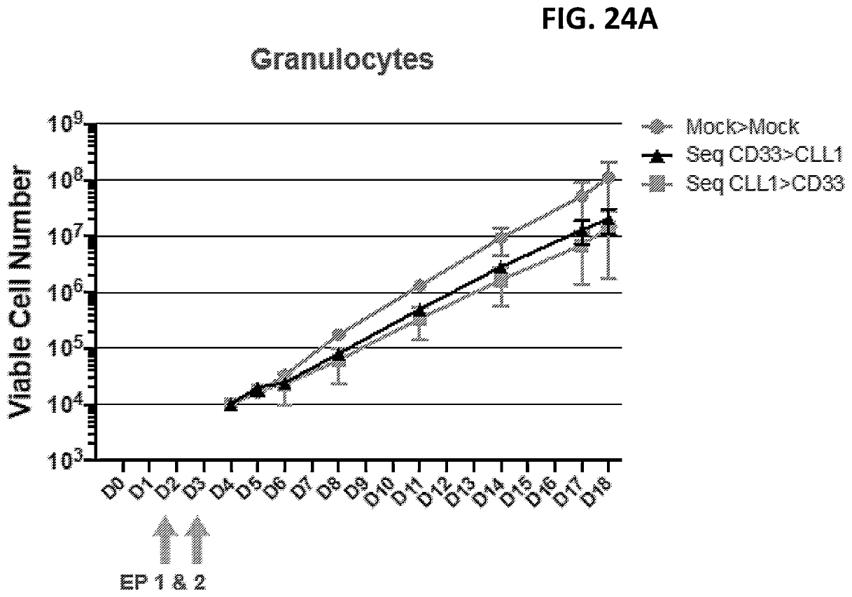

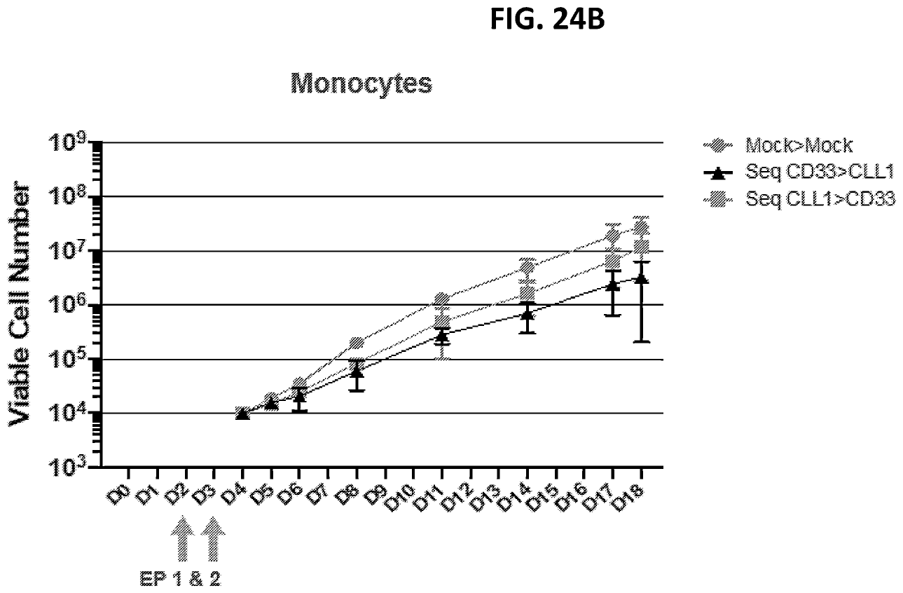

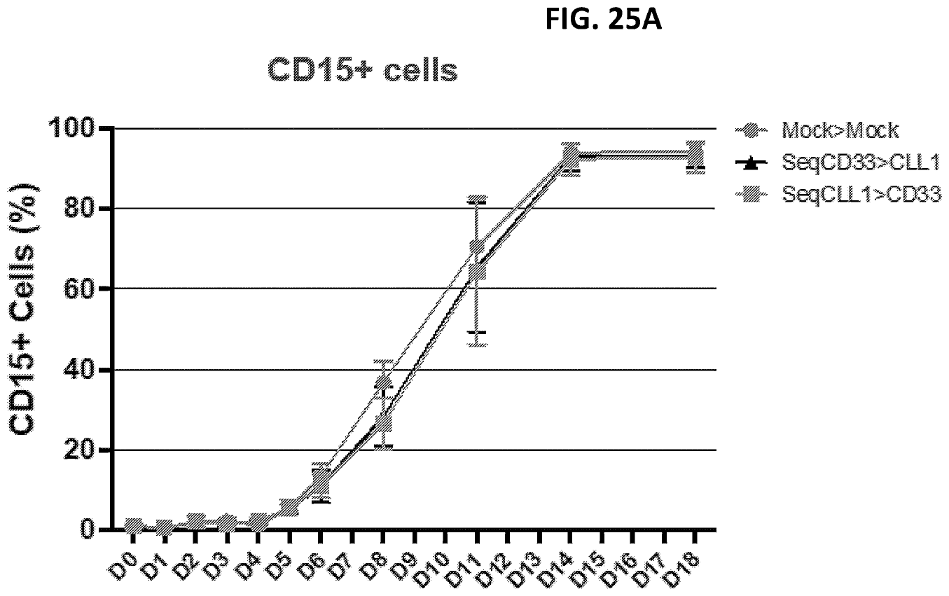

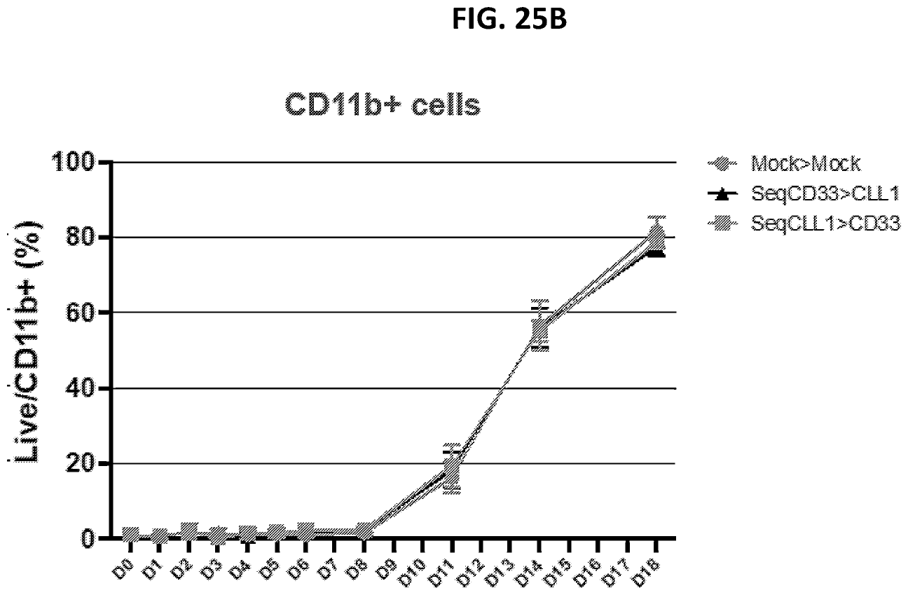

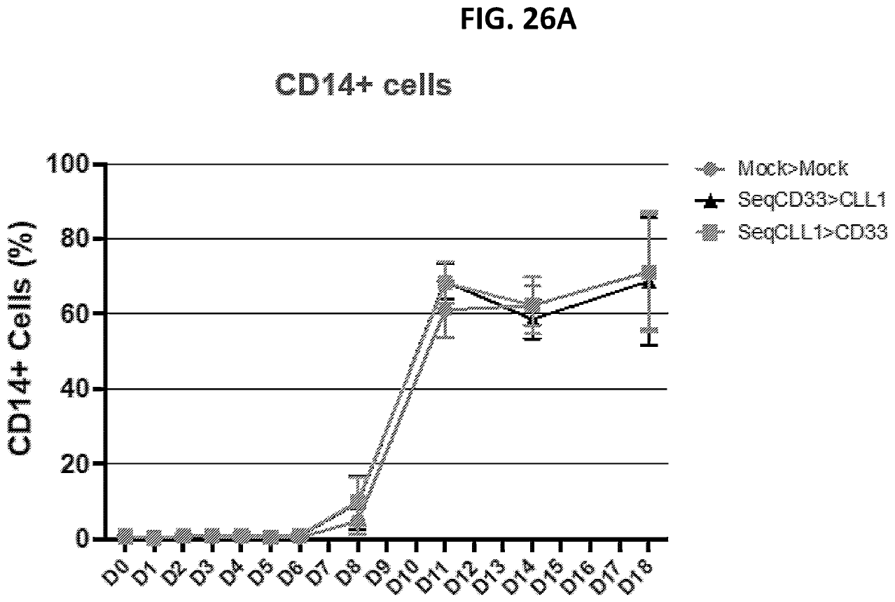

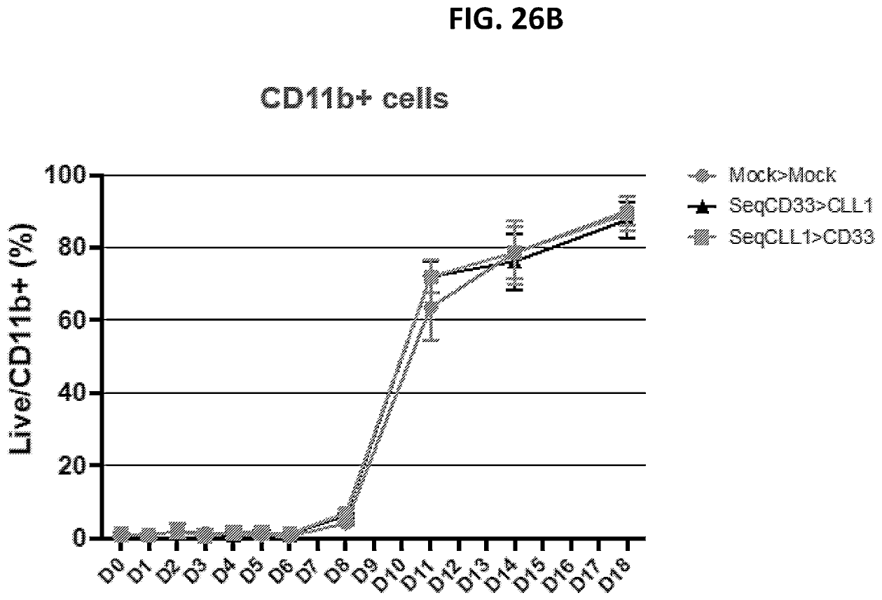

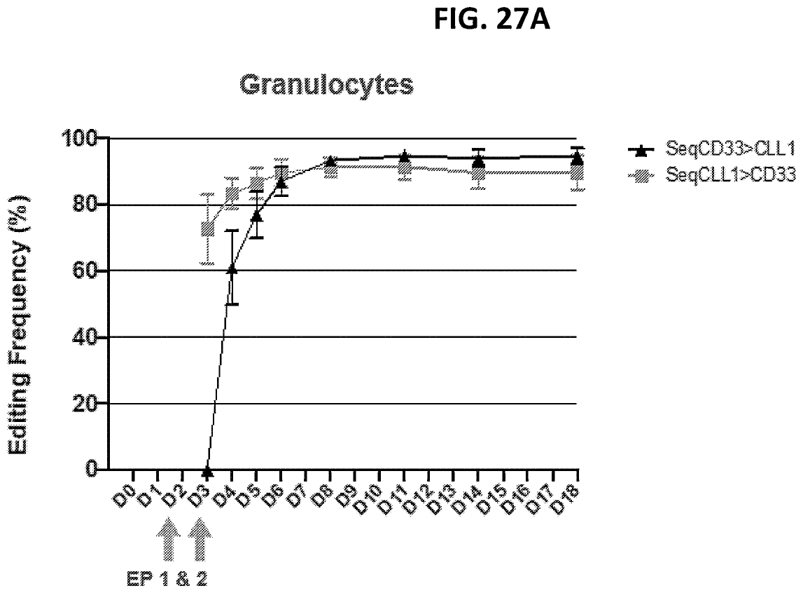

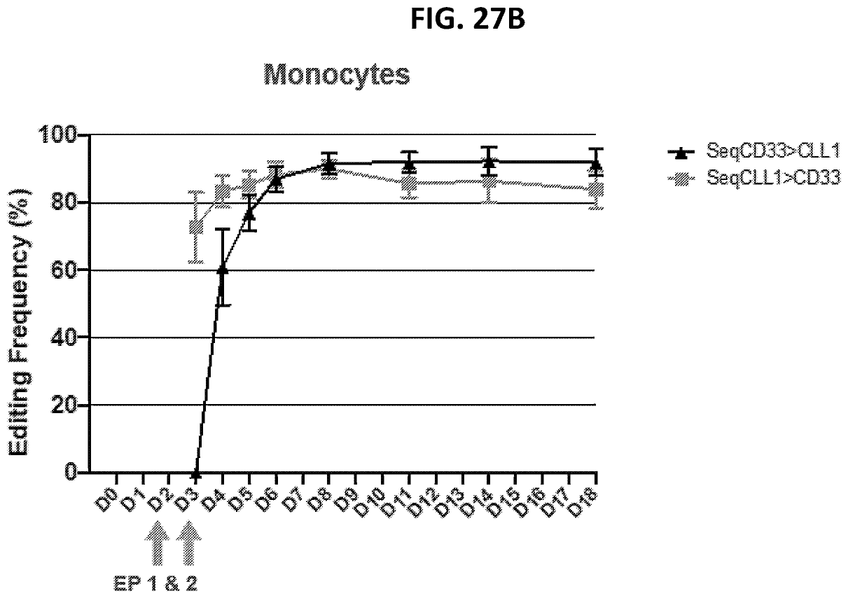

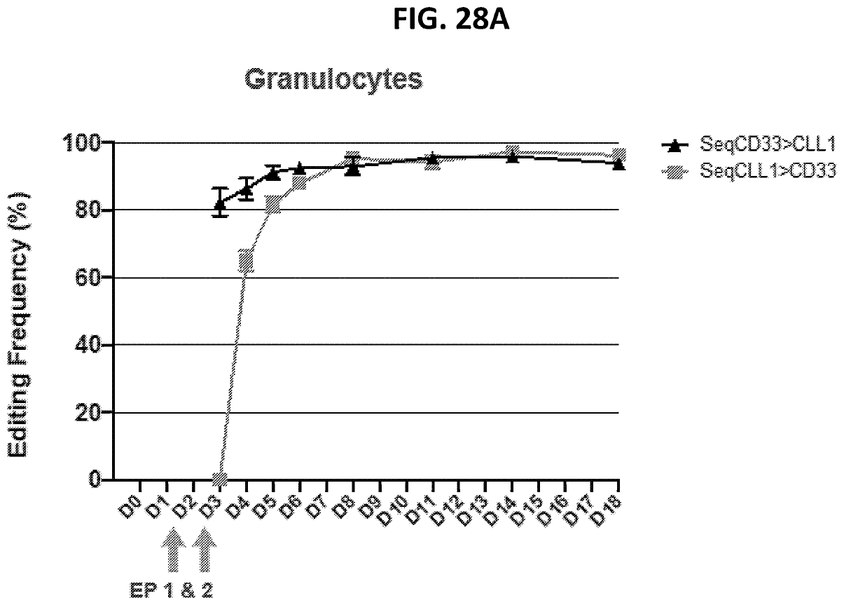

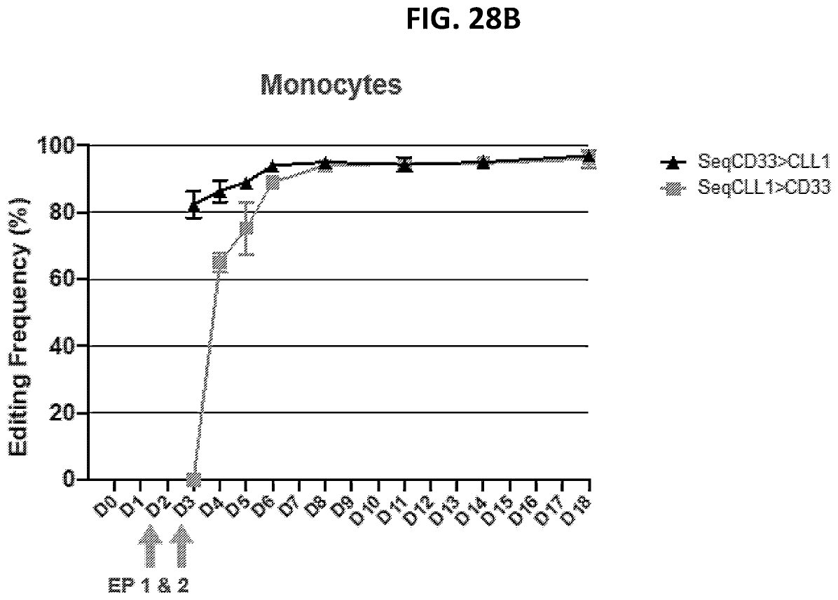

Multiplexed edited cells were characterized for ability to differentiate into functional myeloid cells in in vitro differentiation. FIGS. 24A and 24B show that multiplexed edited cells exhibited comparable growth rates during differentiation into granulocytes and monocytes relative to mock edited control cells, and did not impact granulocyte differentiation (FIGS. 25A and 25B) or monocyte differentiation (FIGS. 26A and 26B) FIG. 27A-28B show that the high level of CLL-1 and CD33 editing achieved was maintained throughout myeloid differentiation.

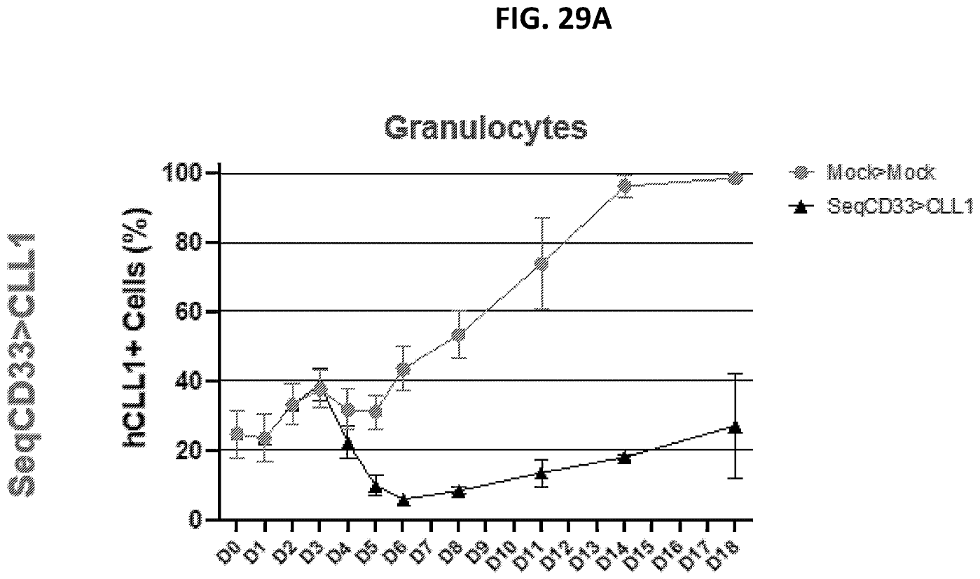

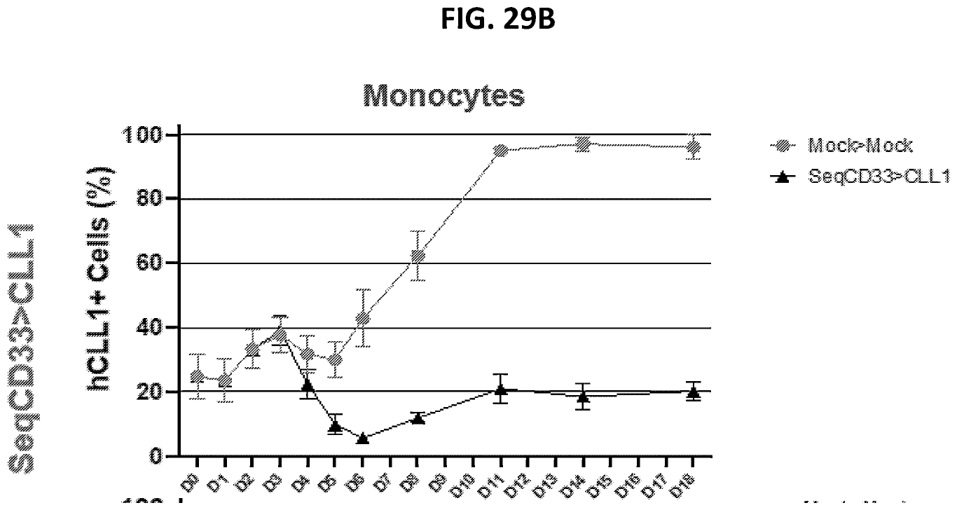

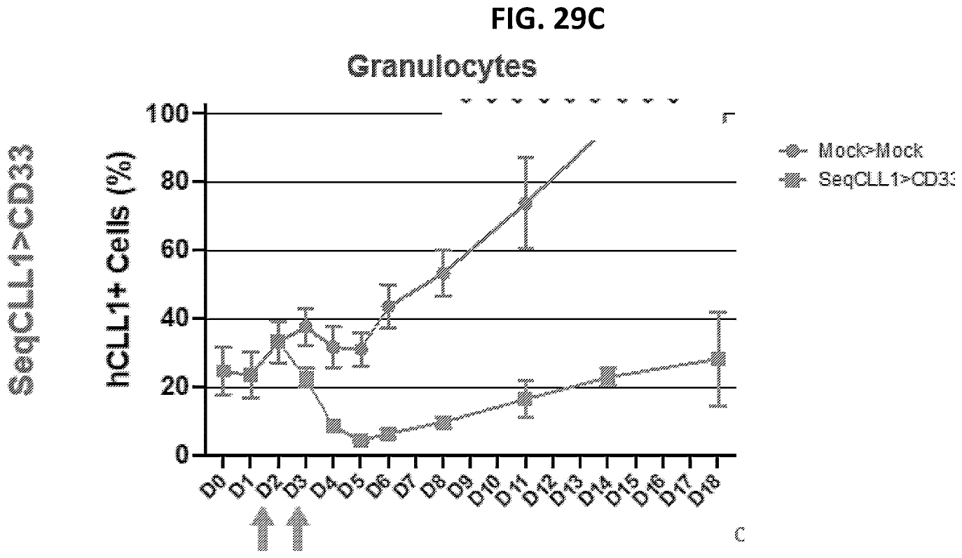

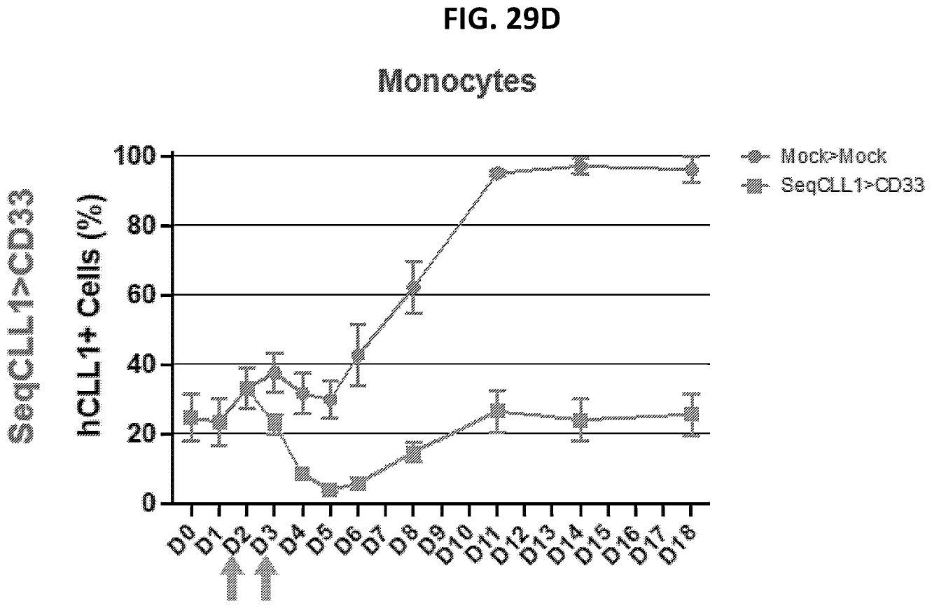

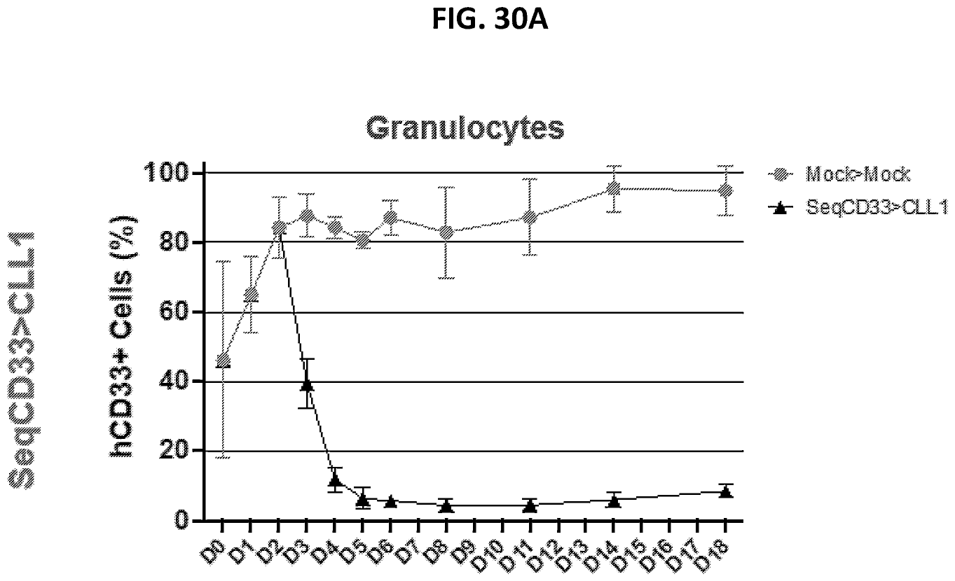

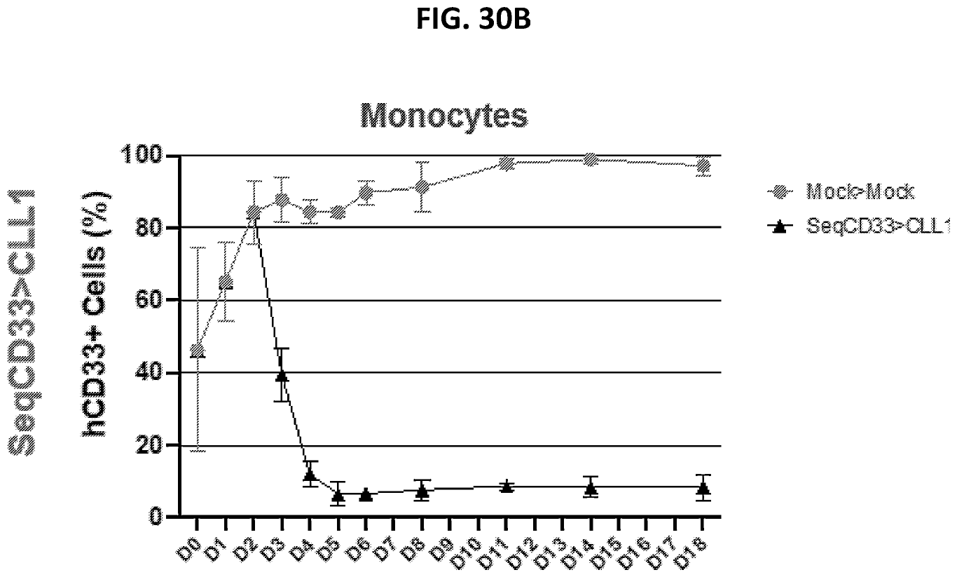

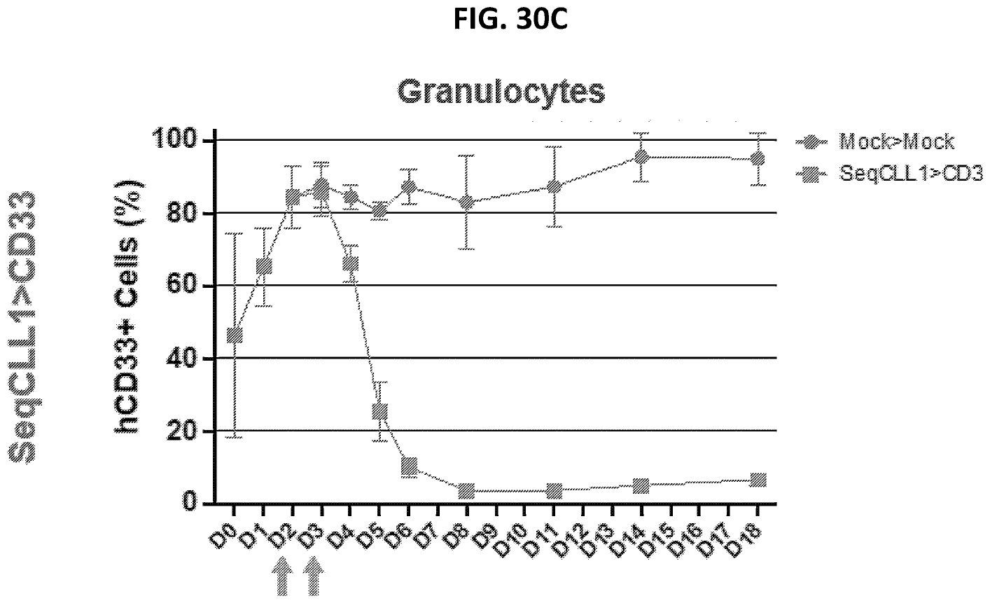

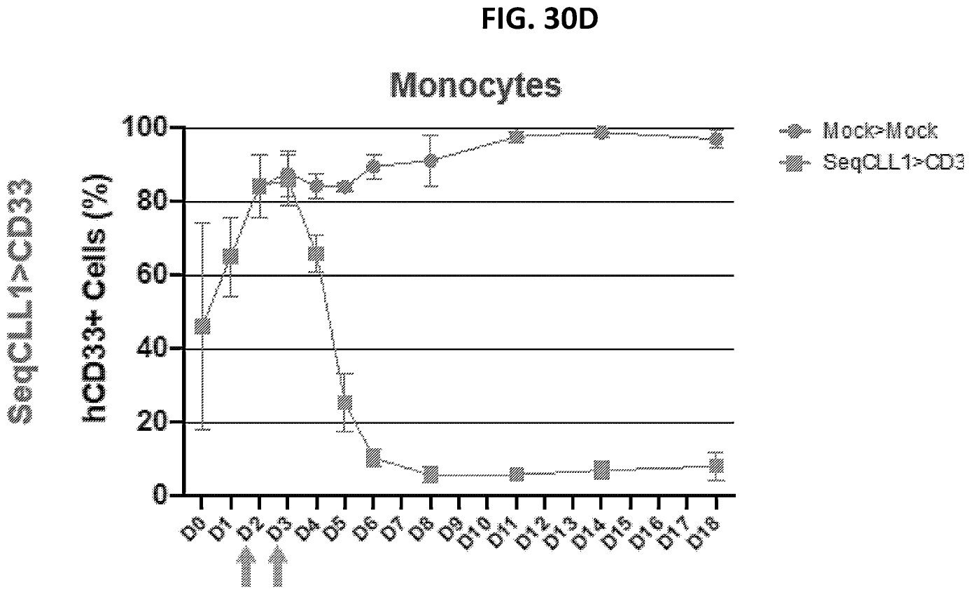

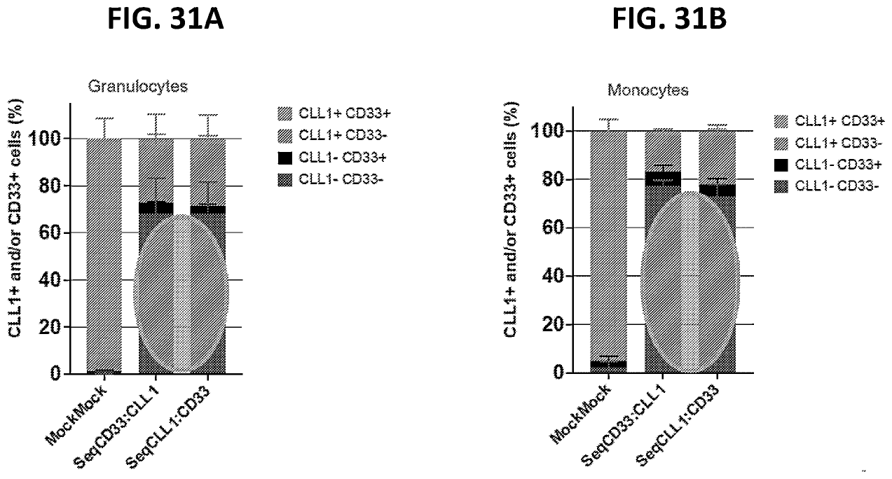

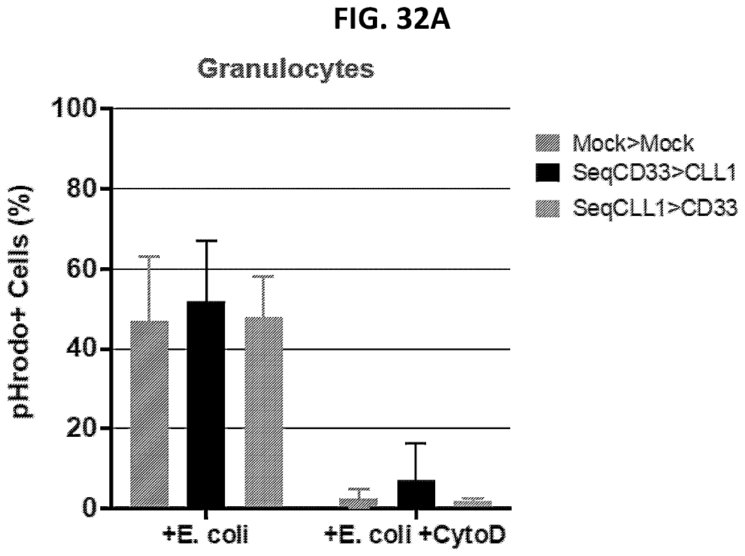

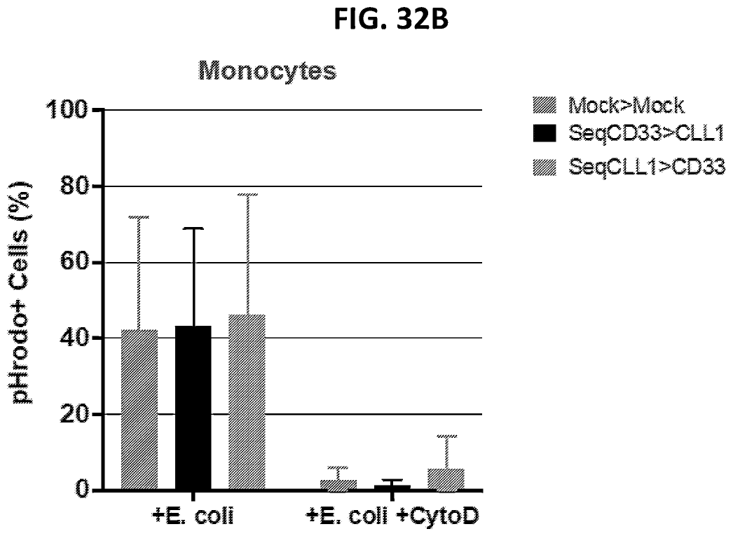

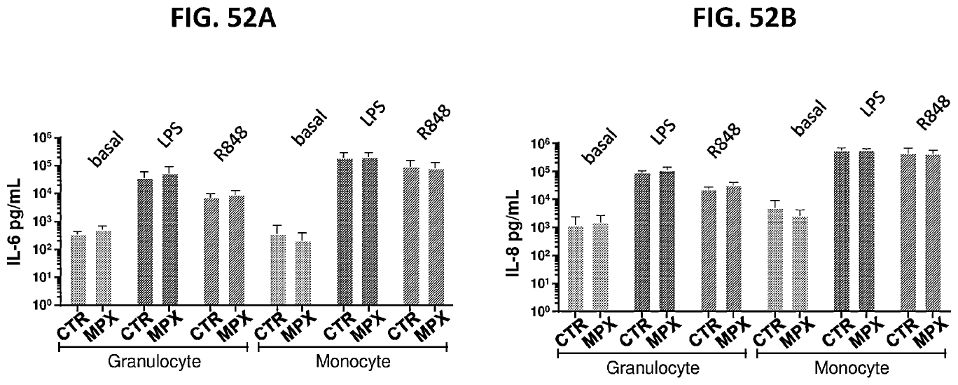

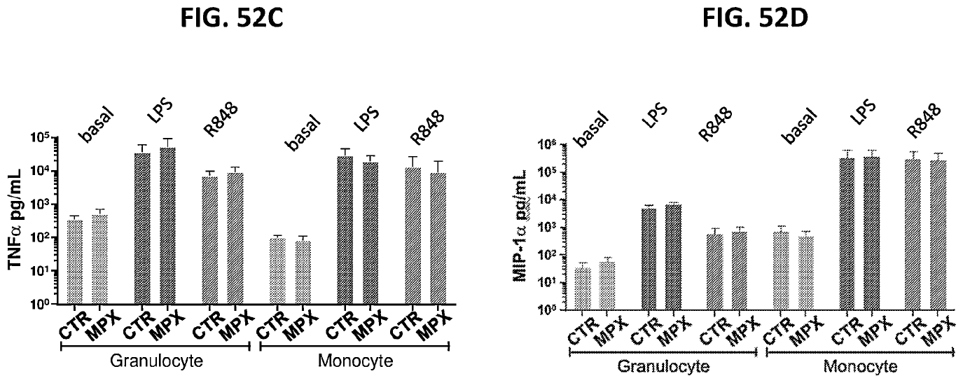

FIGS. 29A-30D show that detectable surface CLL-1 and CD33 protein levels were lost after multiplexed editing and remained significantly reduced throughout the course of myeloid cell differentiation. FIGS. 31A and 31B show that the majority of the population of granulocytes and monocytes comprises cells deficient in both CLL-1 and CD33. FIGS. 32A and 32B show that granulocytes and monocytes differentiated from multiplexed edited human HSCs retained phagocytic activity, demonstrating comparable E. coli phagocytosis levels to mock edited cells. FIGS. 52A-52D show differentiated cells derived from CD33 and CLL-1 multiplex edited hHSCPs maintained function comparable to control cells, as assessed by cytokine production following stimulation with LPS or R848.

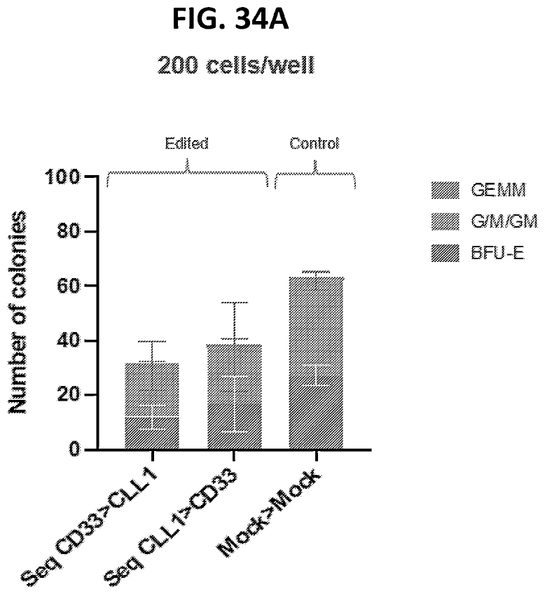

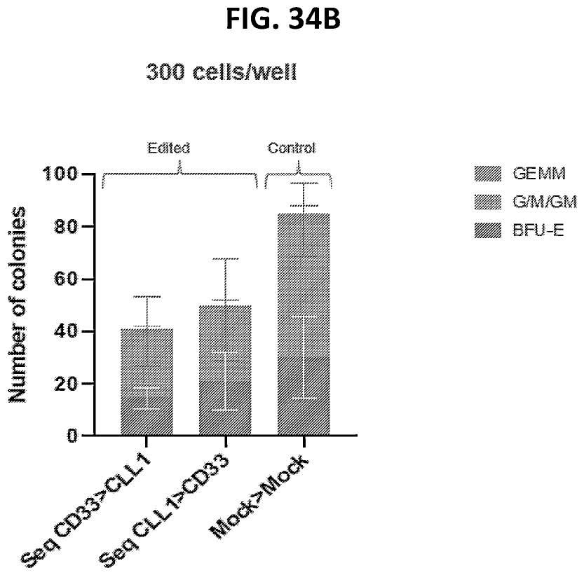

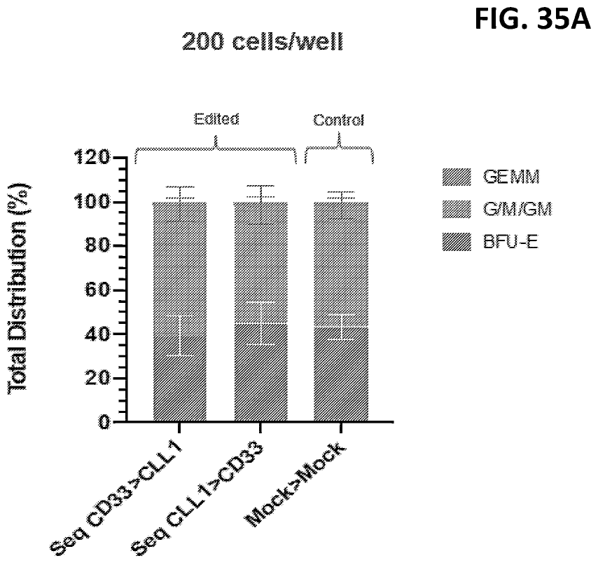

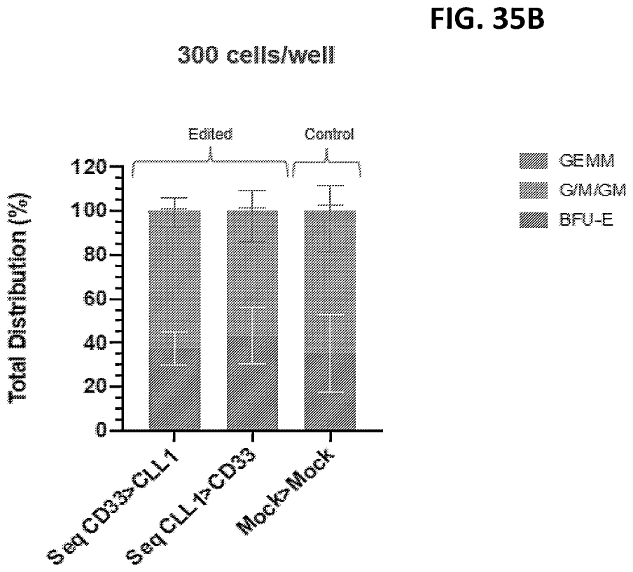

FIGS. 34A-35B show that multiplexed edited cells were capable of forming colony units comprising, erythroid cells, granulocytes, macrophages, and megakaryocytes.

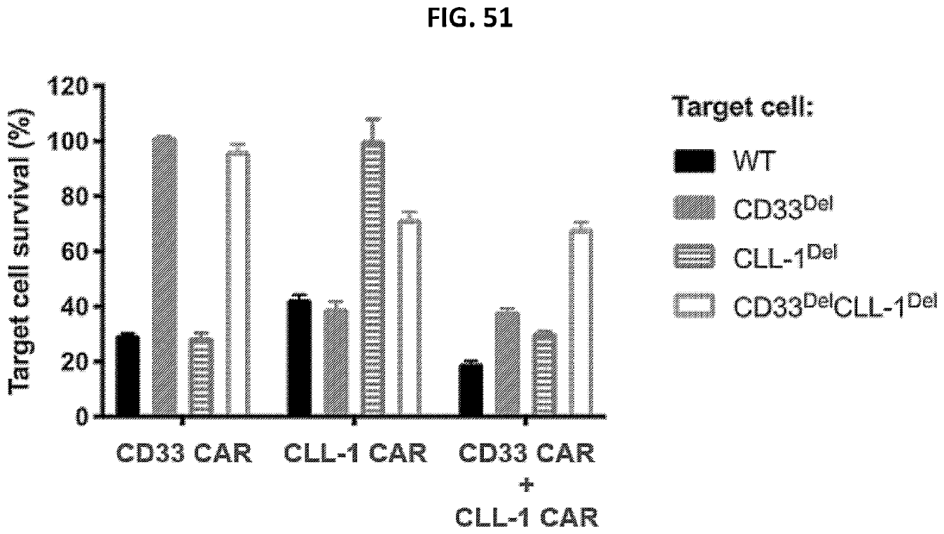

Multiplexed edited cells were also assessed for survival to CD33- and CLL-1-targeted therapies, such as cells expressing chimeric antigen receptors (CARs) targeting CD33 or CLL-1. FIG. 51 shows that CARs targeting CD33 or CLL-1 induced cytotoxicity of cells expressing the wild-type antigens (e.g., CD33, CLL-1), however cells lacking CD33 or CLL-1 were resistant to the cognate CAR expressing cells. Multiplex edited cells lacking CD33 and CLL-1 showed increased survival to both CD33 targeting CARs and CLL-1 targeting CARs. These results indicate that multiplex editing of CD33 and CLL-1 rendered the cells resistant to therapies targeting CD33 and CLL-1.

### Example 6: In Vivo Engraftment of Multiplex Edited Cells Into Mouse Models

This example demonstrates that multiplex edited human HSCs can be engrafted long-term into mouse models. In particular, this example demonstrates that engrafted multiplex edited cells stably repopulate blood and bone marrow tissues of the engrafted mice. This example also demonstrates that engraftment of multiplex edited cells did not significantly impact myeloid and lymphoid differentiation.

### Methods

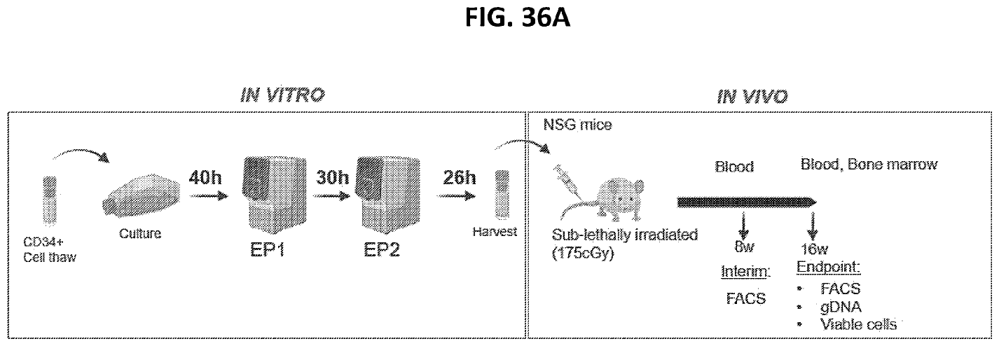

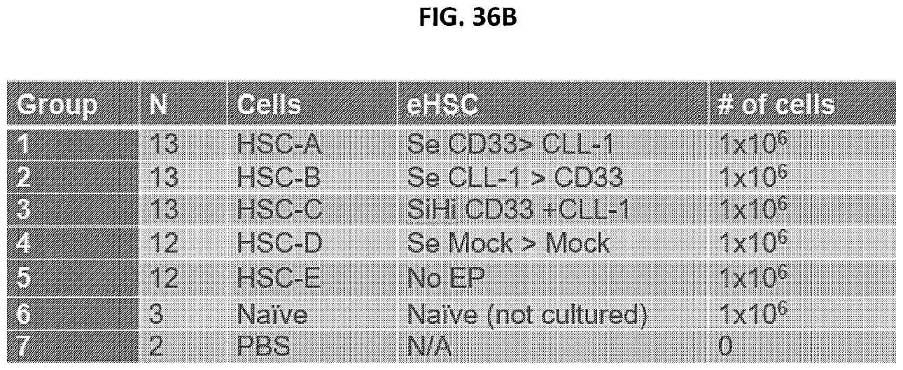

As shown in FIG. 36A, cells were thawed and sequentially electroporated as described above. Twenty-six hours post-electroporation 2 (EP2), cells were harvested and injected into NSG™ mice that were sub-lethally irradiated at a dose of 175 cGY. Blood samples were obtained for interim analysis at 8 weeks post-engraftment, and blood and bone marrow samples were obtained for analysis at 16 weeks post-engraftment. FIG. 36B shows treatment groups for the engraftment study.

### Results

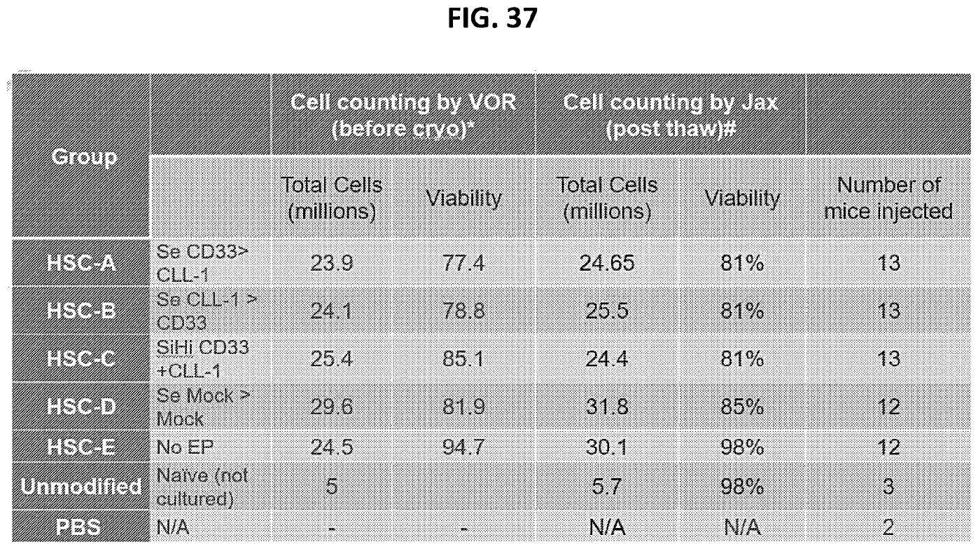

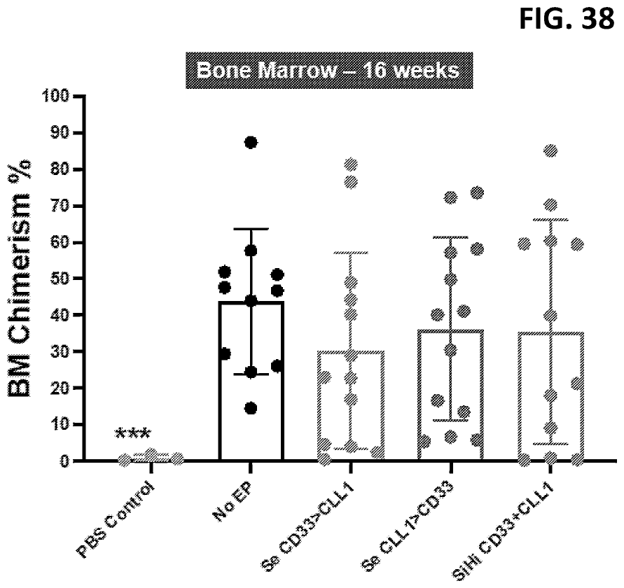

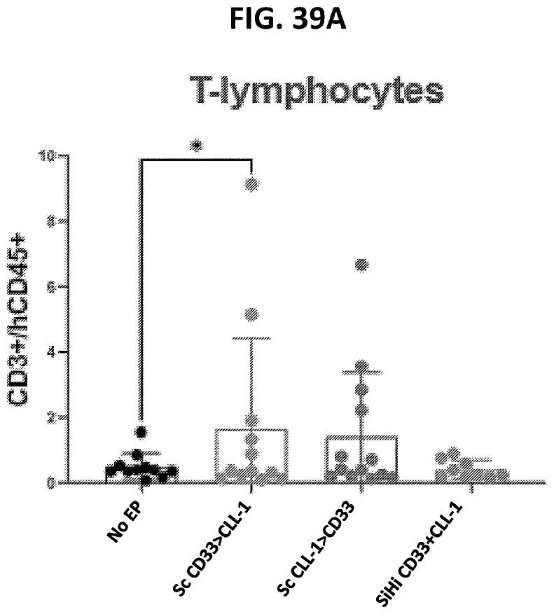

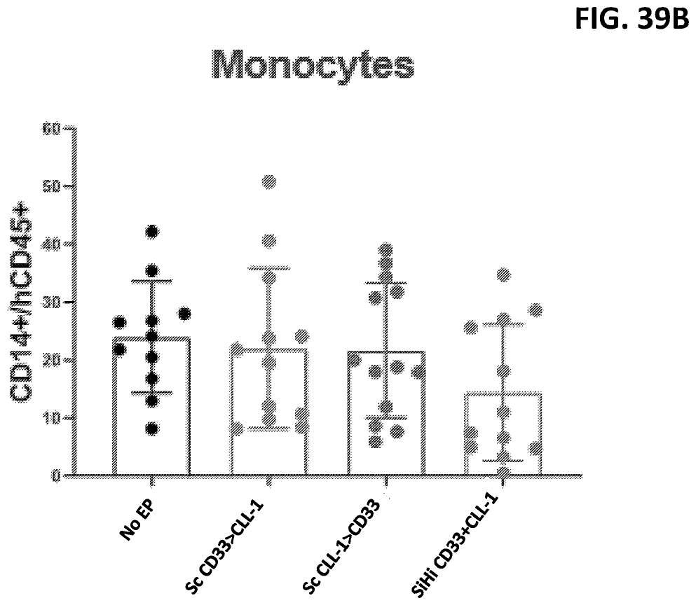

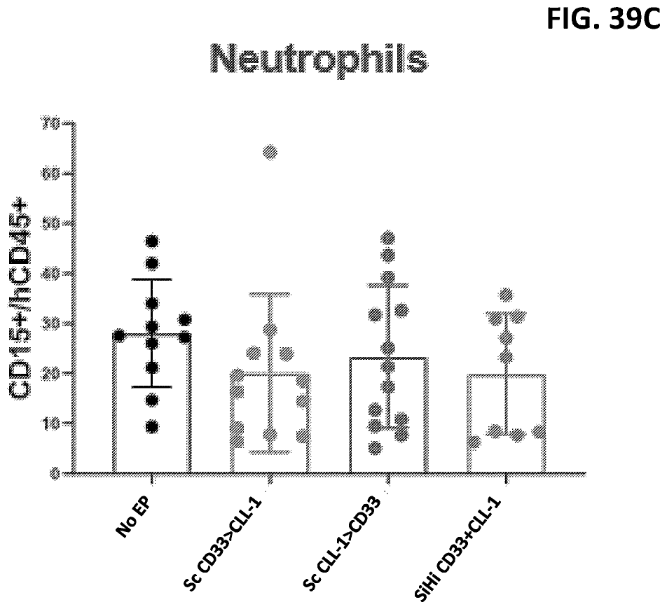

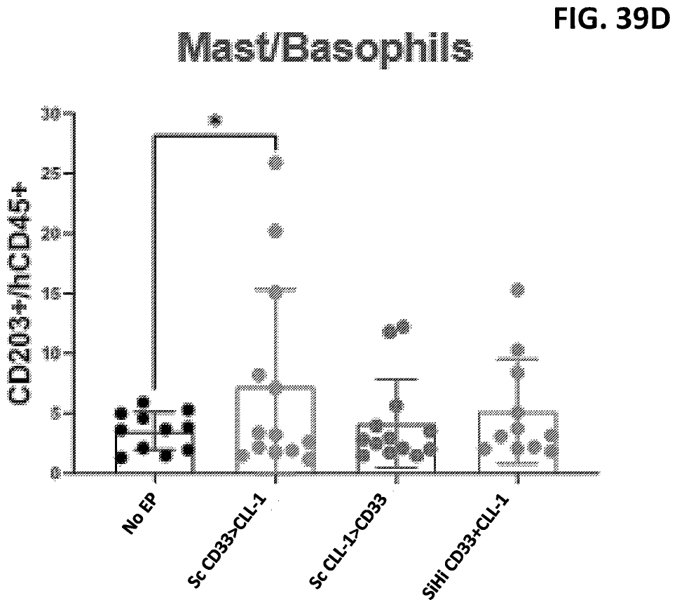

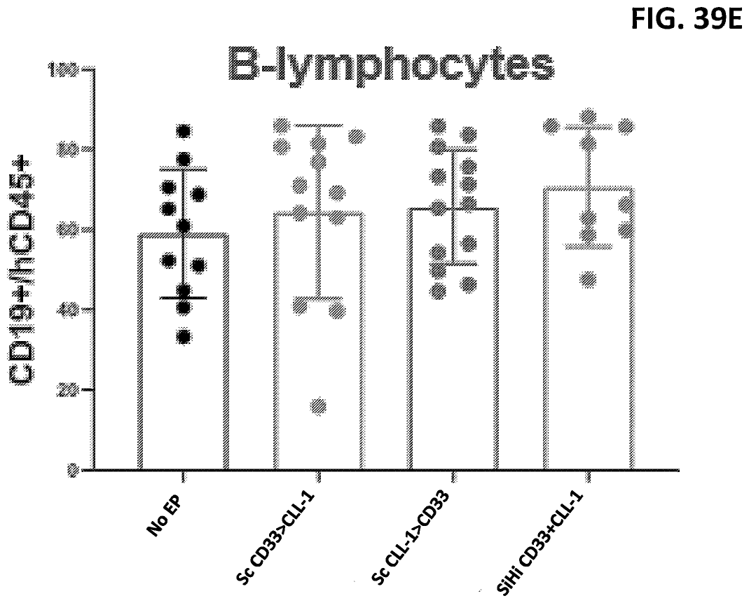

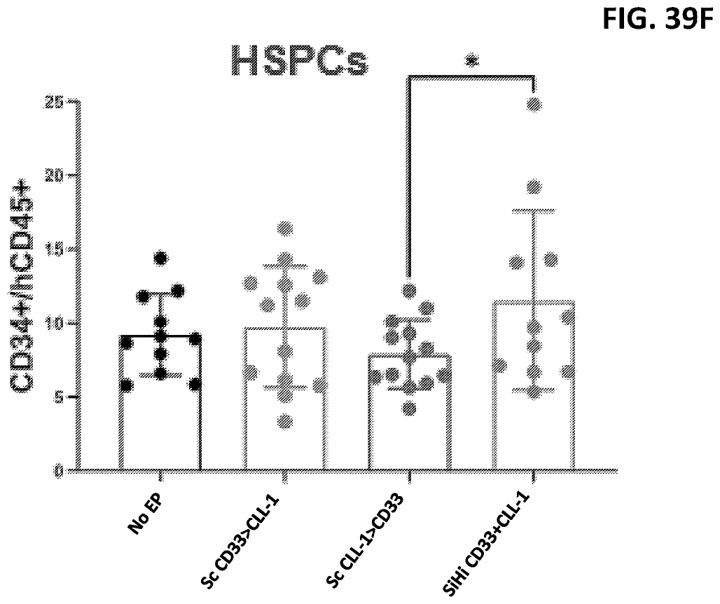

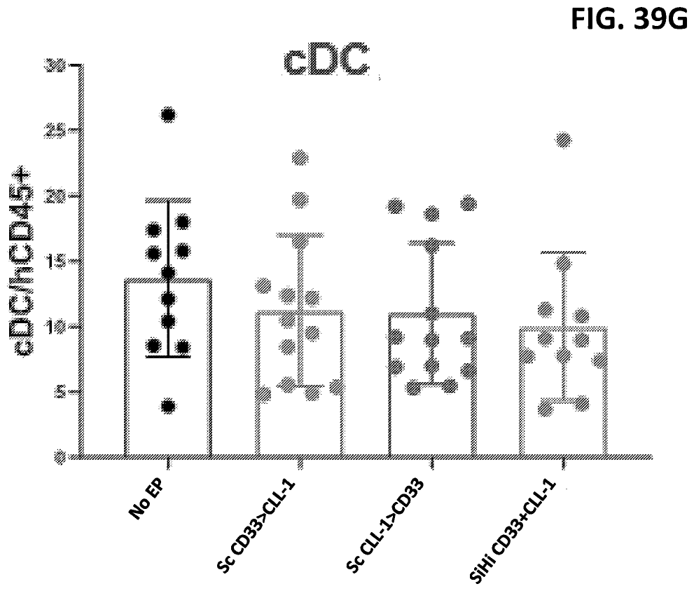

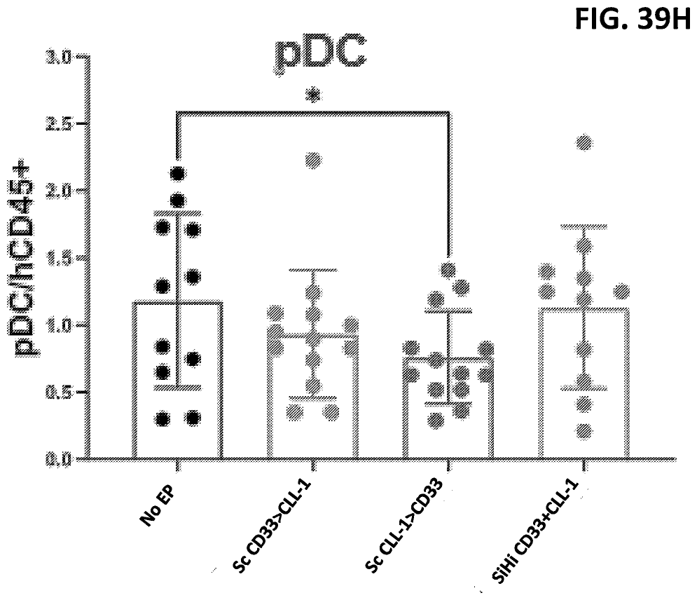

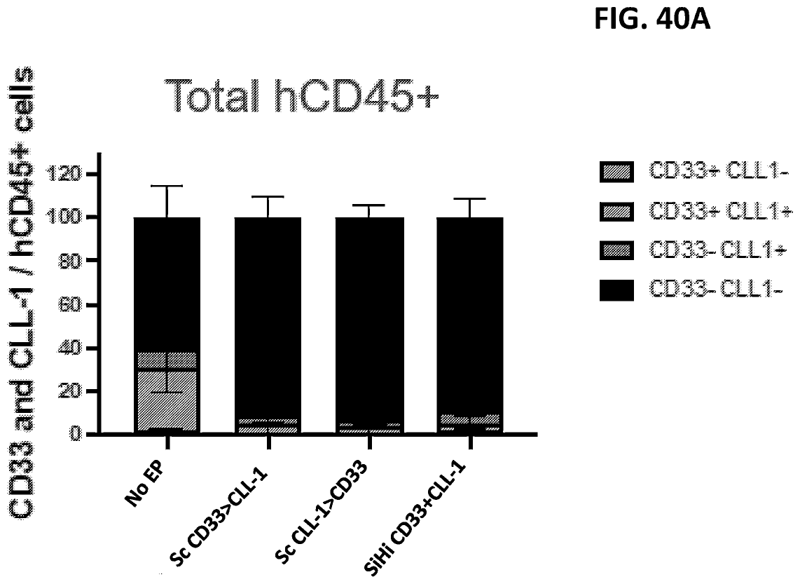

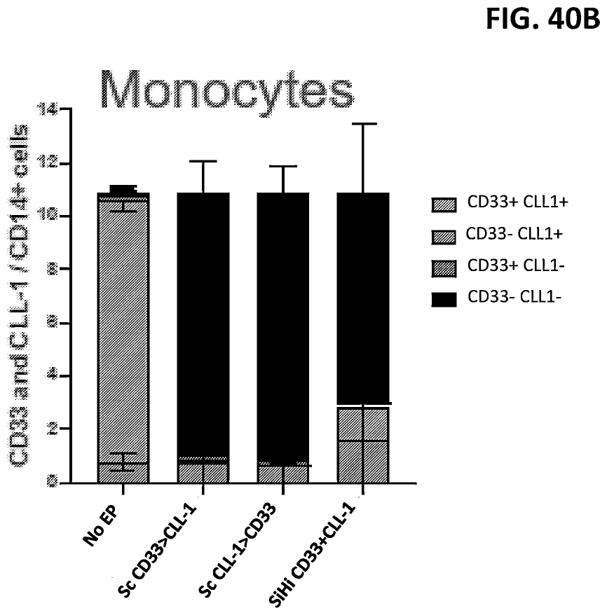

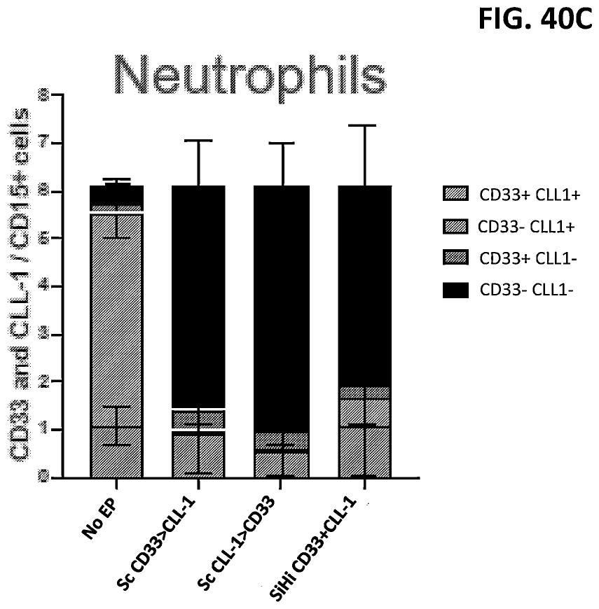

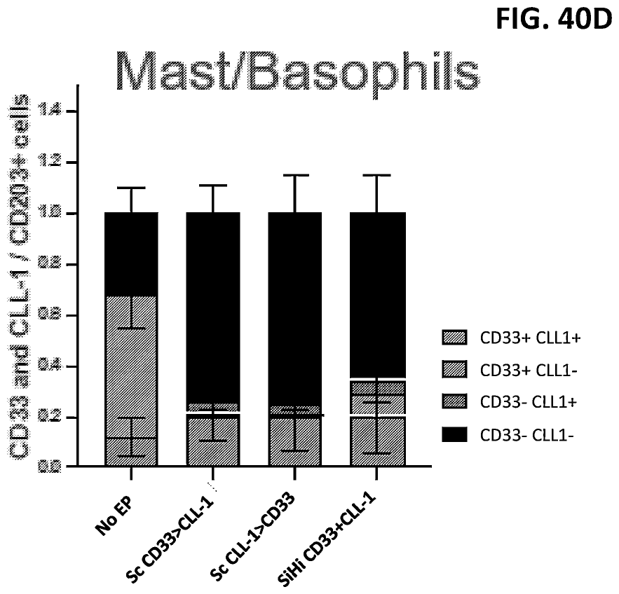

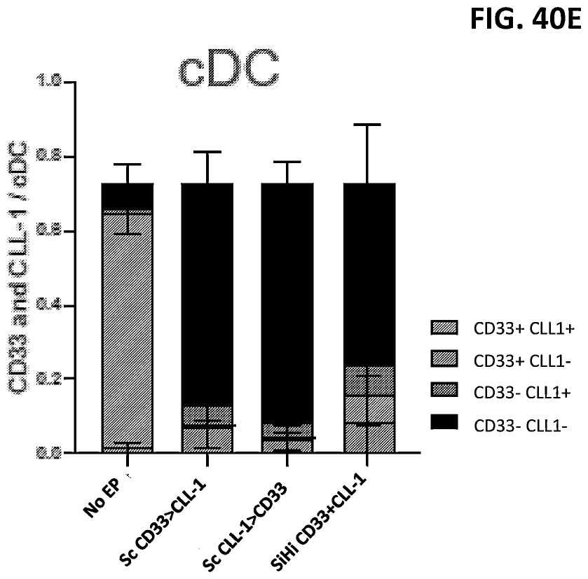

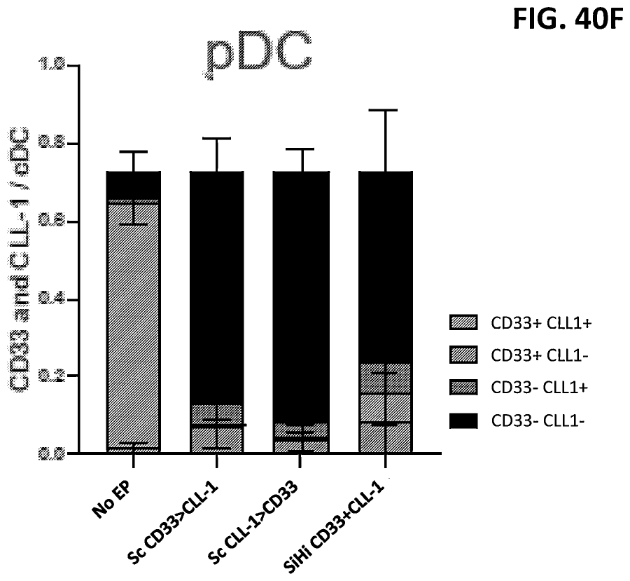

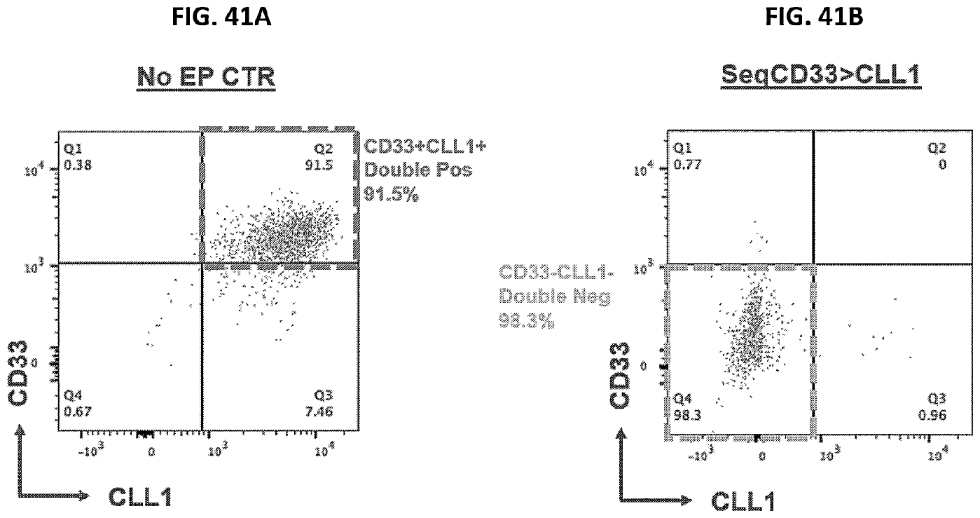

FIG. 37 shows cell counts and viability analyses of cells following prior to cryopreservation or post-thawing. FIG. 38 shows analysis of bone marrow chimerism in engrafted mice, indicating a similar level of bone marrow chimerism in the multiplex edited groups as compared to the no electroporation (No EP) control group. FIG. 39A-39H show that multiplex editing did not significantly impact myeloid and lymphoid lineages compared to the no electroporation control group. It was also observed that the multiplexed editing was highly effective even in cells that express high levels of CD33 and CLL-1 (e.g., monocytes, mast cells, basophils, cDCs, and pDCs). See, FIG. 40A-40F. Reduced expression of both CD33 and CLL-1 was also persistent at least to the 16-week post engraftment time point. FIGS. 41A and 41B.

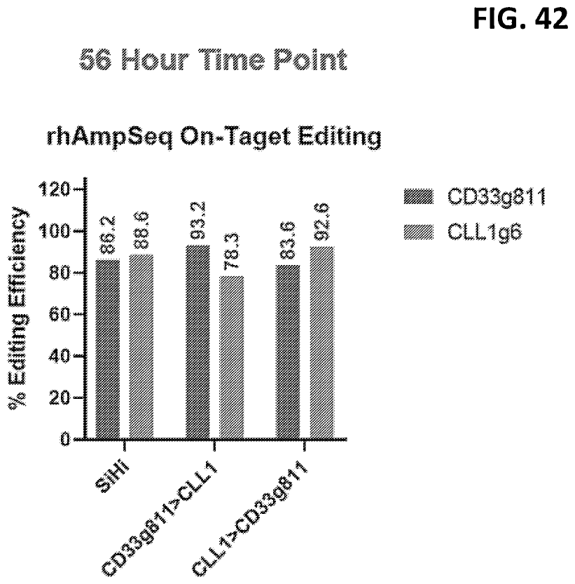

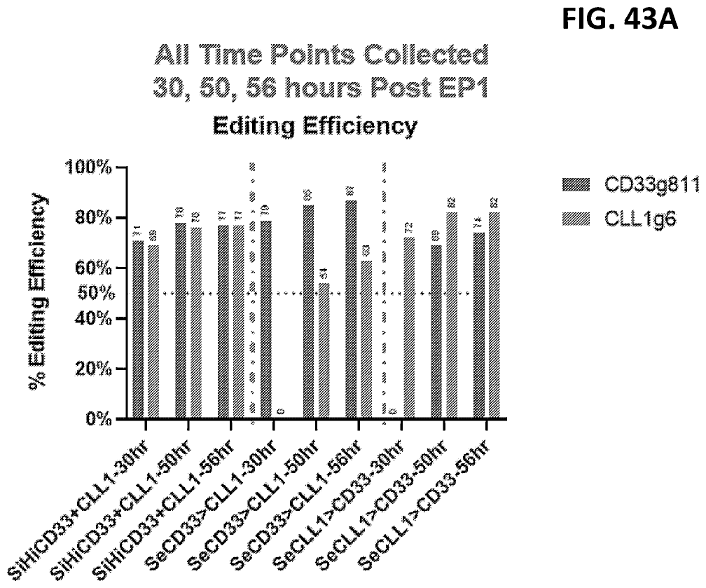

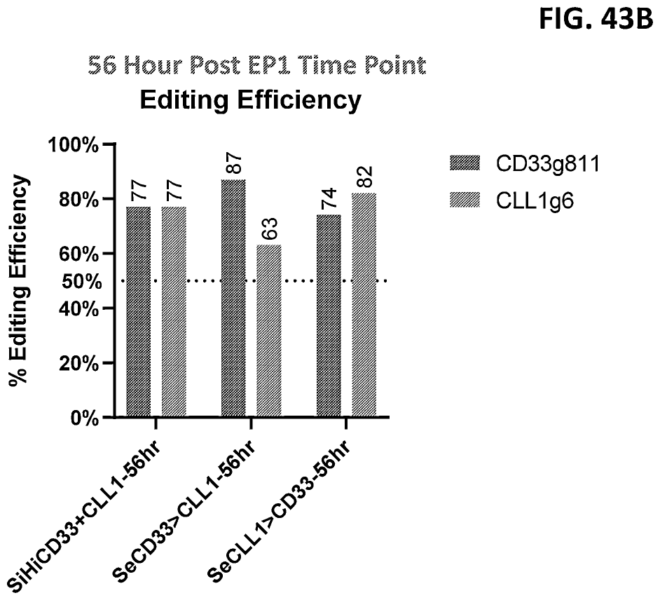

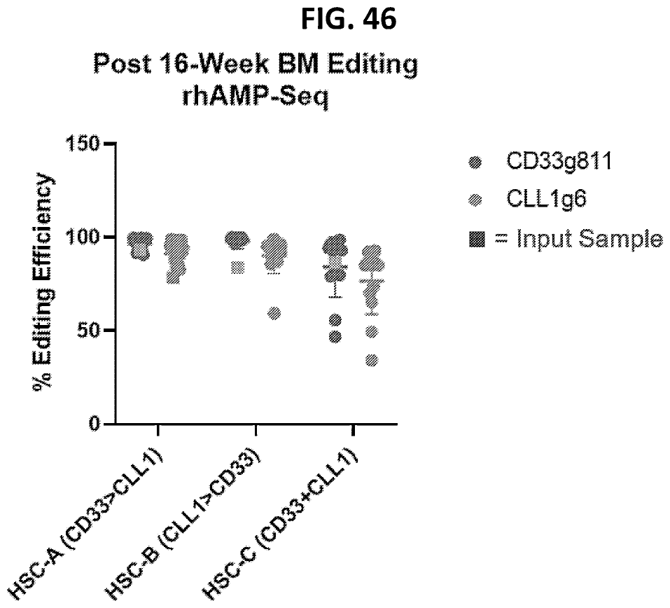

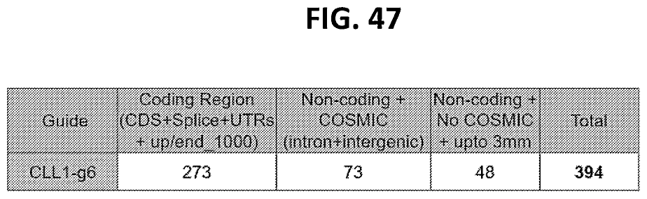

Multiplex edited cells were further characterized by sequencing analyses following engraftment to assess the in vivo persistence of editing and any translocation events. As shown in FIG. 42, on-target editing analysis using rhAmpSeq demonstrated that a high level of editing was achieved across all treatment groups at the 56-hour time point following EP1. Further analysis at additional time points (e.g., 30 hours, 50 hours, 56 hours post-EP1) using ICE analysis. FIGS. 43A and 43B. ICE analysis confirmed the high level of editing efficiency rhAMP-Seq analysis of multiplex edited cells also revealed high levels of editing efficiency of human HSCs that were harvested from engrafted mouse models at 16 weeks post-engraftment. See FIG. 46.

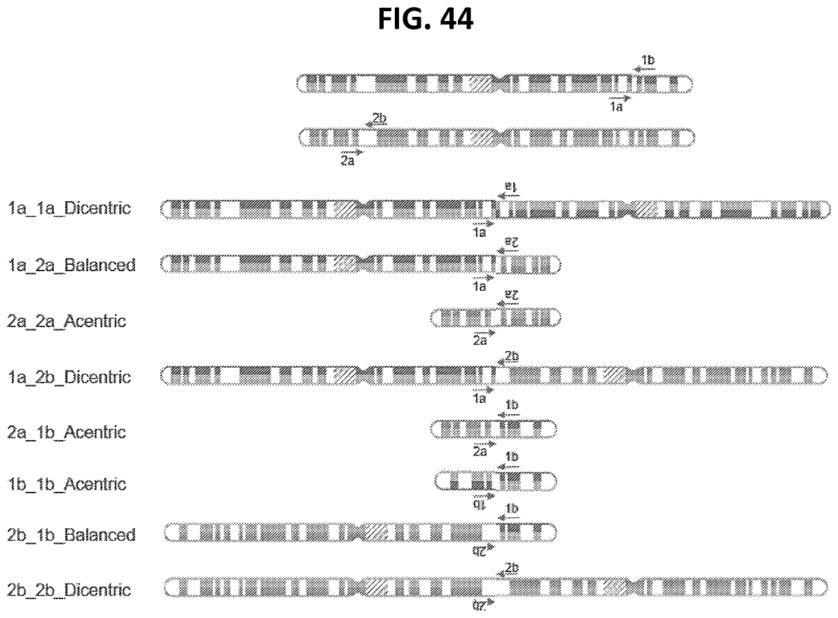

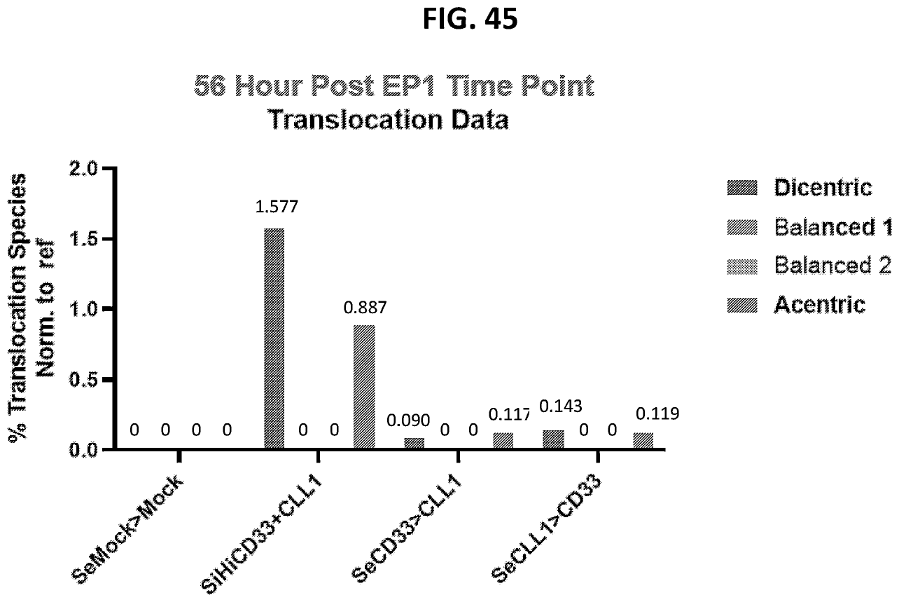

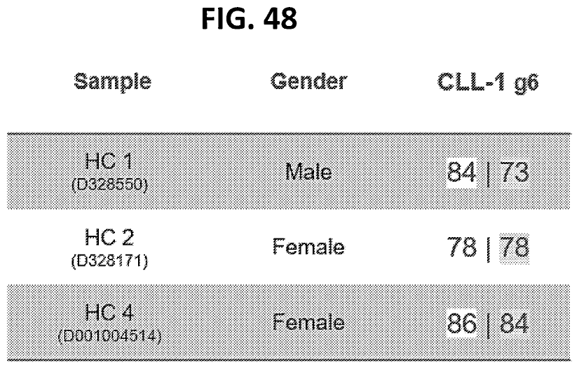

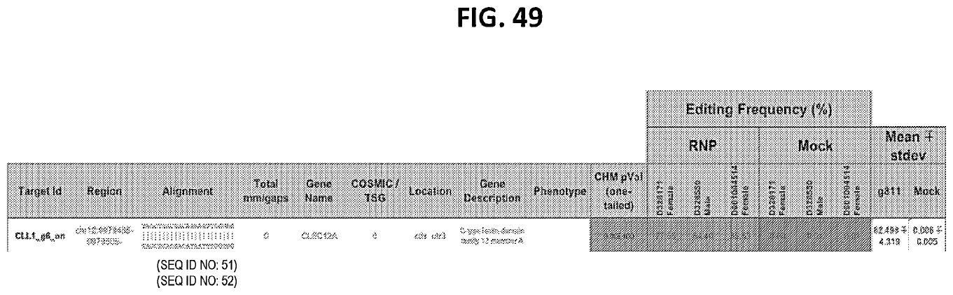

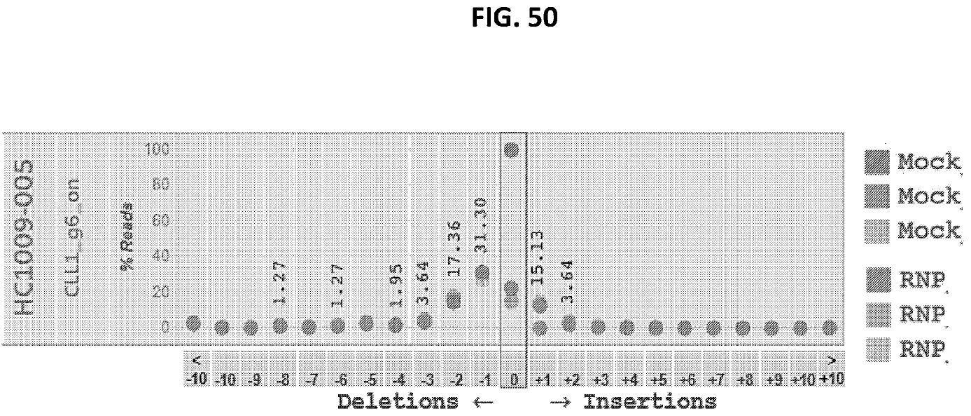

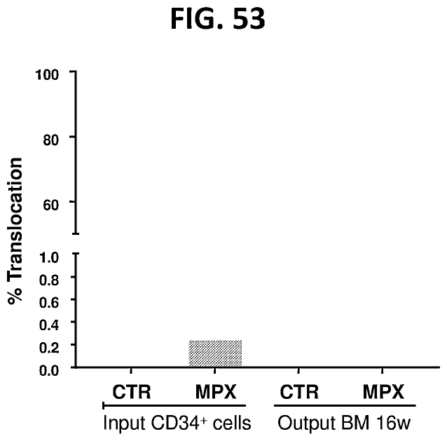

Chromosomal translocation events may arise as a result of multiplex editing. See, FIG. 44. However, as shown in FIGS. 45 and 53, a very low frequency of translocation events were detected in the input samples following sequential electroporation. Additionally, analysis of cells harvested from the mice 16-weeks post engraftment indicated a comparable level of editing efficiency in the “output” bone marrow samples as compared to the “input” sequentially edited samples with a low level of off-target editing events. See FIGS. 46 and 48-51. Collectively, these data indicate that high levels of double deletion engrafted humans HSCs persist after engraftment in a manner which coincides with no major impact in lymphoid and myeloid cell reconstitution.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A method comprising:
a) contacting a plurality of cells with (i) a first gRNA comprising a first targeting domain that binds to a first target sequence and (ii) an RNA guided-nuclease that binds the first gRNA, thus forming a first ribonucleoprotein (RNP) complex under conditions suitable for the first gRNA to form and/or maintain a first RNP complex with the RNA-guided nuclease of (ii) and for the first RNP complex to bind the first target sequence; and
b) contacting the plurality of cells with (iii) a second gRNA comprising a second targeting domain that binds a second target sequence and (iv) an RNA guided nuclease that binds the second gRNA to form and/or maintain a second RNP complex with the RNA guided nuclease of (iv) and for the second RNP complex to bind the second target sequence;
thereby producing a population of genetically engineered cells comprising a genetic modification of the first target sequence and a genetic modification of the second target sequence;
wherein steps (a) and (b) are performed sequentially and in temporal proximity, separated by a time interval,
wherein the first targeting domain is not identical to the second targeting domain.

2. The method of claim 1, wherein the genetic modification of the first target sequence consists of an insertion or deletion at or immediately proximal to a site cut by the RNA-guided nuclease when bound to the first gRNA; and/or the genetic modification of the second target sequence consists of an insertion or deletion immediately proximal to a site cut by the RNA-guided nuclease when bound to the second gRNA.

3. The method of claim 1 or 2, wherein the method produces a population of translocation product cells, wherein each cell of the subpopulation comprises a translocation product comprising a portion of the genome comprising the first target sequence, a portion of the genome comprising the second target sequence, or both.

4. The method of claim 3, wherein the method produces fewer translocation product cells as compared to a method comprising contacting a plurality of cells with the second gRNA of (iii) prior to contacting the plurality of cells with the first gRNA of (i).

5. The method of claim 3 or 4, wherein the method produces at least 10% fewer translocation product cells as compared to a method comprising contacting the plurality of cells with the second gRNA of (iii) prior to contacting the plurality of cells with the first gRNA of (i).

6. The method of claim 3, wherein the method produces fewer translocation product cells as compared to a method comprising contacting the plurality of cells with the first gRNA of (i) and the second gRNA of (iii) at substantially the same time.

7. The method of claim 3 or 6, wherein the method produces at least 10% fewer translocation product cells as compared to a method comprising contacting the plurality of cells with the first gRNA of (i) and the second gRNA of (iii) at substantially the same time.

8. The method of any one of claims 1-7, wherein binding of the first RNP complex comprising (i) and (ii) to the first target sequence results in a genetic modification generated by a Non-Homologous End Joining (NHEJ) event.

9. The method of any one of claims 1-8, wherein binding of the RNP complex comprising (i) and (ii) to the first target sequence produces a fast-resolving double strand break.

10. The method of any one of claims 1-9, wherein binding of the second RNP complex comprising (iii) and (iv) to the second target sequence results in a genetic modification generated by a microhomology-mediated end joining (MMEJ) event.

11. The method of any one of claims 1-10, wherein binding of the second RNP complex comprising (iii) and (iv) to the second target sequence produces a slow-resolving double strand break.

12. The method of any one of claims 1-11, wherein the first target sequence is present in a first gene, a transcriptional control element operably linked thereto, or a portion of the gene and transcriptional control element.

13. The method of claim 12, wherein the genomic modification of the first target sequence results in reduced or eliminated expression of the product encoded by the first gene, or expression of a variant of the product expressed by wild-type cells of the same cell type that do not harbor a genomic modification in the first target sequence.

14. The method of claim 12 or 13, wherein the first gene encodes a first lineage-specific cell-surface antigen.

15. The method of claim 14, wherein the first lineage-specific cell-surface antigen is selected from the group consisting of CD33, CD19, CD123, CLL-1, CD30, CD5, CD6, CD7, CD38, and BCMA.

16. The method of any one of claims 1-15, wherein the second target sequence is present in a second gene, a transcriptional control element operably linked thereto, or a portion of the gene and transcriptional control element.

17. The method of claim 16, wherein the genomic modification of the second target sequence results in reduced or eliminated expression of the product encoded by the second gene, or expression of a variant of the product expressed by wild-type cells of the same cell type that do not harbor a genomic modification in the second target sequence.

18. The method of claim 16 or 17, wherein the second gene encodes a second lineage-specific cell-surface antigen.

19. The method of claim 18, wherein the second lineage-specific cell-surface antigen is selected from the group consisting of CD33, CD19, CD123, CLL-1, CD30, CD5, CD6, CD7, CD38, and BCMA.

20. The method of any one of claims 15-19, wherein the first lineage-specific cell-surface antigen is CD33.

21. The method of claim 20, wherein the second lineage-specific cell-surface antigen is CD19, CD5, or CLL-1.

22. The method of any one of claims 15-19, wherein the first lineage-specific cell-surface antigen is CD5.

23. The method of claim 22, wherein the second lineage-specific cell-surface antigen is CD33.

24. The method of claim 20 or 21, wherein the first lineage-specific cell-surface antigen is CD33 and the second lineage-specific cell-surface antigen is CLL-1.

25. The method of any one of claims 1-24, wherein the time interval between step (b) and step (c) is at least 4, 6, 8, 10, 12, 14, 16, 18, 20, 22, 24, 26, 28, 30, 32, 34, or 36 hours.

26. The method of any one of claims 1-25, wherein the RNA-guided nuclease of (ii) and/or the RNA-guided nuclease of (iv) is a CRISPR/Cas nuclease.

27. The method of claim 26, wherein the CRISPR/Cas nuclease is a Cas9 nuclease.

28. The method of claim 26, wherein the CRISPR/Cas nuclease is an spCas nuclease.

29. The method of claim 26, wherein the CRISPR/Cas nuclease is an saCas nuclease.

30. The method of claim 26, wherein the CRISPR/Cas nuclease is a Cpf1 nuclease.

31. The method of any one of claims 1-26, wherein the RNA-guided nuclease of (ii) is a Cas9 nuclease and the RNA-guided nuclease of (iv) is a Cpf1 nuclease.

32. The method of any one of claims 1-26, wherein the RNA-guided nuclease of (ii) is a Cpf1 nuclease and the RNA-guided nuclease of (iv) is a Cas9 nuclease.

33. The method of any one of claims 1-26, wherein the RNA-guided nuclease of (ii) and the RNA-guided nuclease of (iv) are Cpf1 nucleases.

34. The method of any one of claims 1-26, wherein the RNA-guided nuclease of (ii) and the RNA-guided nuclease of (iv) are Cas9 nucleases.

35. The method of any one of claims 1-34, wherein the contacting of (a) comprises introducing (i) and (ii) into the cell in the form of a pre-formed ribonucleoprotein (RNP) complex; and/or wherein the contacting of (b) comprises introducing (iii) and (iv) into the cell in the form of a pre-formed ribonucleoprotein (RNP) complex.

36. The method of claim 35, wherein the pre-formed ribonucleoprotein (RNP) complex is introduced into the cell via electroporation.

37. The method of any one of claims 1-36, wherein the contacting of (a) comprises introducing (i) and/or (ii) into the cell in the form of a nucleic acid encoding the gRNA of (i) and/or the RNA-guided nuclease of (ii); and/or
wherein the contacting of (b) comprises introducing (iii) and/or (iv) into the cell in the form of a nucleic acid encoding the gRNA of (i) and/or the RNA-guided nuclease of (ii).

38. The method of any one of claims 1-37, wherein the nucleic acid encoding the first gRNA of (i) and/or the RNA-guided nuclease of (ii) is an RNA, preferably an mRNA or an mRNA analog.

39. The method of any one of claims 1-37, wherein the nucleic acid encoding the second gRNA of (iii) and/or the RNA-guided nuclease of (iv) is an RNA, preferably an mRNA or an mRNA analog.

40. The method of any one of claims 1-39, wherein the first gRNA and/or the second gRNA comprises one or more nucleotide residues that are chemically modified.

41. The method of any one of claims 1-40, wherein the first and/or the second gRNA comprises one or more nucleotide residues that comprise a 2′O-methyl moiety.

42. The method of any one of claims 1-41, wherein the first and/or the second gRNA comprises one or more nucleotide residues that comprise a phosphorothioate.

43. The method of any one of claims 1-42, wherein the first and/or the second gRNA comprises one or more nucleotide residues that comprise a thioPACE moiety.

44. The method of any one of claims 1-43, wherein the cell is a hematopoietic cell.

45. The method of any one of claims 1-44, wherein the cell is a hematopoietic stem cell.

46. The method of any one of claims 1-45, wherein the cell is a hematopoietic progenitor cell.

47. The method of any one of claims 1-44, wherein the cell is an immune effector cell.

48. The method of any one of claims 1-44 or 47, wherein the cell is a lymphocyte.

49. The method of any one of claims 1-44, 47, or 48, wherein the cell is a T-lymphocyte.

50. The method of any one of claims 1-44, 47, or 48, wherein the cell is a NK cell.

51. The method of any one of claims 1-44, wherein the cell is a stem cell.

52. The method of claim 51, wherein the stem cell is selected from the group consisting of an embryonic stem cell (ESC), an induced pluripotent stem cell (iPSC), a mesenchymal stem cell, or a tissue-specific stem cell.

53. A method comprising:
a) contacting a cell with (i) a first gRNA comprising a first targeting domain that binds to a first target sequence, and (ii) an RNA-guided nuclease that binds the first gRNA, thus forming a first ribonucleoprotein (RNP) complex under conditions suitable for the first gRNA of (i) to form and/or maintain the first RNP complex with the RNA-guided nuclease of (ii) and for the RNP complex to bind the first target sequence in the genome of the cell; and
b) contacting the cell with (iii) a second gRNA comprising a second targeting domain that binds to a second target sequence; and (iv) an RNA-guided nuclease that binds the second gRNA, thus forming a second ribonucleoprotein (RNP) complex under conditions suitable for the second gRNA of (iii) to form and/or maintain the second RNP complex with the RNA-guided nuclease of (iv) and for the second RNP complex to bind a second target sequence in the genome of the cell,
wherein steps (a) and (b) are performed sequentially in temporal proximity, separated by a time interval,
wherein the first targeting domain is different from the second targeting domain.

54. The method of claim 53, wherein the genetic modification of the first target sequence consists of an insertion or deletion at or immediately proximal to a site cut by the RNA-guided nuclease when bound to the first gRNA; and/or the genetic modification of the second target sequence consists of an insertion or deletion immediately proximal to a site cut by the RNA-guided nuclease when bound to the second gRNA.

55. The method of claim 53 or 54, wherein the method produces a subpopulation of translocation product cells, wherein each cell of the subpopulation comprises a translocation product comprising a portion of the genome comprising the first target sequence, a portion of the genome comprising the second target sequence, or both.

56. The method of claim 55, wherein the method produces fewer translocation product cells as compared to a method comprising contacting a cell with the second gRNA of (iii) prior to contacting the cell with the first gRNA of (i).

57. The method of claim 55 or 56, wherein the method produces at least 10% fewer translocation product cells as compared to a method comprising contacting the cell with the second gRNA of (iii) prior to contacting the cell with the first gRNA of (i).

58. The method of claim 55, wherein the method produces fewer translocation product cells as compared to a method comprising contacting the cell with the first gRNA of (i) and the second gRNA of (iii) at substantially the same time.

59. The method of claim 55 or 58, wherein the method produces at least 10% fewer translocation product cells as compared to a method comprising contacting the cell with the first gRNA of (i) and the second gRNA of (iii) at substantially the same time.

60. The method of any one of claims 53-59, wherein binding of the first RNP complex comprising (i) and (ii) to the first target sequence results in a genetic modification generated by a Non-Homologous End Joining (NHEJ) event.

61. The method of any one of claims 53-60, wherein binding of the RNP complex comprising (i) and (ii) to the first target sequence produces a fast-resolving double strand break.

62. The method of any one of claims 53-61, wherein binding of the second RNP complex comprising (iii) and (iv) to the second target sequence results in a genetic modification generated by a microhomology-mediated end joining (MMEJ) event.

63. The method of any one of claims 53-62, wherein binding of the second RNP complex comprising (iii) and (iv) to the second target sequence produces a slow-resolving double strand break.

64. The method of any one of claims 53-63, wherein the first target sequence is present in a first gene, a transcriptional control element operably linked thereto, or a portion of the gene and transcriptional control element.

65. The method of claim 64, wherein the genomic modification of the first target sequence results in reduced or eliminated expression of the product encoded by the first gene, or expression of a variant of the product expressed by wild-type cells of the same cell type that do not harbor a genomic modification in the first target sequence.

66. The method of claim 64 or 65, wherein the first gene encodes a first lineage-specific cell-surface antigen.

67. The method of claim 66, wherein the first lineage-specific cell-surface antigen is selected from the group consisting of CD33, CD19, CD123, CLL-1, CD30, CD5, CD6, CD7, CD38, and BCMA.

68. The method of any one of claims 53-67, wherein the second target sequence is present in a second gene, a transcriptional control element operably linked thereto, or a portion of the gene and transcriptional control element.

69. The method of claim 68, wherein the genomic modification of the second target sequence results in reduced or eliminated expression of the product encoded by the second gene, or expression of a variant of the product expressed by wild-type cells of the same cell type that do not harbor a genomic modification in the second target sequence.

70. The method of claim 68 or 69, wherein the second gene encodes a second lineage-specific cell-surface antigen.

71. The method of claim 70, wherein the second lineage-specific cell-surface antigen is selected from the group consisting of CD33, CD19, CD123, CLL-1, CD30, CD5, CD6, CD7, CD38, and BCMA.

72. The method of any one of claims 67-71, wherein the first lineage-specific cell-surface antigen is CD33.

73. The method of claim 71 or 72, wherein the second lineage-specific cell-surface antigen is CD19, CD5, or CLL-1.

74. The method of any one of claims 67-71, wherein the first lineage-specific cell-surface antigen is CD5.

75. The method of claim 74, wherein the second lineage-specific cell-surface antigen is CD33.

76. The method of claim 71 or 72, wherein the first lineage-specific cell-surface antigen is CD33 and the second lineage-specific cell-surface antigen is CLL-1.

77. The method of any one of claims 53-76, wherein the time interval between step (b) and step (c) is at least 4, 6, 8, 10, 12, 14, 16, 18, 20, 22, 24, 26, 28, 30, 32, 34, or 36 hours.

78. The method of any one of claims 53-77, wherein the RNA-guided nuclease of (ii) and/or the RNA-guided nuclease of (iv) is a CRISPR/Cas nuclease.

79. The method of claim 78, wherein the CRISPR/Cas nuclease is a Cas9 nuclease.

80. The method of claim 78, wherein the CRISPR/Cas nuclease is an spCas nuclease.

81. The method of claim 78, wherein the CRISPR/Cas nuclease is an saCas nuclease.

82. The method of claim 78, wherein the CRISPR/Cas nuclease is a Cpf1 nuclease.

83. The method of any one of claims 53-78, wherein the RNA-guided nuclease of (ii) is a Cas9 nuclease and the RNA-guided nuclease of (iv) is a Cpf1 nuclease.

84. The method of any one of claims 53-83, wherein the RNA-guided nuclease of (ii) is a Cpf1 nuclease and the RNA-guided nuclease of (iv) is a Cas9 nuclease.

85. The method of any one of claims 53-82, wherein the RNA-guided nuclease of (ii) and the RNA-guided nuclease of (iv) are Cpf1 nucleases.

86. The method of any one of claims 53-83, wherein the RNA-guided nuclease of (ii) and the RNA-guided nuclease of (iv) are Cas9 nucleases.

87. The method of any one of claims 53-86, wherein the contacting of (a) comprises introducing (i) and (ii) into the cell in the form of a pre-formed ribonucleoprotein (RNP) complex; and/or wherein the contacting of (b) comprises introducing (iii) and (iv) into the cell in the form of a pre-formed ribonucleoprotein (RNP) complex.

88. The method of claim 87, wherein the pre-formed ribonucleoprotein (RNP) complex is introduced into the cell via electroporation.

89. The method of any one of claims 53-88, wherein the contacting of (a) comprises introducing (i) and/or (ii) into the cell in the form of a nucleic acid encoding the gRNA of (i) and/or the RNA-guided nuclease of (ii); and/or
wherein the contacting of (b) comprises introducing (iii) and/or (iv) into the cell in the form of a nucleic acid encoding the gRNA of (i) and/or the RNA-guided nuclease of (ii).

90. The method of any one of claims 53-89, wherein the nucleic acid encoding the first gRNA of (i) and/or the RNA-guided nuclease of (ii) is an RNA, preferably an mRNA or an mRNA analog.

91. The method of any one of claims 53-90, wherein the nucleic acid encoding the second gRNA of (iii) and/or the RNA-guided nuclease of (iv) is an RNA, preferably an mRNA or an mRNA analog.

92. The method of any one of claims 53-91, wherein the first gRNA and/or the second gRNA comprises one or more nucleotide residues that are chemically modified.

93. The method of any one of claims 53-92, wherein the first and/or the second gRNA comprises one or more nucleotide residues that comprise a 2′O-methyl moiety.

94. The method of any one of claims 53-93, wherein the first and/or the second gRNA comprises one or more nucleotide residues that comprise a phosphorothioate.

95. The method of any one of claims 53-94, wherein the first and/or the second gRNA comprises one or more nucleotide residues that comprise a thioPACE moiety.

96. The method of any one of claims 53-95, wherein the cell is a hematopoietic cell.

97. The method of any one of claims 53-96, wherein the cell is a hematopoietic stem cell.

98. The method of any one of claims 53-96, wherein the cell is a hematopoietic progenitor cell.

99. The method of any one of claims 53-95, wherein the cell is an immune effector cell.

100. The method of any one of claims 53-95 or 99, wherein the cell is a lymphocyte.

101. The method of any one of claims 53-95, 99, or 100, wherein the cell is a T-lymphocyte.

102. The method of any one of claims 53-95, 99, or 100, wherein the cell is a NK cell.

103. The method of any one of claims 53-95, wherein the cell is a stem cell.

104. The method of claim 103, wherein the stem cell is selected from the group consisting of: an embryonic stem cell (ESC), an induced pluripotent stem cell (iPSC), a mesenchymal stem cell, or a tissue-specific stem cell.

105. A genetically engineered cell, or descendant thereof, produced by a method of any one of claims 1-104.

106. A cell population comprising a plurality of cells obtained by or obtainable by the method of any of any one of claims 1-104.

107. A pharmaceutical composition comprising the cell, or a descendant thereof, of claim 105 or the cell population of claim 106.

108. A method comprising administering to a subject in need thereof the cell, or descendant thereof, of claim 105, or cell population of claim 106, or the pharmaceutical composition of claim 107.

109. The method of claim 108, wherein the cell or descendant thereof or the cells of the cell population comprise a modification in a first gene relative to a wild-type counterpart cell and a modification to a second gene relative to a wild-type counterpart cell.

110. The method of claim 108 or 109, further comprising administering to the subject a therapeutically effective amount of at least one agent that targets a product encoded by the first gene or a wild-type copy thereof, wherein the agent comprises an antigen binding fragment that binds the product encoded by the first gene or a wild-type copy thereof.

111. The method of claim 110, wherein administration of the at least one agent targeting the product encoded by the first gene or a wildtype copy thereof occurs simultaneously or in temporal proximity with administration of the cell, or descendant thereof, of claim 105, the cell population of claim 106, or the pharmaceutical composition of claim 107.

112. The method of claim 110 or 111, wherein administration of the at least one agent targeting the product encoded by the first gene or a wildtype copy thereof occurs after administration of the cell, or descendant thereof, of claim 105, the cell population of claim 106, or the pharmaceutical composition of claim 107.

113. The method of claim 110 or 111, wherein administration of the at least one agent targeting the product encoded by the first gene or a wildtype copy thereof occurs before administration of the cell, or descendant thereof, of claim 105, the cell population of claim 106, or the pharmaceutical composition of claim 107.

114. The method of any one of claims 108-113, further comprising administering to the subject a therapeutically effective amount of at least one agent that targets a product encoded by the second gene or a wildtype copy thereof, wherein the agent comprises an antigen binding fragment that binds the product encoded by the second gene or a wildtype copy thereof.

115. The method of claim 114, wherein administration of the at least one agent targeting the product encoded by the second gene or a wildtype copy thereof occurs simultaneously or in temporal proximity with administration of the cell, or descendant thereof, of claim 105, the cell population of claim 106, or the pharmaceutical composition of claim 107.

116. The method of claim 114 or 115, wherein administration of the at least one agent targeting the product encoded by the second gene or a wildtype copy thereof occurs after administration of the cell, or descendant thereof, of claim 105, the cell population of claim 106, or the pharmaceutical composition of claim 107.

117. The method of any one of claims 114-116, wherein administration of the at least one agent targeting the product encoded by the second gene or a wildtype copy thereof occurs before administration of the cell, or descendant thereof, of claim 105, the cell population of claim 106, or the pharmaceutical composition of claim 107.

118. The method of any one of claims 114-117, wherein administration of the at least one agent targeting the product encoded by the second gene or a wildtype copy thereof occurs simultaneously or in temporal proximity with administration of the at least one agent targeting the product encoded by the first gene or a wildtype copy thereof.

119. The method of any one of claims 114-118, wherein administration of the at least one agent targeting the product encoded by the second gene or a wildtype copy thereof occurs after administration of the at least one agent targeting the product encoded by the first gene or a wildtype copy thereof.

120. The method of any one of claims 114-118, wherein administration of the at least one agent targeting the product encoded by the second gene or a wildtype copy thereof occurs before administration of the at least one agent targeting the product encoded by the first gene or a wildtype copy thereof.

121. The method of any one of claims 110-120, wherein the agent that targets a product encoded by the first gene or a wildtype copy thereof and/or the agent that targets a product encoded by the second gene or a wildtype copy thereof is cytotoxic agent.

122. The method of claim 121, wherein the cytotoxic agent is an antibody-drug conjugate or an immune effector cell expressing a chimeric antigen receptor (CAR).

123. The method of any one of claims 108-122, wherein the subject has a disease associated with cells expressing the modified gene or a wildtype copy thereof.

124. The method of any one of claims 108-123, wherein the subject has a cancer associated with cancer stem cells.

125. The method of any one of claims 108-124, wherein the subject has a hematopoietic malignancy.

126. The method of any one of claims 108-123, wherein the subject has an autoimmune disease.
